(19) United States
(12) Patent Application Publication
(10) Pub. No.: US 2017/0226598 A1
(43) Pub. Date: Aug. 10, 2017

(54) A BACILLUS METHYLOTROPHICUS STRAIN AND METHOD OF USING THE STRAIN TO INCREASE DROUGHT RESISTANCE IN A PLANT

(71) Applicant: THE ROYAL INSTITUTION FOR THE ADVANCEMENT OF LEARNING/MCGILL UNIVERSITY, MONTREAL (CA)

(72) Inventors: SUHA JABAJI, NOTRE-DAME-DE L'ILE PERROT (CA); FRANÇOIS GAGNÉ-BOURQUE, TROIS-RIVIÈRES (CA)

(21) Appl. No.: 15/328,274

(22) PCT Filed: Jul. 24, 2015
(86) PCT No.: PCT/CA2015/050699

§ 371 Date: Jan. 23, 2017

Related U.S. Application Data
(60) Provisional application No. 62/167,919, filed on May 29, 2015.
(60) Provisional application No. 62/130,263, filed on Mar. 9, 2015.
(60) Provisional application No. 62/028,578, filed on Jul. 24, 2014.

(51) Int. Cl. C12R 1/07; A01N 63/00; C12N 1/20

(52) U.S. Cl. CPC C12R 1/07; C12N 1/20; A01N 63/00

(57) ABSTRACT

A method of increasing drought resistance of a plant, the method comprising applying a Bacillus methylotrophicus or a composition thereof (i) to the plant or to a part of the plant; and/or (ii) to an area around the plant or plant part, in an amount effective to produce an increased drought resistance in the plant as compared to the drought stress resistance of the plant in the absence of said application of Bacillus methylotrophicus or composition, is described. A biologically pure culture of a 1-aminocyclopropane-1-carboxylate (ACC) deaminase deficient Bacillus methylotrophicus bacterium strain, or a mutant thereof able to induce drought resistance in a plant are also described.

### CROSS REFERENCE TO RELATED APPLICATIONS

This application is a PCT Application Serial No PCT/CA2015/* filed on Jul. 24, 2015 and published in English under PCT Article 21(2), which itself claims benefit of U.S. Provisional Application Ser. No. 62/028,578 filed on Jul. 24, 2014, U.S. Provisional Application Ser. No. 62/130,263 filed on Mar. 9, 2015, and U.S. Provisional Application Ser. No. 62/167,919 filed on May 29, 2015. All documents above are incorporated herein in their entirety by reference.

### STATEMENT REGARDING FEDERALLY SPONSORED RESEARCH OR DEVELOPMENT

N.A.

### FIELD

The disclosure relates to the use of a Bacillus methylotrophicus and a method for increasing drought resistance in a plant and to novel Bacillus methylotrophicus. In particular, embodiments of the present disclosure relate to the administration of Bacillus methylotrophicus to monocotyledonous plants to render them resistant to drought related stress. The resulting plants can be used in the production of human food crops, biofuels, biomass, and animal feed.

### REFERENCE TO SEQUENCE LISTING

Pursuant to 37 C.F.R. 1.821(c), a sequence listing is submitted herewith as an ASCII compliant text file named 11168_409_Seq_list_ST25.txt, that was created on Jul. 24, 2015 and having a size of ˜4.9 kilobytes. The content of the aforementioned file is hereby incorporated by reference in its entirety.

### BACKGROUND OF THE INVENTION

Plant growth-promoting bacteria (PGB) are mainly soil and rhizosphere-derived organisms that are able to colonize plant roots but with some having the ability of colonizing the internal tissues of plant organs. These are considered endophytes (Hardoim et al. 2008).

Irrespective of the mode of colonization, PGBs positively influence plant growth or reduce disease and abiotic stresses susceptibility through physical and chemical changes (Dimkpa, Weinand et al. 2009; Calvo, Nelson et al. 2014).

PGB mediated plant stress resistance have been reported in many studies and numerous genes induced by various stress conditions have been identified using molecular approaches (Timmusk and Wagner 1999; Zhang and Outlaw 2001; Sziderics, Rasche et al. 2007; Gagne-Bourque, Aliferis et al. 2013; Kasim, Osman et al. 2013; Gagné-Bourque, Mayer et al. 2015).

Rhizosphere microorganisms including PGBs are adapted to adverse conditions and may compensate for such detrimental conditions (Vivas, Marulanda et al. 2003; Marulanda, Barea et al. 2009; Marulanda, Azcón et al. 2010) and protect plants from the deleterious effects of drought thus increasing crop productivity under drought conditions. Endophytic bacteria may be even more important than rhizosphere bacteria, because they escape competition with rhizosphere microorganisms and achieve intimate contact with plant tissues. Several PGBs have been found to increase drought resistance in wheat, maize, lettuce, beans (Creus, Sueldo et al. 2004; Figueiredo, Burity et al. 2008; Marulanda, Barea et al. 2009; Vardharajula, Zulfikar Ali et al. 2011; El-Afry, El-Nady et al. 2012; Naveed, Mitter et al. 2014). A variety of mechanisms have been proposed behind microbial induced stress tolerance (IST) in plants (Yang, Kloepper et al. 2009). Some PGBs are known to promote root development thus improving the plant water absorption efficacy by extra production of the phytohormones, indole acetic acid (IAA), Gibberillic acid (GA), and cytokinins (Boiero, Perrig et al. 2007; Gagné-Bourque, Mayer et al. 2015).

Increase in total root system under stress conditions is the most commonly reported plant response mediated by PGB inoculation in various crops (Lucy, Reed et al. 2004; Wani and Khan 2010; Kasim, Osman et al. 2013). Investing more energy in developing a larger root system in order to optimize water extraction and minimizing water loss is a well-known drought avoidance mechanism by which plants manage to delay the consequence of drought (Chaves, Maroco et al. 2003; Meister, Rajani et al. 2014).

Others produce 1-aminocyclopropane-1-carboxylate (ACC) deaminase (Azevedo, Maccheroni Jr. et al.) that confers IST to drought stress in plants (Saleem, Arshad et al. 2007; Zahir, Munir et al. 2008) by reducing production of ethylene.

Studies on systemic tolerance to drought reported that inoculation with PGB enhanced drought tolerance via the increased transcription of drought-response genes (Sarma 2014), affecting the phytohormonal balance (Figueiredo et al. 2008) and sugar accumulation (Sandhya et al. 2010). Hence, some can induce modification in plant genes expression, increasing drought resistance associated gene like, ERD15 (Early Response to Dehydration 15) or DREB (Dehydration Responsive Element Protein) (Timmusk and Wagner 1999; Gagne-Bourque, Mayer et al. 2015).

PGB can induce metabolic adjustments leading to the modulation of several organic solutes like soluble sugars, starch and amino acids. More particularly, endophytes enhance drought and cold tolerance of tall fescue, maize and grapevine plants with higher and faster accumulation of stress-related metabolites (Vardharajula, Zulfikar Ali et al. 2011; Fernandez, Theocharis et al. 2012; Nagabhyru, Dinkins et al. 2013). Normally, soluble sugar content such as sucrose, glucose and fructose and raffinose, tends to be maintained or accumulated in the leaves of different droughted plants species (Spollen and Nelson 1994; Hare, Cress et al. 1998; Miazek, Bogdan et al. 2001; Taji, Ohsumi et al. 2002; Vardharajula, Zulfikar Ali et al. 2011; Bowne, Erwin et al. 2012). This is achieved at the expense of starch, which drastically declines (Chaves 1991). These sugars affect osmotic adjustment, and help in maintaining homeostasis allowing the plant to preserve its turgor pressure, thus normal function under water-limiting environment (Richardson, Chapman et al. 1992; Chaves, Maroco et al. 2003; Krasensky and Jonak 2012). In addition, these sugars help maintain the redox balance and act as reactive oxygen scavengers (Couée, Sulmon et al. 2006). Drought stress disrupts carbohydrate metabolism and sucrose level in leaves that spills over to decreased export rate, presumably due induced increased activity of acid invertase (Ruan, Jin et al. 2010). This may hamper the rate of sucrose export to the sink organs. During water stress, protein synthesis is slowed and hydrolysis may occur, promoting an increase in soluble nitrogen compounds such as amino acids (Farooq, Wahid et al. 2009; Krasensky and Jonak 2012). Levels of amino acids have been shown to increase in drought stressed plants (Bowne et al. 2012).

Several strains of Bacillus species, representing typical PGB colonize the rhizosphere and are reported to promote growth and enhance biotic and abiotic stress tolerance in a number of crops by exerting a number of characteristics enabling to mobilize soil nutrients and synthesize phytohormones without conferring pathogenicity ((Rodriguez and Fraga 1999; Saleem, Arshad et al. 2007; Van Loon 2007; Hardoim, van Overbeek et al. 2008; Ortiz-Castro, Valencia-Cantero et al. 2008; Niu, Liu et al. 2011; Wahyudi, Astuti et al. 2011; Truyens, Weyens et al. 2014; Lugtenberg and Kamilova 2009). The proposed mechanisms for plant growth promotion include increased nutrient availability, synthesizing plant hormones and production of volatiles (Ryu, Farag et al. 2003; Farag, Ryu et al. 2006). Considerable progress has been made in understanding the mechanisms underlying Bacillus-mediated tolerance to biotic stress, however, information on Bacillus strains mitigating abiotic stress symptoms is limited (Arkhipova, Prinsen et al. 2007; Ashraf and Foolad 2007; Vardharajula, Zulfikar Ali et al. 2011; Wolter and Schroeder 2012)) and the mechanisms underlying abiotic tolerance are largely elusive because most of the studies focus on evaluating plant growth promoting effects (Dimka et al. 2009).

Plants face various abiotic stresses among which drought is a major limiting factor both in growth and productivity of crops because it can elicit various biochemical and physiological reactions (Araus, Slaffer et al. 2002; Chaves, Maroco et al. 2003; Krasensky and Jonak 2012). Drought tolerance involves adaptation mechanism in which the plant produces osmolites and antioxidant molecules to help maintain cell turgor pressure, protect cellular macromolecules, membranes and enzyme from oxidative damage (Gill and Tuteja 2010; Krasensky and Jonak 2012). A correlation between drought tolerance and accumulation of compatible solutes such as carbohydrates, amino acids and ions to contribute to osmotic adjustments has been documented in grasses (Hanson and Smeekens 2009; Chen and Jiang 2010).

Adaptation to drought is an important acquirement of agriculturally relevant crops like food human crops and cool season grasses.

There is a need for alternative methods such as new PGBs conferring drought resistance to plants such as agriculturally relevant crops.

### SUMMARY OF THE INVENTION

The present invention provides the following items 1 to 27 and embodiments:

1. A method of increasing drought resistance of a plant, the method comprising applying a Bacillus methylotrophicus or a composition thereof (i) to the plant or to a part of the plant; and/or (ii) to an area around the plant or plant part, in an amount effective to produce an increased drought resistance in the plant as compared to the drought stress resistance of the plant in the absence of said application of Bacillus methylotrophicus or composition.

2. The method of item 1, wherein the Bacillus methylotrophicus exhibits one or more of (1) an ability to form sustaining endophytic populations in all tissues of the plant as well as in the rhizosphere; (2) an ability to avoid triggering the plant immune system; (3) an ability to reduce signs of wilting in the plant or increase survival time of the plant in drought conditions; (4) an ability to increase expression of at least one drought-responsive genes in the plant; (5) an ability to increase starch in the plant; (6) an ability to increase total soluble sugars in the plant; (7) an ability to increase DNA methylation in bacterized plant; (8) an ability to increase expression of at least one DNA methyltransferase in the plant; (9) an ability to maintain or increase crop biomass of the plant; (10) an ability to maintain or increase photosynthesis of the plant; (11) an ability to maintain or increase water conductance of the plant; (12) an ability to increase total amino acids content in roots and/or in shoots of the plant; (13) an ability to increase amino asparagine, glutamic acid and/or glutamine content in roots and/or in shoots of the plant; and (14) an ability to increase non-protein amino acid GABA in shoots and/or roots of the plant.

3. The method of item 1 or 2, wherein the Bacillus methylotrophicus exhibits one or more of (3) an ability to reduce signs of wilting in the plant or increase survival time of the plant in drought conditions; (4) an ability to increase expression of at least one drought-responsive genes in the plant; (5) an ability to increase starch in the plant; (6) an ability to increase total soluble sugars in the plant; (7) an ability to increase DNA methylation in bacterized plant; (8) an ability to increase expression of at least one DNA methyltransferase in the plant; (9) an ability to maintain or increase crop biomass of the plant; (10) an ability to maintain or increase photosynthesis of the plant; (11) an ability to maintain or increase water conductance of the plant; (12) an ability to increase total amino acids content in roots and/or in shoots of the plant; (13) an ability to increase amino asparagine, glutamic acid and/or glutamine content in roots and/or in shoots of the plant; and (14) an ability to increase non-protein amino acid GABA in shoots and/or roots of the plant, under drought conditions.

4. The method of any one of items 1 to 3, wherein the Bacillus methylotrophicus exhibits one or more of the characteristics (23) to (31) defined in Table 1.

5. The method of any one of items 1 to 4, wherein the Bacillus methylotrophicus is 1-aminocyclopropane-1-carboxylate (ACC) deaminase deficient.

6. The method of any one of items 1 to 5, wherein the plant is a poaceae plant.

7. The method of item 6, wherein the poaceae plant is a food crop plant.

8. The method of any one of items 1 to 7, wherein the amount effective is about 1×10⁸ CFU or more/plant, plant part, or area around a plant or plant part.

9. The method of any one of items 1 to 8, wherein the Bacillus methylotrophicus is in a seed of a second generation plant infected with the Bacillus methylotrophicus.

10. The method of any one of items 1 to 9, wherein the composition of Bacillus methylotrophicus comprises a polymer wherein said polymer is mixed and extruded with said Bacillus methylotrophicus in a proportion of 10 to 1.

11. The method of item 10, where the polymer is pea protein and/or alginate.

12. The method of any one of items 1 to 11, wherein the Bacillus methylotrophicus is of a strain comprising all of the biochemical characteristics of a Bacillus methylotrophicus deposited at the ATCC under accession no. * on Jul. 21, 2015, or a mutant thereof isolated from said strain and able to induce drought resistance to the plant.

13. A biologically pure culture of a 1-aminocyclopropane-1-carboxylate (ACC) deaminase deficient Bacillus methylotrophicus bacterium strain, or a mutant thereof able to induce drought resistance in a plant.

14. The Bacillus methylotrophicus bacterium strain, or mutant thereof of item 13, wherein the strain or mutant thereof exhibits one or more of (1) an ability to form sustaining endophytic populations in all tissues of the plant as well as in the rhizosphere; (2) an ability to avoid triggering the plant immune system; (3) an ability to reduce signs of wilting in the plant or increase survival time of the plant in drought conditions; (4) an ability to increase expression of at least one drought-responsive genes in the plant; (5) an ability to increase starch in the plant; (6) an ability to increase total soluble sugars in the plant; (7) an ability to increase DNA methylation in bacterized plant; (8) an ability to increase expression of at least one DNA methyltransferase in the plant; (9) an ability to maintain or increase crop biomass of the plant; (10) an ability to maintain or increase photosynthesis of the plant; (11) an ability to maintain or increase water conductance of the plant; (12) an ability to increase total amino acids content in roots and/or in shoots of the plant; (13) an ability to increase amino asparagine, glutamic acid and/or glutamine content in roots and/or in shoots of the plant; and (14) an ability to increase non-protein amino acid GABA in shoots and/or roots of the plant.

15. The Bacillus methylotrophicus bacterium strain, or mutant thereof of item 13 or 14, wherein the strain or mutant exhibits one or more of (3) an ability to reduce signs of wilting in the plant or increase survival time of the plant in drought conditions; (4) an ability to increase expression of at least one drought-responsive genes in the plant; (5) an ability to increase starch in the plant; (6) an ability to increase total soluble sugars in the plant; (7) an ability to increase DNA methylation in bacterized plant; (8) an ability to increase expression of at least one DNA methyltransferase in the plant; (9) an ability to maintain or increase crop biomass of the plant; (10) an ability to maintain or increase photosynthesis of the plant; (11) an ability to maintain or increase water conductance of the plant; (12) an ability to increase total amino acids content in roots and/or in shoots of the plant; (13) an ability to increase amino asparagine, glutamic acid and/or glutamine content in roots and/or in shoots of the plant; and (14) an ability to increase non-protein amino acid GABA in shoots and/or roots of the plant, under drought conditions.

16. The Bacillus methylotrophicus bacterium strain, or mutant thereof of any one of items 13 to 15, wherein the strain or mutant exhibits one or more of the characteristics (23) to (31) defined in Table 1.

17. A biologically pure culture of a bacterium strain comprising all of the biochemical characteristics of a Bacillus methylotrophicus deposited at the ATCC under accession no. * on Jul. 21, 2015, or a mutant thereof isolated from said strain and able to induce drought resistance to a plant.

18. A composition comprising a bacterium strain or mutant thereof as defined in any one of items 13 to 17, and at least one carrier.

19. The composition of item 18, wherein the carrier comprises a polymer wherein said polymer is mixed and extruded with said bacterium strain or mutant thereof in a proportion of about 10 to about 1.

20. The composition of item 19, where the polymer is pea protein and/or alginate.

21. A seed coated with a bacterium strain or mutant thereof as defined in any one of items 13 to 17, or with a composition as defined in any one of items 18 to 20.

22. A second or subsequent generation seed of a plant infected with bacterium strain or with a mutant thereof, the bacterium strain or a mutant thereof being as defined any one of items 13 to 17.

23. A method of increasing a plant's growth, the method comprising applying a bacterium strain or mutant thereof as defined in any one of items 13 to 17, or a composition as defined in any one of items 18 to 20, (i) to the plant or to a part of the plant; and/or (ii) to an area around the plant or plant part in an amount effective to produce an increased plant growth as compared to the growth of the plant in the absence of said application of Bacillus methylotrophicus or composition.

24. The method of item 23, wherein the plant is a poaceae plant.

25. The method of item 23, wherein the poaceae plant is a food crop plant.

26. The method of any one of items 23 to 25, wherein the amount effective is about 1×10⁸ CFU or more/plant, plant part, or area around a plant or plant part.

27. The method of any one of items 23 to 25, wherein the bacterium strain or mutant thereof is in a seed of a second generation plant infected with the bacterium strain or mutant thereof.

An embodiment of the present invention provides a method of increasing salt stress resistance of a plant, the method comprising applying a composition comprising Bacillus methylotrophicus B26 to the plant, to a part of the plant and/or to an area around the plant or plant part in an amount effective to produce an increased salt stress resistance in the plant or the part of the plant, wherein the salt stress resistance comprises greater drought tolerance.

Another embodiment provides a method of increasing water stress resistance of a plant, the method comprising applying a composition comprising Bacillus methylotrophicus B26 to the plant, to a part of the plant and/or to an area around the plant or plant part in an amount effective to produce an increased water stress resistance in the plant, the part of the plant, wherein the water stress resistance leads to greater drought tolerance.

Still another embodiment provides a method of increasing water stress resistance of a plant, the method comprising applying a composition comprising Bacillus methylotrophicus B26 to the plant, to a part of the plant and/or to an area around the plant or plant part in an amount effective to produce an increased water stress resistance in the plant or the part of the plant, wherein the water stress resistance leads to greater drought tolerance, wherein the plant is selected from the group consisting of monocot plants.

Another embodiment provides a method of increasing water stress resistance of a plant, the method comprising applying a composition comprising Bacillus methylotrophicus B26 to the plant, to a part of the plant and/or to an area around the plant or plant part in an amount effective to produce an increased water stress resistance in the plant or the part of the plant, wherein the water stress resistance leads to greater drought tolerance, wherein the monocot plant is a biomass crop plant.

Another embodiment provides a method of increasing water stress resistance of a plant, the method comprising applying a composition comprising Bacillus methylotrophicus B26 to the plant, to a part of the plant and/or to an area around the plant or plant part in an amount effective to produce an increased water stress resistance in the plant or the part of the plant, wherein the water stress resistance leads to greater drought tolerance, wherein the monocot plant or the biomass crop plant is selected from the group consisting of switchgrass (Panicum virgatum), giant reed (Arundo donax), reed canarygrass (Phalaris arundinacea), Miscanthusxgiganteus, Miscanthus sp., Sericea lespedeza (Lespedeza cuneata), ryegrass (Lolium multiflorum, Lolium sp.), timothy (Phleum pretense), kochia (Kochia scoparia), turf grass, sunn hemp, kenaf, bahiagrass, bermudagrass, dallisgrass, pangolagrass, big bluestem, indiangrass, fescue (Festuca sp.) including tall fescue, Dactylis sp., Brachypodium distachyon, smooth bromegrass, orchardgrass and kentucky bluegrass.

Another embodiment provides a method of increasing growth of a plant, the method comprising applying a composition comprising Bacillus methylotrophicus B26 to the plant, to a part of the plant and/or to an area around the plant or plant part in an amount effective to produce an increased growth in the plant or the part of the plant, wherein the growth promoting effect leads to greater drymass, wherein the monocot plant or the biomass crop plant is selected from the group consisting of switchgrass (Panicum virgatum), giant reed (Arundo donax), reed canarygrass (Phalaris arundinacea), Miscanthus×giganteus, Miscanthus sp., Sericea lespedeza (Lespedeza cuneata), ryegrass (Lolium multiflorum, Lolium sp.), timothy (Phleum pretense), kochia (Kochia scoparia), turf grass, sunn hemp, kenaf, bahiagrass, bermudagrass, dallisgrass, pangolagrass, big bluestem, indiangrass, fescue (Festuca sp.) including tall fescue, Dactylis sp., Brachypodium distachyon, smooth bromegrass, orchardgrass or kentucky bluegrass.

Another embodiment provides a method of increasing water stress resistance of a plant, the method comprising applying a composition comprising Bacillus methylotrophicus B26 to the plant, to a part of the plant and/or to an area around the plant or plant part in an amount effective to produce an increased water stress resistance in the plant or the part of the plant, wherein the water stress resistance leads to greater drought tolerance, wherein the monocot plant or the biomass crop plant is selected from the group consisting of corn, rice, triticale, wheat, barley, oats, rye grass and millet.

Another embodiment provides a method of increasing growth of a plant, the method comprising applying a composition comprising Bacillus methylotrophicus B26 to the plant, to a part of the plant and/or to an area around the plant or plant part in an amount effective to produce an increased growth in the plant or the part of the plant, wherein the growth promoting effect leads to greater dry mass, wherein the monocot plant or the biomass crop plant is selected from the group consisting of corn, rice, triticale, wheat, barley, oats, rye grass and millet.

A further embodiment provides a method of increasing water stress resistance of a plant, the method comprising applying a composition comprising Bacillus methylotrophicus B26 to the plant, to a part of the plant and/or to an area around the plant or plant part in an amount effective to produce an increased water stress resistance in the plant or the part of the plant, wherein the water stress resistance leads to greater drought tolerance comprising administering the Bacillus methylotrophicus B26 composition in an amount effective to produce a drought resistant bacterized biomass crop plant prolonging its resistance to water from about * days to about * days compared to an non-bacterized biomass crop plant.

A further embodiment provides a method of increasing water stress resistance of a plant, the method comprising applying a composition comprising Bacillus methylotrophicus B26 to the plant, to a part of the plant and/or to an area around the plant or plant part in an amount effective to produce an increased salt stress resistance in the plant or the part of the plant, wherein the water stress resistance leads to greater drought tolerance comprising administering the Bacillus methylotrophicus B26 composition to the plant, to a part of the plant and/or to an area around the plant or plant part in an effective amount up to about 1×10⁸ CFU/plant, plant part, or area around a plant or plant part.

A further embodiment provides a method of increasing water stress resistance of a plant, the method comprising applying a composition comprising Bacillus methylotrophicus B26 to the plant, to a part of the plant and/or to an area around the plant or plant part in an amount effective to produce an increased water stress resistance in the plant or the part of the plant, wherein the water stress resistance leads to greater drought tolerance and wherein the composition comprises a seed of a second generation plant infected with the endophyte Bacillus methylotrophicus B26.

A further embodiment provides for a method of increasing water stress resistance to a plant, the method comprising applying a composition comprising Bacillus methylotrophicus and a material that forms a microsphere incorporating said Bacillus and wherein said material consists of a polymer that can be mixed with the bacteria at a proportion of 10:1 and both can be extruded as microspheres.

Another embodiment provides for a method of increasing water stress resistance to a plant, the method comprising applying microspheres consisting of bacteria and a polymer, wherein the polymer is selected from the group of alginate and pea protein.

Another embodiments provides for a method of increasing water stress resistance to a plant, the method comprising applying microspheres consisting of bacteria and a polymer, wherein the microspheres can be freeze dried after which said microspheres can be stored at either −15 C, 4 C or 22 C.

A further embodiment provides for a method of increasing water stress resistance to a plant wherein microspheres containing Bacillus methylotrophicus B26 are applied at the time of planting or seeding and where a continuously high level of Bacillus subtilis B26 in the soil can be achieved by reapplication on already planted plants.

According to another aspect of the present invention, there is provided a method for increasing the ability of a bacterial strain to induce drought resistance in a plant comprising interspecific (i.e. between the bacterial species of the present invention and another bacterial species of the Firmicutes phylum. In a more specific embodiment, the Firmicutes phylum bacterium is a Bacilli. In another embodiment, the Bacilli bacterium is a Bacillales. In a more specific embodiment, the Bacillales is a Bacillaceae. In a more specific embodiment, the Bacillaceae bacterium is a Bacillus spp.) or intraspecific protoplasm fusion of the bacterial strain with a bacterial strain of the present invention (e.g., a Bacillus methylotrophicus strain as defined herein such as B26 or a mutant thereof as defined herein able to induce drought resistance in a plant). In a more specific embodiment, the protoplasm fusion is intraspecific (between the bacterium of the present invention and another Bacillus methylotrophicus). Drought resistance traits can be conferred from one species to another by protoplast fusion (Hennig et al. 2015). Protoplasm fusion has been used between to transfer traits between bacteria. (Ran et al. 2013; Agbessi et al. 2003).

According to another aspect of the present invention, there is provided a method for increasing the ability of a bacterial strain to increase a plant's growth comprising interspecific as defined above or intraspecific protoplasm fusion of the bacterial strain with a bacterial strain of the present invention (e.g., a Bacillus methylotrophicus strain as defined herein such as B26 or a mutant thereof as defined herein able to induce drought resistance in a plant). In a more specific embodiment, the protoplasm fusion is intraspecific (between the bacterium of the present invention and another Bacillus methylotrophicus.

Other objects, advantages and features of the present invention will become more apparent upon reading of the following non-restrictive description of specific embodiments thereof, given by way of example only with reference to the accompanying drawings.

### BRIEF DESCRIPTION OF THE FIGURES

FIG. 1 shows that the inoculation of Bacillus methylotrophicus strain B26 improved production of biomass and seeds in Brachypodium distachyon plants. Plants were visually compared as bacterized plants and non-bacterized plants. Initial tests involved culture dependent tests, such as the determination of colony forming units of endophytes, culture independent methods, such as quantitative PCR, and agronomic measurements, such as biomass.

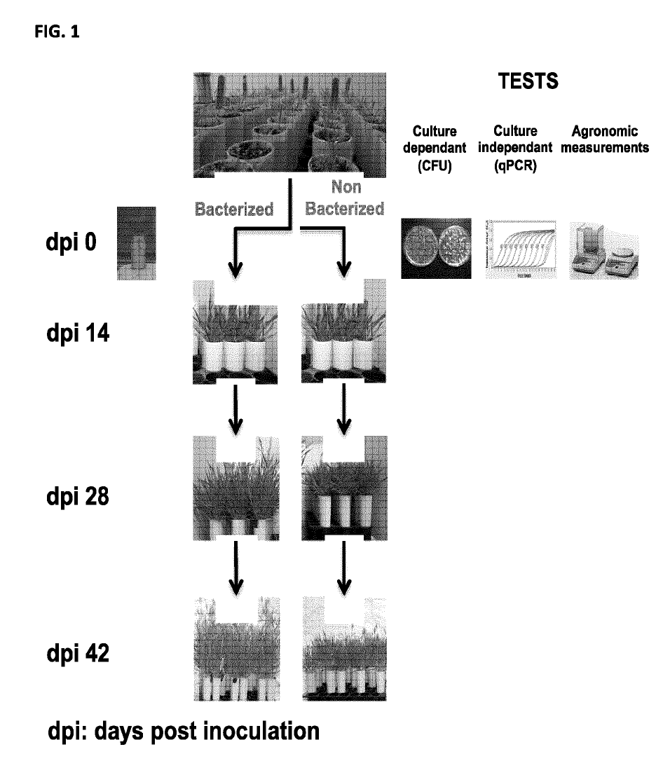

FIG. 2 shows the comparison in plant growth between bacterized and non-bacterized Brachypodium distachyon plants in terms of total plant height (A), shoot dry biomass (B), root drymass (C), number of leaves (D), and number of seeds (E). (F) presents a photographic comparison of bacterized and non-bacterized Brachypodium distachyon whole plants.

FIG. 3 shows the comparison of number of seed heads (A) and number of spikelets (B) generated in Brachypodium non-bacterized (non-inoculated) and bacterized (inoculated) with B. methylotrophicus strain B26.

FIG. 4 shows the detection of B. methylotrophicus B26 by PCR in different tissues using species-specific primers. Lane 1: pure B. methylotrophicus B26; Lane 2: no template control; Lanes 3 to 5: non-bacterized plant tissues at D63 of root, shoot and seed, respectively; Lanes 6 to 8: bacterized plant tissues at D63 of root, shoot, seed, respectively; and Lanes 9 to 10: plant tissue of second generation bacterized plants at D28 of root and shoot, respectively.

FIG. 5 shows the relative transcript accumulation of PR1-like gene, a marker of immune response, in bacterized (inoculated) and non-bacterized (non-inoculated) plants from 0 to 168 hours post inoculation with B. methylotrophicus strain B26 (A) or Brachypodium distachyon Bd21 plants treated or not with Salicylic Acid (SA) (B).

FIG. 6 shows the methodology used to subject Brachypodium distachyon to chronic water stress for results presented herein.

FIG. 7 shows the relative transcript accumulation of drought-responsive genes. The relative mRNA abundance of DREB2B-like (A, B), DHN3-like (C, D) and LEA-14-A-like (E, F) in non-bacterized (non-inoculated) and bacterized (inoculated) Brachypodium plants before and 90 mins after uprooting (A, C, E) (acute drought stress) or before and after five and eight days of chronic drought stress (B, D, F) are depicted. * represent a statistically significant difference.

FIG. 8 shows transmission electron microscopy (TEM) micrographs of colonized Brachypodium tissues with B. methylotrophicus B26. (A). Cross section of root xylem with numerous bacterial cells present inside the vessel elements (arrows). (B, C). Leaf mesophyll cells and bundle sheath (inset) with bacterial cells (arrows). (D). Vessel elements of xylem stem tissue showing B26 in and outside the vessel elements. (E). Cross section of seed with B26 cells. (F). Cross section of chloroplast of a leaf bundle sheath cell from a colonized leaf. Notice the abundance of starch granules (“S” in panel) and the integrity of the thylakoids. (G). B. methylotrophicus B26 cells grown in pure culture.

FIG. 9 shows effects of drought stress on non-bacterized and bacterized Brachypodium plants. Non-bacterized (left) and bacterized (right) Brachypodium plants (A) before or (B and C) after one and two hours of acute drought stress. Pictures of non-bacterized (left) and bacterized (right) Brachypodium plants were also taken at (E) 0 day, (F) 5 days and (G) 8 days after last watering.

FIG. 10 shows soluble sugars and starch concentrations of bacterized (inoculated) and non-bacterized (non-inoculated) plants under control and drought conditions. (A) 5 days and (B) 8 days post watering * Represent a statistically significant difference.

FIG. 11 shows global DNA methylation variations in bacterized (inoculated) and non-bacterized (non-inoculated) Brachypodium plants under control and drought conditions. (A) Before and after one hour (1H) of acute drought stress. (B) Before and after five (D5) and eight (D8) days of chronic drought stress. * Represent a statistically significant difference.

FIG. 12 shows relative transcript accumulation of DNA methyltransferases in bacterized (inoculated) and non-bacterized (non-inoculated) Brachypodium plants under control and drought conditions. Relative mRNA abundance of methyltransferases 1-like (MET1B-like) (A, B), chromomethylase 3-like (CMT3-like) (C, D) and domains-rearranged methyltransferases 2-like (DRM2-like) (E, F) before and 90 mins after (from left to right respectively) uprooting non-bacterized (non-inoculated) and bacterized (inoculated) plants (A, C, E) or before and after five and eight days (from left to right respectively) after last watering of non-bacterized (non-inoculated) and bacterized (inoculated) plants (B, D, F). * Represent a statistically significant difference.

FIG. 13 shows the increase in plant growth of bacterized (inoculated) plants compared to non-bacterized (non-inoculated) plants, namely wheat (A), barley (B), and oat (C). Panel D summarizes the respective dry biomass of A, B, and C.

FIG. 14 shows the increase in plant growth of bacterized (inoculated) plants compared of non-bacterized (non-inoculated) plants, namely reed Canary grass (A), Smooth Bromegrass (B) and Timothy (C). Panel D summarizes the respective dry biomass of A, B, C.

FIG. 15 shows a formulation of Bacillus methylotrophicus B26 in microbeads, i.e. pea protein isolate-alginate microspheres prepared via extrusion of a suspension comprising a bacteria to polymer ratio of 1:10 (v/v) (A). Panels B1 to B3 represent a Scanning Electron Microscopy (SEM) image at different levels of magnification. B-1 shows the outside surface of a microbead, B-2 shows the incorporation of Bacillus methylotrophicus B26 spores (arrows), and B-3 shows the inside of a microsphere including Bacillus methylotrophicus B26 spores (arrows). Panel C shows microbeads used for the inoculation of plants as further described in FIG. 17.

FIG. 16 shows the survival rates of free Bacillus methylotrophicus B26 cells (A) and of encapsulated B. methylotrophicus B26 cells (B) under different temperature conditions. * represents a statistically significant difference.

FIG. 17 shows the effect of Bacillus methylotrophicus B26 loaded microspheres on Brachypodium and timothy plants with a pre-inoculation or pre-planting treatment and with a post-inoculation or post-planting treatment including non-inoculated controls. Panel A provides a visual comparison of the bacterized (inoculated) and non-bacterized (non-inoculated) Brachypodium plants obtained with the pre-inoculation or pre-planting treatment and with a post-inoculation or post-planting treatment. Panel B shows the concentration of Bacillus methylotrophicus B26 in top soil over the period of 56 days when Bacillus methylotrophicus B26 loaded microspheres are applied to topsoil at the time of seeding Brachypodium or timothy, i.e. according to the pre-inoculation or pre-planting treatment mode. Panel C shows the concentration of Bacillus methylotrophicus B26 in top soil over the period of 35 days when Bacillus methylotrophicus B26 loaded microspheres are applied to topsoil when Brachypodium or timothy plants have reached an age of 21 days according to the post-inoculation or post-planting treatment mode.

FIG. 18 shows a flow chart of the experimental set-up of non-inoculated (NI) and inoculated (I) timothy grass with Bacillus methylotrophicus B26 grown under well-watered (WW) and stress conditions (DRY). WSP=Weeks post seeding and W=Weeks. H=Harvest date.

FIG. 19 summarizes dry mass of shoot and root (A, B), photosynthesis (C, D) and water conductance (E, F) of timothy grass inoculated (endophyte) or not (non-endophyte) with B. methylotrophicus B26 after 4 (harvest 1) (A, C, E) and 8 (harvest 2) (B, D, F) weeks of withholding water. *=Represents a statistically significant difference. All statistical analyses were performed by one-way ANOVA. The significance of the effect of the treatments was determined via Tukey HSD with a magnitude of the F-value (P=0.05). Harvest 1 (4 weeks of withholding water) and Harvest 2 (8 weeks of withholding water) β were analyzed separately.

FIG. 20 shows the dynamics of B. methylotrophicus B26 in soil and in timothy grass under well-watered (WW) and stress conditions (DRY). (A) Colony forming units (CFU) number estimated in rhizosphere soil, shoot and root tissues after 4 weeks (Harvest 1) and 8 weeks (Harvest 2) of withholding water. (B) Copy number of B. methylotrophicus B26 in shoot and root tissues of timothy exposed to 4 weeks (Harvest 1) and 8 weeks of stress (Harvest 2). (C) Copy number of DNA of strain B26 estimated in fresh weight in different tissues using species-specific primers. Lane +, B. methylotrophicus B26 pure DNA; Lane −, no template; Lanes 1, 3, 5, 7, 9, 11, 13 and 15 represent inoculated plant tissues of root and shoot. Lanes 2, 4, 6, 8, 10, 12, 14 and 16 represent non-inoculated plant tissues of root and shoot.

FIG. 21 depicts a multivariate analysis of Harvest 1 (A) and Harvest 2 (B). Projections to latent structures-discriminant analysis (OPLS-DA) score plot. The ellipse represents the Hotelling T² with 95% confidence interval. Four biological replications each consisting of ten plants were performed per treatment (Q²(cum); cumulative fraction of the total variation of the X's that can be predicted y the extracted components, R²X and R²Y; the fraction of the sum of squares of all X's and Y's explained by the current component, respectively).

FIG. 22 shows a discriminant analysis (OPLS-DA) coefficient plot for selected influential factors for the observed separation between the inoculated and non-inoculated under water stressed conditions (DRY) (A) and well-watered (WW) (B) after 4 weeks of withholding water (Harvest 1) displayed with a jack-knifed confidence intervals (P=0.05). List of abbreviation: Ala=alanine, Arg=arginine, Asn=asparagine, Asp=aspartic acid, Gln=glutamine, Glu=glutamic acid, Gly=glycine, His=histidin, Ile=isoleucine, Leu=leucine, Lys=leucine, Lys=lysine, Met=methionine, Phe=phenylalanine, Pro=proline, Ser=serine, Thr=threonine, Tyr=tyrosine, Val=valine, Orn=Ornithine, AA_TOT=Total amino acid, SSTot=Total soluble sugars, CHOTOT=total carbohydrate, AABA=α-aminobutyric acid, HPM=fructan, GABA=γ-aminobutyric acid, items labeled with R refer to their presence in Roots, Items labelled with L refer to their presence in Leaves and shoots. Metabolites increased in the inoculated plant appear on the left side of each panel.

FIG. 23 shows a discriminant analysis (OPLS-DA) coefficient plot for selected influential factors for the observed separation between the inoculated and non-inoculated under water stressed conditions (DRY) (A) and well-watered (WW) (B) after 8 weeks of withholding water (Harvest 2) displayed with a jack-knifed confidence intervals (P=0.05). List of abbreviation: Ala=alanine, Arg=arginine, Asn=asparagine, Asp=aspartic acid, Gln=glutamine, Glu=glutamic acid, Gly=glycine, His=histidin, Ile=isoleucine, Leu=leucine, Lys=leucine, Lys=lysine, Met=methionine, Phe=phenylalanine, Pro=proline, Ser=serine, Thr=threonine, Tyr=tyrosine, Val=valine, Orn=Ornithine, AATOT=Total amino acid, SSTOT=Total soluble sugars, CHOTOT=total carbohydrate, AABA=α-aminobutyric acid, HPM=fructan, GABA=γ-aminobutyric acid, items labeled with R refer to their presence in Roots, Items labelled with L refer to their presence in Leaves and shoots. Metabolites increased in the inoculated plant appear on the left side of each panel.

FIG. 24 depicts a metabolic pathway map of inoculated timothy plants after 4 and 8 weeks of withholding water. Fluctuation in the inoculated timothy metabolic pathway leading to amino acid production of shoot (A) and root (B) tissues of bacterized timothy at harvest 1 and 2. Variable relative concentrations are coded using a color based on the means of scaled and centered OPLS regression coefficients (CoeffCS) from 4 biological replications. Dashed lines symbolize a multistep and solid lines one-step reactions. List of abbreviation: AATOT=Total amino acid, SSTOT=Total soluble sugars, CHOTOT=total carbohydrate AABA=α-aminobutyric acid, GABA=γ-aminobutyric acid.

FIG. 25 summarizes soil moisture (A) and water potential (kPa) (B) of bacterized (Inoculated) or not (Non-inoculated) timothy plants with strain B26 and exposed or not to water stress after 4 (H1) and 8 weeks (H2).

FIG. 26 shows a principal component analysis PC1/PC2 score plots of (A) Inoculated and non-inoculated. (B) Well-watered (WW) and dry (DRY) treatment and (C) harvest 1 (H1) and harvest 2 (H2).

FIG. 27 shows the lack of ACC deaminase gene by PCR analysis in B. methylotrophicus B26 using ACC1 and ACC2 primer sets. Panel A shows: Lane + B. methylotrophicus B26 DNA using B26 specific primer set; Λ 100 bp DNA ladder from FroggaBio; Lane − No template DNA on B26 specific primer set; Lane 1 B. methylotrophicus B26 DNA using ACC1 primer set; Lane 2 No template DNA using ACC1 primer set; Lane 3 B. methylotrophicus B26 DNA using ACC2 primer set; Lane 4 No template DNA using ACC2 primer set. Panel B shows a PCR analysis using the following primers: Lane + B. methylotrophicus B26 DNA using B26 specific primer set; Lane − No template DNA on B26 specific primer set; Lane 1 B. methylotrophicus B26 DNA using ACC3 primer set; Lane 2 No template DNA using ACC3 primer set; Lane 3 B. methylotrophicus B26 DNA using ACC_general primer set; and Lane 4 No template DNA using ACC_general primer set.

FIG. 28 shows the lack of growth of B. methylotrophicus in broth and on agar plates with ACC as single nitrogen source.

### DETAILED DESCRIPTION OF THE INVENTION

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

In the present description, a number of terms are extensively utilized. In order to provide a clear and consistent understanding of the specification and claims, including the scope to be given such terms, the following definitions are provided.

The use of the word “a” or “an” when used in conjunction with the term “comprising” in the claims and/or the specification may mean “one” but it is also consistent with the meaning of “one or more”, “at least one”, and “one or more than one”. Throughout this specification, unless the context requires otherwise, the words “comprise,” “comprises” and “comprising” will be understood to imply the inclusion of a stated step or element or group of steps or elements but not the exclusion of any other step or element or group of steps or elements.

Throughout this application, the term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

As used herein, the term “consists of” or “consisting of” means including only the elements, steps, or ingredients specifically recited in the particular claimed embodiment or claim.

Terms and symbols of genetics, molecular biology, biochemistry and nucleic acid used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like. All terms are to be understood with their typical meanings established in the relevant art.

The present invention concerns nonpathogenic Bacillus methylotrophicus (illustrated by a Bacillus, which is now identified as a Bacillus methylotrophicus strain B26 submitted at the ATCC under accession number * filed Jul. 21, 2015) and mutants thereof displaying drought resistance, and, in more specific embodiments, plant growth enhancing activities.

A mutant of the B26 strain deposited at the ATCC under access no * may or may not have the same identifying biological characteristics of the B26 strain, as long as it can induce drought resistance in plants that it colonizes. Illustrative examples of suitable methods for preparing mutants of the microorganism of the present invention (i.e. Bacillus methylotrophicus) include, but are not limited to: interspecific or intraspecific protoplast fusion according to the CRISPR-Cas9 method (Ran et al. 2013); mutagenesis by irradiation with ultraviolet light or X-rays; or by treatment with a chemical mutagen such as nitrosoguanidine (N-methyl-N′-nitro-N-nitrosoguanidine), methylmethane sulfonate, nitrogen mustard and the like; gene integration techniques, such as those mediated by insertional elements or transposons or by homologous recombination of transforming linear or circular DNA molecules; and transduction mediated by bacteriophages such as P1. These methods are well known in the art and are described, for example, in J. H. Miller, Experiments in Molecular Genetics, Cold Spring Harbor Laboratory Press, Cold Spring Harbor, N.Y. (1972); J. H. Miller, A Short Course in Bacterial Genetics, Cold Spring Harbor Laboratory Press, Cold Spring Harbor, N.Y. (1992); M. Singer and P. Berg, Genes & Genomes, University Science Books, Mill Valley, C A (1991); J. Sambrook, E. F. Fritsch and T. Maniatis, Molecular Cloning: A Laboratory Manual, 2d ed., Cold Spring Harbor Laboratory Press, Cold Spring Harbor, N.Y. (1989); P. B. Kaufman et al., Handbook of Molecular and Cellular Methods in Biology and Medicine, CRC Press, Boca Raton, Fla. (1995); Methods in Plant Molecular Biology and Biotechnology, B. R. Glick and J. E. Thompson, eds., CRC Press, Boca Raton, Fla. (1993); and P. F. Smith-Keary, Molecular Genetics of Escherichia coli, The Guilford Press, New York, N.Y. (1989).

Mutant strains derived from the B26 strain using known methods are then preferably selected or screened for ability to induce drought resistance to plants.

The current screening assay for drought resistance inducing bacteria involves determining the bacteria's ACC deaminase activity, as the latter is generally considered essential for drought resistance. The Bacillus methylotrophicus of the present invention are however ACC deaminase deficient. Mutants can be selected by methods described in Examples herein.

Additional useful Bacillus methylotrophicus of the present invention may be identified or defined as exhibiting one or more one or more (two or more; three or more; four or more; five or more, six of more, seven or more, eight or more, nine or more, ten or more, eleven or more, twelve or more, thirteen or more or fourteen) of the following characteristics: (1) ability to form sustaining endophytic populations in all bacterized plant tissues as well as in the rhizosphere (e.g., following methods as described in Examples 1, 3, 16 and 18); (2) ability to avoid triggering the plant immune system (e.g., following methods as described in Examples 1 and 4); (3) ability to reduce signs of wilting of a bacterized plant or increase survival time of the plant in drought conditions (e.g., following methods as described in Examples 5, 6, 16 and 17); (4) increase expression of at least one (at least two or at least three) drought-responsive genes such DREB2B, LEA-14, and DHN3 in a bacterized plant subjected to drought conditions or in well-watered conditions (e.g., following methods as described in Examples 5 and 7); (5) ability to increase starch in bacterized plant subjected to drought conditions or well-watered conditions (e.g., following methods as described in Examples 5, 9 and 22-24); (6) ability to increase total soluble sugars in bacterized plant subjected to drought conditions or well-watered conditions (e.g., following methods as described in Examples 5, 9, 16 and 20); (7) ability to increase DNA methylation in bacterized plant subjected to drought conditions or well-watered conditions (e.g., following methods as described in Examples 5 and 10); (8) ability to increase expression of at least one (or at two or at least three) DNA methyltransferase(s) (e.g., MET1, CMT3 and DRM2) in bacterized plants subjected to drought conditions or well-watered conditions (e.g., following methods as described in Examples 5 and 11); (9) ability to maintain or increase crop biomass of bacterized plants subjected to drought conditions or well-watered conditions (e.g., following methods as described in Examples 1-2, 12-13 and 16-17); (10) ability to maintain or increase photosynthesis of bacterized plants subjected to drought conditions or well-watered conditions (e.g., following methods as described in Examples 16-17); (11) ability to maintain or increase water conductance of bacterized plants subjected to drought conditions or well-watered conditions (e.g., following methods as described in Examples 16-17); (12) ability to increase total amino acids content in roots and/or in shoots of bacterized plants subjected to drought conditions or well-watered conditions (e.g., following methods as described in Examples 16 and 21-28); (13) ability to increase specific amino acids content (e.g. asparagine, the precursors of proline, glutamic acid and/or glutamine) in roots and/or in shoots of bacterized plants subjected to drought conditions or well-watered conditions (e.g., following methods as described in Examples 16 and 21-28); and (14) ability to increase non-protein amino acid GABA in shoots exposed to stress and roots of stressed and not stressed plants (e.g., following methods as described in Examples 16 and 21-28). The increased in characteristics (3) to (14) are as compared to the corresponding characteristic(s) in a non-bacterized plant (i.e. not bacterized with a bacterium of the present invention). In specific embodiments, the Bacillus methylotrophicus is ACC deaminase deficient.

Additional useful Bacillus methylotrophicus of the present invention may further be identified or defined as exhibiting one or more (two or more; three or more; four or more; five or more, six of more, seven or more, or eight) of the following additional characteristics: (15) ability to increase height of bacterized plant (e.g., following methods as described in Examples 1 and 2); (16) ability to increase root and/or shoot dry weight of bacterized plant (e.g., following methods as described in Examples 1 and 2); (17) ability to increase number of seeds of bacterized plant (e.g., following methods as described in Examples 1 and 2); (18) ability to increase number of spikelets of bacterized plant (e.g., following methods as described in Examples 1 and 2); (19) ability to increase number of leaves of bacterized plant; (20) ability to increase total tiller number of bacterized plant; (21) ability to increase ratio of reproductive tiller/total tiller; and (22) ability to increase chlorophyll content leading to darker leaves of bacterized plant. The increase in characteristics (15) to (22) are as compared to the corresponding characteristic(s) in a non-bacterized plant (i.e. not bacterized with a bacterium of the present invention).

Additional useful Bacillus methylotrophicus of the present invention may be identified or defined as a bacterium resulting from the intraspecific protoplasm fusion of the Bacillus methylotrophicus B26 or a mutant thereof isolated from said strain and able to induce drought resistance to a plant, with another Bacillus methylotrophicus.

Additional useful Bacillus methylotrophicus of the present invention may further be identified or defined as exhibiting one or more of the following additional characteristics, namely the ability to express one or more (two or more; three or more; four or more; five or more, six of more or seven) of the following metabolites:

TABLE 1
Metabolites identified in the supernatant of Bacillus methylotrophicus B26.

| | Chemical | Monisotopic | KEGG | KEGG | |
|---|---|---|---|---|---|
| Compound | formula | Mass | ID | pathways | Structure |
| (23) Indole-3- acetate | C₁₀H₉NO₂ | 175_0633 | C00954 | ko00380 Tryptophan metabolism, ko04075 Plant hormone signal transduction | 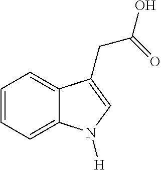 [SMILES: O=C(O)Cc1c[nH]c2ccccc12] |
| (24) Methyl- indole-3- acetate | C₄₅H₆₈N₁₀O₁₅ | 189_079 | NA | NA | 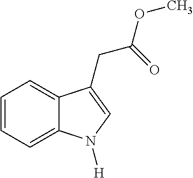 |
| (25) Bacillomycin- D (iturin) | C₄₅H₆₈N₁₀O₁₅ | 988_4866 | C12267 | ko01054 Nonribosomal peptide structures | 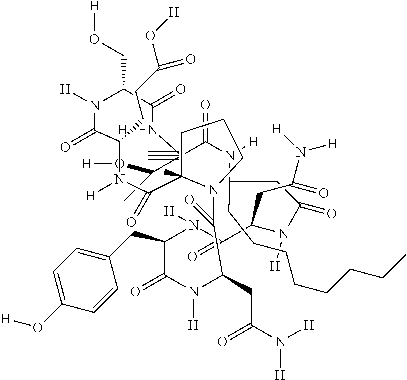 |
| (26) Iturin D | C₄₈H₇₄N₁₂O₁₄ | 1042_5447 | NA | NA | 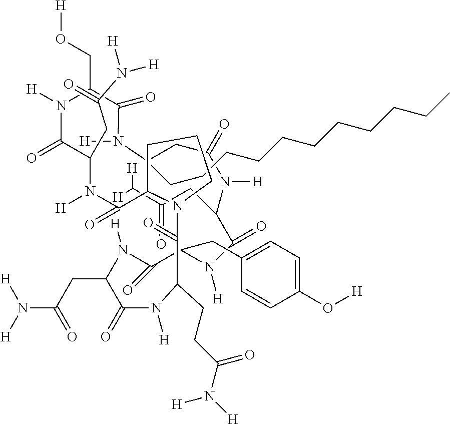 [SMILES: CCCCCCCCCCCC1CC(=O)NC(CC(C)=O)C(=O)NC(Cc2ccc(O)cc2)C(=O)NC(CC(N)=O)C(=O)NC(CCC(N)=O)C(=O)N2CCCC2C(=O)NC(CC(N)=O)C(=O)NC(CO)C(=O)N1] |
| (27) Iturin E Mycobacillin | C₄₉H₇₅N₁₁O₁₅ C₆₅H₈₅N₁₃O₃₀ | 1057_544 1527_5525 | NA NA | NA NA | 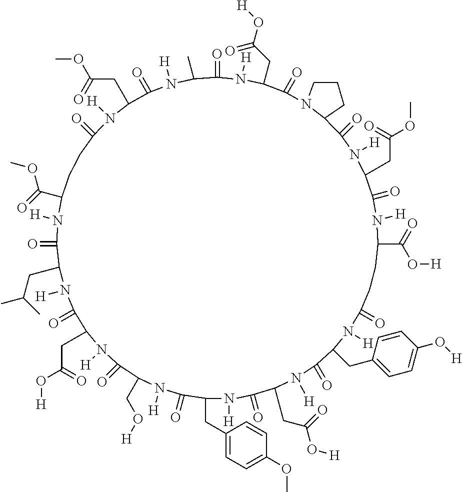 [SMILES: COC(=O)CC1NC(=O)CCC(C(=O)OC)NC(=O)C(CC(C)C)NC(=O)C(CC(=O)O)NC(=O)C(CO)NC(=O)C(Cc2ccc(OC)cc2)NC(=O)C(CC(=O)O)NC(=O)C(Cc2ccc(O)cc2)NC(=O)CCC(C(=O)O)NC(=O)C(CC(=O)OC)NC(=O)C2CCCN2C(=O)C(CC(=O)O)NC(=O)C(C)NC1=O] |
| (28) Surfactin C13 | C₅₁H₈₉N₇O₁₃ | 1007_6518 | NA | NA | 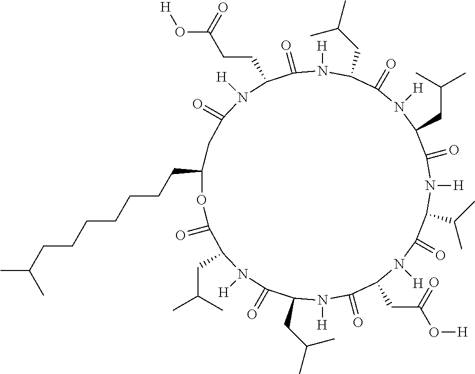 [SMILES: CC(C)CCCCCCC[C@H]1CC(=O)N[C@H](CCC(=O)O)C(=O)N[C@H](CC(C)C)C(=O)N[C@@H](CC(C)C)C(=O)N[C@H](C(C)C)C(=O)N[C@H](CC(=O)O)C(=O)N[C@@H](CC(C)C)C(=O)N[C@H](CC(C)C)C(=O)O1] |
| (29) Surfactin C14 | C₅₂H₉₁N₇O₁₃ | 1021_6675 | NA | NA | 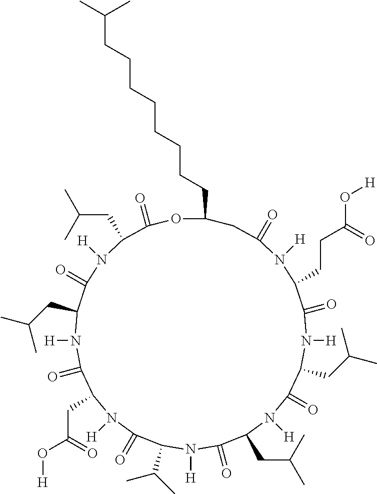 [SMILES: CC(C)CCCCCCCC[C@H]1CC(=O)N[C@H](CCC(=O)O)C(=O)N[C@H](CC(C)C)C(=O)N[C@@H](CC(C)C)C(=O)N[C@H](C(C)C)C(=O)N[C@H](CC(=O)O)C(=O)N[C@@H](CC(C)C)C(=O)N[C@H](CC(C)C)C(=O)O1] |
| (30) Surfactin C15 | C₅₃H₉₃N₇O₁₃ | 1035_6831 | C12043 | ko01054 Nonribosomal peptide structures | 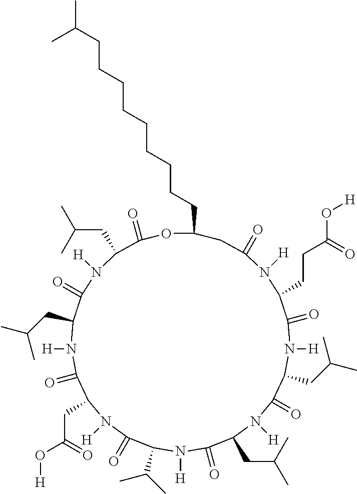 [SMILES: CC(C)CCCCCCCCC[C@H]1CC(=O)N[C@H](CCC(=O)O)C(=O)N[C@H](CC(C)C)C(=O)N[C@@H](CC(C)C)C(=O)N[C@H](C(C)C)C(=O)N[C@H](CC(=O)O)C(=O)N[C@@H](CC(C)C)C(=O)N[C@H](CC(C)C)C(=O)O1] |
| (31) Pyridines Zeatin riboside | C₁₅H₂₁N₅O₅ | 351_1543 | C16431 | ko00908 Zeatin biosynthesis | 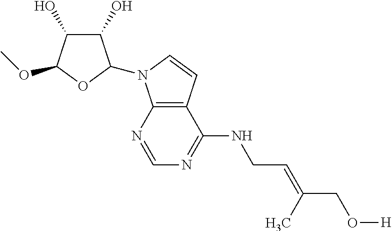 [SMILES: CO[C@@H]1OC(n2ccc3c(NC/C=C(\C)CO)ncnc32)[C@@H](O)[C@H]1O] |

In a specific embodiment, useful Bacillus methylotrophicus of the present invention are identified or defined as exhibiting one or more one or more (two or more; three or more; four or more; five or more, six of more, seven or more, eight or more, nine or more, ten or more, eleven or more or twelve) of the characteristics (3) to (14) defined above. In a specific embodiment, useful Bacillus methylotrophicus of the present invention are identified or defined as exhibiting one or more one or more (two or more; three or more; four or more; five or more, six of more, seven or more, eight or more, nine or more, ten or more, eleven or more or twelve) of the characteristics (3) to (14) defined above. In a specific embodiment, useful Bacillus methylotrophicus of the present invention are identified or defined as exhibiting one or more one or more (two or more; three or more; four or more; five or more, six of more, seven or more, eight or more, nine or more, ten or more, eleven or more or twelve) of the characteristics (3) to (14) defined above under drought conditions.

As used herein, the term “increase” or “decrease” in the context of either one of the characteristics (3) to (22) below refer to an increase or decrease, respectively of at least 5% (higher or lower, respectively) as compared to a reference characteristic in a non-bacterized plant (e.g., that of the plant in the absence of the bacterium of the present invention). In an embodiment, the increase or decrease, respectively, is of at least 10% (higher or lower, respectively), in a further embodiment, at least 15% (higher or lower, respectively), in a further embodiment, at least 20% (higher or lower, respectively), in a further embodiment of at least 30% (higher or lower, respectively), in a further embodiment of at least 40% (higher or lower, respectively), in a further embodiment of at least 50% (higher or lower, respectively), in a further embodiment of at least 60% (higher or lower, respectively), in a further embodiment of at least 70% (higher or lower, respectively), in a further embodiment of at least 80% (higher or lower, respectively), in a further embodiment of at least 90% (higher or lower, respectively), in a further embodiment of 100% (higher or lower, respectively).

Additional useful Bacillus methylotrophicus of the present invention include Bacillus methylotrophicus comprising any one of SEQ ID NOs: 1-26 (i.e. genomic sequences of the Bacillus methylotrophicus B26 strain) or 45 (16s rRNA). 16S rRNA gene sequences contain hypervariable regions that can provide species-specific signature sequences useful for identification of bacteria. In a specific embodiment, the useful Bacillus methylotrophicus of the present invention include a Bacillus methylotrophicus expressing an RNA as defined in SEQ ID NO: 45 or an sRNA substantially identical to said sequence. Additional useful Bacillus methylotrophicus of the present invention include a Bacillus methylotrophicus comprising expressing a polypeptide encoded by an exon defined by any one of SEQ ID NOs: 1-26. In another embodiment, the Bacillus methylotrophicus expresses a polypeptide that is substantially identical as that of SEQ ID NOs: 1-26. “Substantially identical” as used herein refers to polypeptides or RNAs having at least 60% of similarity, in embodiments at least 65%, 70%, 75%, 80%, 85%, 90%, 91%, 92%, 93%, 94%, 95%, 96%, 97%, 98% or 99% of similarity in their amino acid sequences. In further embodiments, the polypeptides have at least 60%, 70%, 75%, 80%, 85%, 90%, 91%, 92%, 93%, 94%, 95%, 96%, 97%, 98% or 99% of identity in their amino acid sequences (for polypeptides) or nucleotide sequences (for RNAs). In specific embodiments, the Bacillus methylotrophicus is ACC deaminase deficient.

As used herein the terms “drought conditions” refer to the set of environmental conditions under which a plant will begin to suffer the effects of water deprivation, such as decreased stomatal conductance and photosynthesis, decreased growth rate, loss of turgor (wilting), significant reduction in biomass and yield or ovule abortion. Plants experiencing drought stress typically exhibit a significant reduction in biomass and yield. Water deprivation may be caused by lack of rainfall or limited irrigation. Alternatively, water deficit may also be caused by high temperatures, low humidity, saline soils, freezing temperatures or water-logged soils that damage roots and limit water uptake to the shoot. Since plant species vary in their capacity to tolerate water deficit, the precise environmental conditions that cause drought stress cannot be generalized. Limited availability of water or drought is to be understood as a situation wherein water is or may become a limiting factor for biomass accumulation or crop yield for a non-drought resistant plant (e.g., non-bacterized plant) grown under such condition. For a plant obtained according to a method according to the present invention and grown under said condition, water may not, or to a lesser degree, be a limiting factor.

As used herein the terms “drought resistance” refers to plants that are able to modulate one or more of the below listed characteristics as follows: maintain or increase dry biomass (of shoots and/or roots), maintain or increase stomatal conductance, maintain or increase photosynthesis when subjected to drought as compared to normal/well-watered conditions. Drought resistance also refers to the ability of a plant to exhibit an increased dry biomass (of shoots and/or roots), increased stomatal conductance, increased, photosynthesis, a reduced loss of turgor or wilting, an enhanced survivability and/or a delayed desiccation when subjected to drought as compared to a plant that is not drought resistant. Differences in physical appearance, recovery and yield can be quantified and statistically analyzed using well known measurement and analysis methods. As used herein, the term “increasing” in the expression “increasing drought resistance” of a plant refers to a modulation (increase or decrease depending on the characteristic, see above) of one or more of the above characteristics of at least 5% (higher or lower, respectively) as compared to a reference drought resistance (e.g., that of the plant in the absence of the bacterium of the present invention). In an embodiment, the modulation (increase or decrease depending on the characteristic, see above) of one or more of the above characteristics is of at least 10% (higher or lower, respectively), in a further embodiment, at least 15% (higher or lower, respectively), in a further embodiment, at least 20% (higher or lower, respectively), in a further embodiment of at least 30% (higher or lower, respectively), in a further embodiment of at least 40% (higher or lower, respectively), in a further embodiment of at least 50% (higher or lower, respectively), in a further embodiment of at least 60% (higher or lower, respectively), in a further embodiment of at least 70% (higher or lower, respectively), in a further embodiment of at least 80% (higher or lower, respectively), in a further embodiment of at least 90% (higher or lower, respectively), in a further embodiment of 100% (higher or lower, respectively).

As used herein, the terms “plant growth” refers (i) an increase in the number of leaves in the plant; (ii) an increased in the plant's height; (iii) an increase in the root and/or shoot biomass; (iv) an increase in seed yield/number; (v) an increase in the total tiller number; (vi) an increased ratio of reproductive tiller/total tiller; (vii) an increased chlorophyll content leading to darker leaves; or (viii) a combination of at least two of (i) to (vii). As used herein, the term “increasing” in the expression “increasing plant growth” refers to an increase of one or more of the above characteristics of at least 5% as compared to a reference plant growth (e.g., that of the plant in the absence of the bacterium of the present invention). In an embodiment, the increase of one or more of the above characteristics is of at least 10%, in a further embodiment, at least 15%, in a further embodiment, at least 20%, in a further embodiment of at least 30%, in a further embodiment of at least 40%, in a further embodiment of at least 50%, in a further embodiment of at least 60%, in a further embodiment of at least 70%, in a further embodiment of at least 80%, in a further embodiment of at least 90%, in a further embodiment of 100%.

As used herein, the terms “well-watered” conditions for plant refer to conditions wherein water is not a limiting factor for the plant's e.g., growth and turgidity. Such conditions vary between plant species. For example, soil moisture maintained between 0.234 cm³ cm³ and 0.227 cm³ cm³ at 0-15 cm and 0.352 cm³ cm³ and 0.350 cm³ cm³ at 30-50 cm provide well-watered conditions to the plant.

The present invention shows that the inventors' Bacillus methylotrophicus (e.g., B26) is a growth enhancer and provides drought resistance to monocotyledonous plants. Bacillus methylotrophicus is a Gram-positive, rod-shape (bacillus) that can form a hard, protective endospore allowing it to withstand harsh environment, it is an obligate aerobe and can use methanol as carbon source. Bacillus methylotrophicus is part of the Firmicutes division, from the Bacilli class in the Bacillales order and Bacillaceae family.

Forms and administrations of the Bacillus methylotrophicus of the present invention.

Although the Bacillus methylotrophicus of the present invention is effective to induce tolerance when used alone (i.e. as a biologically pure strain), it may nevertheless also be used in combination with other bacteria (e.g., one or more other PGB(s) (e.g., inducing abiotic stress resistance such as salinity and/or drought resistance; and/or inducing plant growth). The present invention encompasses the use of the Bacillus methylotrophicus of the present invention as sole PGB inducing drought resistance or in combination with one or more other PGB(s).

As used herein, the terminology “biologically pure” strain is intended to mean a strain separated from materials with which it is normally associated in nature. Note that a strain associated with compounds or materials that it is not normally found with in nature, is still defined as “biologically pure”. A monoculture of a particular strain is, of course, “biologically pure.”

For the methods and uses of the present invention, it is not necessary that the whole broth culture of the strains of the invention be used. Indeed, the present invention encompasses the use of a whole broth culture of a strain of the present invention, endospores produced by such strain, dried biomass of the strains and lyophilized strains. As used herein therefore, the terminology application of the “Bacillus methylotrophicus” of the present invention refers to application of any form or part of the strain of the present invention or a combination thereof that possesses the desired ability to induce drought tolerance.

The Bacillus methylotrophicus of the present invention (e.g., B26) can take the form of a Bacillus methylotrophicus (such as whole broth culture of a strain of the present invention, endospores produced by such strain, dried biomass of the strains and lyophilized strains), a seed of a second or subsequent (up to fourth but preferably second) generation infected with the Bacillus methylotrophicus, or a composition comprising the Bacillus methylotrophicus. The Bacillus methylotrophicus of the present invention (e.g., B26), or composition thereof may be applied to soil directly prior to seeding the plant or after planting the plant (as described e.g., at Examples 14 and 15), sprayed (e.g., whole broth culture) on the plant, soil and/or on the seed of the plant. Said seed may be applied to soil directly.

There is also provided a combination of an inoculum of a strain according to the present invention and of one or more carriers to form a composition. Formulating the Bacillus methylotrophicus in a composition may increase its potential storage time and stability. Although specific compositions are disclosed herein in Example 14, many other compositions can be used in the context of the present invention.

In order to achieve good dispersion, adhesion and conservation/stability of compositions within the present invention, it may be advantageous to formulate the Bacillus methylotrophicus (such as whole broth culture of a strain of the present invention, endospores produced by such strain, dried biomass of the strains and lyophilized strains) with components that aid dispersion, adhesion and conservation/stability or even assist in the drought resistance of the plant on which it is applied. It could be formulated as a spray, granules (e.g., as that described in example 14) or as a coating for the plant seed. These components are referred to herein individually or collectively as “carrier”. Suitable formulations for this carrier will be known to those skilled in the art (wettable powders, granules and the like, or carriers within which the inoculum can be microencapsulated in a suitable medium and the like, liquids such as aqueous flowables and aqueous suspensions, and emulsifiable concentrates).

Peat-based inoculant represents a widely form of formulation but it is not a sustainable solution as peat is a non-renewable material (Xavier, Holloway et al. 2004). Alternative methods such as the encapsulation of microorganism with biopolymer are encompassed has alternative formulation methods (Xavier, Holloway et al. 2004, John, Tyagi et al. 2011). Encapsulation is the process of making a protective capsule around the microorganism. The matrix of microsphere protects the cells by providing pre-defined and constant microenvironment thus allowing the cells to survive and maintain metabolic activity for extended period of time. Microsphere can provide a control release of microorganism as well as serve as energy source for the microorganism from its degradation. Different natural polysaccharides and protein co-extruded with calcium alginate in order to form a gelled matric, matrix material such as starches, maltodextrin, gum Arabic, pectin, chitosan, alginate and legumes protein are also encompassed by the present invention (Khan, Korber et al. 2013, Nesterenko, Alric et al. 2013). Without being so limited, useful carriers for the present invention include propylene glycol alginate, powder or granular inert materials may include plant growth media or matrices, such as rockwool and peat-based mixes, attapulgite clays, kaolinic clay, mont-morillonites, saponites, mica, perlites, vermiculite, talc, carbonates, sulfates, oxides (silicon oxides), diatomites, phytoproducts, (ground grains, pulses flour, grain bran, wood pulp, and lignin), synthetic silicates (precipitated hydrated calcium silicates and silicon dioxides, organics), polysaccharides (gums, starches, seaweed extracts, alginates, plant extracts, microbial gums), and derivatives of polysaccharides, proteins, such as gelatin, casein, and synthetic polymers, such as polyvinyl alcohols, polyvinyl pyrrolidone, polyacrylates (Date and Roughley, 1977; Dairiki and Hashimoto, 2005; Jung et al., 1982). The carrier may include components such as chitosan, vermiculite, compost, talc, milk powder, gel, etc. Other suitable formulations will be known to those skilled in the art.

Without being so limited, endospores of the present invention can be incorporated in a seed coating where the material of seed coating could be as described above, e.g., biochar, peat moss, and other biopolymer carriers e.g. activated charcoal and lignosulfonate or as described in Example 14.

As used herein, the terminology “amount effective” or “effective amount” is meant to refer to an amount sufficient to effect beneficial or desired results. An effective amount can be provided in one or more administrations. In terms inducing drought resistance in plant, an “effective amount” of the microorganism of the present invention is an amount sufficient to increase drought resistance in a plant as compared to that exhibited by plant in the absence of the microorganism. In a specific embodiment, it refers to an amount of about 1×10⁸ CFU or more/plant, plant part, or area around a plant or plant part.

Plants benefiting from the B. methylotrophicus of the present invention.

In a specific embodiment, the monocotyledonous plant is of the clade commelinids. In a more specific embodiment, the commelinid plant is of the poales order. In another more specific embodiment, the poales plant is of the poaceae family (illustrated herein with Brachypodium distachyon, Phleum pratensei (timothy grass), Triticum spp. (wheat), hordeum vulgare (barley), Avena sativa (oat), Phalaris arundinacea (reed canary grass) and Bromus inermis (smooth bromegrass)). In a specific embodiment, the poaceae plant is of the pooideae subfamily (e.g., triticum spp. (wheat), hordeum vulgare (barley), Secale cereale (rye), ×Triticosecale (triticale), Avena sativa (oat), Phleum pratensei (timothy grass) and Phalaris arundinacea (reed canary grass), Bromus inermis (smooth bromegrass) and Brachypodium distachyon)). In another specific embodiment, the poaceae plant is of the ehrhartoideae subfamily (e.g., rice). In another specific embodiment, the poaceae plant is of the panicoideae subfamily (e.g., Zea mays (corn), Sorghum bicolor (sorghum), Saccharum officinarum (sugar cane), Panicum miliaceum (Proso millet); Pennisetum glaucum (Pearl millet) Setaria italica: (Foxtail millet) Eleusine coracana (Finger millet); Digitaria spp.: (Polish millet); Echinochloa spp.: (Japanese barnyard millet); Panicum sumatrense (Little Millet); Paspalum scrobiculatum: (Kodo millet) Urochloa spp (Browntop millet)).

In another more specific embodiment, the pooideae plant is of the triticeae tribe (e.g., triticum spp. (wheat), hordeum vulgare (barley), Secale cereale (rye), ×Triticosecale (triticale)). In another more specific embodiment, the pooideae plant is of the Aveneae tribe (e.g., Avena sativa (oat), Phleum pratensei (timothy grass) and Phalaris arundinacea (reed canary grass)). In another more specific embodiment, the pooideae plant is of the bromeae tribe (e.g., Bromus inermis (smooth bromegrass)). In another more specific embodiment, the pooideae plant is of the Brachypodieae tribe (e.g., Brachypodium distachyon).

As indicated above, the methods of the present invention comprises applying the B. methylotrophicus or composition thereof (i) to the plant or to a part of the plant; and/or (ii) to an area around the plant or plant part. As used herein, the term “part of the plant” or “plant part” includes shoots, leaves, etc. but also the plant's seeds. The treated seeds can be planted thereafter and grown into a plant that exhibits drought resistance properties. As used herein the terms “area around the plant or plant part” refers to the soil or plant pot prior to planting the plant seedling or seed or after having planted the plant seedling or seed.

More specifically, Bacillus methylotrophicus strain B26 is shown herein to be able to migrate from the roots to aerial parts of seedlings and behaves as a competent endophyte for representatives of the above plants. B. methylotrophicus B26 is vertically transmitted to seeds. The internal colonization of B. methylotrophicus endophytic strain B26 is shown to modulate gene expression in plants and the genes so expressed provide clues as to the effects of B26 in plants, and trigger the plant defense mechanisms to enhance resistance against drought.

Studies based on defined model systems with reduced complexity are important in elucidating the molecular mechanisms underlying Bacillus-mediated growth promoting abilities and the physiological changes enhancing their adaptation to abiotic stress (e.g., drought stress). Brachypodium distachyon is a temperate monocotyledonous plant of the poaceae grass family that is now established as the model species for functional genomics in cereal crops and bioenergy and temperate grasses like switchgrass (International Brachypodium 2010). Bachypodium is an annual, self-fertile plant with a life cycle of less than 4 months and a small nutrient requirement throughout its growth. Brachypodium distachyon can serve as a useful functional model for studying plant-endophyte interactions as it provides rapid cycling time and ease of cultivation. Many mutant accession lines and genetic web base free tools are available. Brachypodium has proven particularly useful for comparative genomics and its utility as a functional model for traits in grasses including cell wall composition, yield, stress tolerance, cell wall biosynthesis, root growth, development, and plant-pathogen interactions had been recently reported (Brkljacic et al. 2011, Mr et al. 2011). Despite these advancements in the diverse utility of Brachypodium, the usefulness of Brachypodium to study plant-bacterial endophyte interactions had not yet been explored before the present invention.

Bacillus methylotrophicus B26 was used to colonize Brachypodium distachyon as a model system to study host-endophyte interactions. The inventors examined the effect of B. methylotrophicus B26 colonization in Brachypodium and the physiological, cellular and molecular responses. First, it was investigated whether B. methylotrophicus B26 can promote vegetative and reproductive growth of Brachypodium. Second, it was confirmed that B. methylotrophicus colonizes vegetative and reproductive tissues of Brachypodium. It was also determined which role B. methylotrophicus B26 plays in a response of Brachypodium to drought conditions and which mechanisms are involved. The inventors report that a single inoculation of Brachypodium distachyon young seedlings with the strain of Bacillus methylotrophicus B26, exerts phenotypic effects throughout the whole life cycle of the plants. Besides leading to an acceleration of flowering, seed set times, senescence in bacterized plants, and structural changes in cells of intra- and intercellularly vegetative and reproductive tissues, the endophyte strain B26 does not only modulate Brachypodium drought-responsive genes in response to acute and chronic drought treatments, but also has an effect on DNA methylation and the genes that regulate said process.

Bacillus methylotrophicus B26 was also used to colonize timothy (Phleum pratense), one of the most productive C3 grass species in terms of first cut yield, that forms low aftermath growth under dry conditions (Leme\check{z}ienė, Kanapeckas et al. 2004). It is valued for its winter hardiness, good palatability and moderate nutritional feed value, and thus making it ideal for regions prone to cold winters (Bélanger, Castonguay et al. 2006). Although it is considered a winter hardy cool-season grass, it lacks heat and drought hardiness compared to many other hay grasses mainly because of shallow, fibrous roots (H. and H. 2008). In Quebec, the production of pasture, dry hay and silage make almost 65% of the diet of dairy cattle (Canada 2003), an adequate supply of quality timothy forage is essential to meet the dietary needs (Piva et al. 2013).

The effect of inoculation of the bacterial endophyte Bacillus methylotrophicus strain B26 was demonstrated on growth, water conductance, photosynthetic activity and metabolite levels (carbohydrate and amino acids) in both shoot and root tissues of timothy grass (Phleum pratense) with strain B26 and without in response to direct water deficit stress over an extended period of time. Under non-stressed conditions, strain B26 successfully colonized the internal tissues of timothy and positively impacted plant growth compared to non-inoculated plants. Exposure of inoculated plant to 8 weeks of drought stress led to significant increase in shoot and root biomass by 26.6 and 63.8%, photosynthesis and conductance by 55.2 and 214.9%, respectively compared to non-inoculated plants grown under similar conditions. Significant effects of endophyte on metabolites manifested as higher levels of several sugars, notably sucrose and key amino acids such as asparagine, the precursors of proline, glutamic acid and glutamine. The accumulation of the non-protein amino acid GABA in shoots exposed to stress and roots of stressed and not stressed plants was improved by the presence of the endophyte. Taken together, these results indicate that B. methylotrophicus B26 aids in the survival and recovery of timothy grass from water deficit and acts in part by the modification and accumulation of osmolytes in root and shoot tissues after imposition of stress.

### Example 1: Material and Methods—Growth Promotion and Endophyte Colonization

Maintenance and preparation of Bacillus methylotrophicus 826 inoculum were achieved as follows: The Bacillus methylotrophicus strain B26, previously isolated from switchgrass and fully characterized, was maintained on Luria Broth (LB) (1.0% Tryptone, 0.5% Yeast Extract, 1.0% NaCl) (Difco, Franklin Lakes, N.J., USA) with glycerol (25% final volume) and stored at −80 C. B. methylotrophicus B26 was revived on LBA (1.5% Agar) (Difco, Franklin Lakes, N.J., USA) plates. Inoculum was prepared by placing a single colony of B. methylotrophicus B26 in 250 ml of LB and incubated for 18 h at 37° C. until an OD600 of 0.7 was reached on a shaker at 250 rpm to the mid-log phase, pelleted by centrifugation, washed and suspended in sterile distilled water (Gagne-Bourque et al. 2013).

Brachypodium line, growth conditions and B. methylotrophicus inoculation were performed as follows: Growth Chamber Experiments: Brachypodium distachyon plants from the inbred line Bd21 (Brkljacic et al. 2011) were used throughout. Bd21 seeds were surface sterilized by sequentially immerging them in solutions of 70% ethanol for 30 seconds and 1.3% solution of sodium hypochlorite for 4 minutes before rinsing them three times in sterile water (Vain et al. 2008). Cone-Tainer® (Stuewe and Sons, Tanent, Or, USA) of 164 ml capacity were used to grow the plants. Prior to use, Cone-Tainers® were surface sterilized for 12 h in 0.1% NaOCl and rinsed with distilled water. Each Cone-Tainer® was filled with 1:1:1 part of sand (Quali-Grow®, L'orignal, On, Canada)/perlite (Perlite Canada, Lachine, Qc, Canada)/Agro Mix® PV20 (Fafard, Saint-Bonaventure, Qc, Canada) previously autoclaved for 3 h at 121° C. on three constitutive days. Three Bd21 sterile seeds were planted in each Cone-Tainer® and stratified at 4° C. for 7 days after which they were placed in a climatically controlled chamber (Conviron, Winnipeg, Mb, Canada) under a 16-h photoperiod with a light intensity of 150 μmoles/m²/s and a day/night temperature regime of 25/23° C. Plants were thinned to two per Cone-Tainer® after 14 days of growth, and at the same time each Cone-Tainer® received 5 ml of B. methylotrophicus B26 inoculum (10⁶ CFU/ml) or 5 ml of water (control). Bacterized and non-bacterized (control) Con-Tainers® were placed in growth chambers with identical growth parameters as previously described. Plants were harvested after 14, 28, 42 56 days post inoculation (dpi). Seeds collected from bacterized plants 56 days post inoculation were planted following the same growth conditions except that that they were not reinoculated with B26. Second generation plants were harvested after 28 days of growth.

In-vitro Culture Experiments: plant were grown in disposable culture tube 25×150 mm (V W R, Radnor, Pa., USA) in 1× Murashige and Skoog medium with 0.3% sucrose supplemented with GAMBORG′ vitamins (Sigma-Aldrich Corp., St. Louis, Mo., USA). Stratification, seed sterilization, growth conditions and inoculation were performed in a similar manner as those grown in growth chambers. Plants were inoculated with 5 ml of B. methylotrophicus B26 after 10 days of growth. Control plants received 5 ml of sterile distilled water.

Monitoring of growth parameters of Bd21 line was performed as follows: Fourteen-day-old test and control Bd21 plant groups grown in controlled growth chambers were harvested at defined phenological growth stages (Table 2) using the BBCH numerical scale (Hong et al. 2011). Harvesting was done at growth stage BBCH 13 prior to inoculation with B. methylotrophicus B26 (i.e., 0 dpi) and at the following days post inoculation (dpis) with their corresponding growth stage: 14 dpi (BBCH45), 28 dpi (BBCH55), 42 dpi (BBCH77), 56 pdi (BBCH97).

TABLE 2
Scale for phenological growth stages in Brachypodium distachyon

| Dpi* | Stage** | Description |
|---|---|---|
| 0 | BBCH13 | 3rd true leaf unfolded |
| 14 | BBCH45 | Late boot stage: flag leaf sheath swollen |
| 28 | BBCH55 | Middle of heading: half of inflorescence emerged |
| 42 | BBCH77 | Late milk |
| 56 | BBCH97 | Plant dead and collapsing |
| 70 | BBCH99 | Harvested seed |

*Days post inoculation.
**Biologische Bundesanstalt Bundessortenamt and Chemische Industrie (BBCH) growth scale (s-Yhong et al.2010).

At each harvesting time point, a minimum of fourteen Bd21 plants from seven bacterized and non-bacterized Cone-Tainers™ were monitored for root and shoot lengths, shoot and root dry weights, and number of leaves and tillers. Spikelet formation was recorded on a weekly basis while the number of seeds heads and viable seeds were recorded at the end of each experiment. Above ground nutrient content of N, P, K and Mg in vegetative above ground tissues was analyzed by Kjeldahl procedure using sulphuric acid and hydrogen peroxide digestion (Parkinson, 1975). Values were estimated in mg per gram of dry weight of tissue. All experiments were replicated two times using different growth chambers in order to control the effects of microenvironment variation.

The distribution and colonization of Brachypodium by B. methylotrophicus B26 using culture-dependent and culture-independent methods was performed as follows: To ensure that B. methylotrophicus B26 successfully and systemically colonized different plant tissues of the accession Bd21 and its intracellular spread is sustained at various Brachypodium growth stages (i.e., early and late vegetative, and reproductive stage), bacteria cell numbers and DNA copy number were determined in tissue samples and rhizosphere soil of bacterized and control Brachypodium plants. Root and leave tissues of test and control plants (first generation) of different growth stages were sampled at 14, 28 and 42 dpi, and entire young Brachypodium plants from second generation were sampled at 28 days of growth. All plants were surface sterilized as previously described (Gagne-Bourque, 2013 #397). 200 mg of tissue were pulverized to powder using a sterile mortar and pestle, serially diluted in sterile distilled water and plated on LBA. Bacterial enumeration of rhizospheric soil (1 gram) from bacterized and control Brachypodium plants was serially diluted in sterile distilled water, shaken for 30 min and plated on LBA (Skinner et al. 1952). Plates were incubated at 37° C. for 48 h. Colony forming units (CFUs) were determined and calculated to CFU per gram of fresh weight of tissue or soil. There were three biological replicates for each treatment and each replicate contained root, aerial systems or rhizospheric soil of 3 plants.

The presence of B. methylotrophicus B26 cells inside bacterized plants was also confirmed by quantitative real-time PCR (QPCR) assays. Surface sterilized plant tissues were reduced to powder in liquid nitrogen, and genomic DNA was extracted from 200 mg of powdered tissue using the CTAB method (Porebski et al. 1997) and resuspended in 100 μL of autoclaved distilled water. Genomic DNA from B. methylotrophicus B26 colonies was extracted by direct colony PCR (Woodman 2005). Briefly, single colonies were mixed with sterile distilled water, incubated at 95° C. followed by centrifugation and the supernatant was used as template DNA in conventional PCR assays.

Endophytic colonization by B26 was also confirmed by transmission electron microscopy. Fresh plant organs (roots, stems, leaves), removed from bacterized and their corresponding plants grown in vitro and in potting mix in growth chambers, were collected 5 days and 14 dpi days after inoculation, respectively. In parallel, seeds collected from the first generation plants were also collected. Sample were processed following the protocol by Wilson and Bacic, 2012 but with some modifications: fixation was carried out with 2.5% glutaraldehyde in 0.1M sodium cacodylate buffer for 7 days at 4° C., sample were washed 3 times with 0.1M sodium cacodylate washing buffer and finally an extra staining with Tannic acid 1% staining was performed after the Osmium tetroxide staining. After polymerization, capsules were trimmed and cut in section of 90-100 nm thick with an UltraCut™ E ultramicrotome (Reichert-Jung, Depew, N.Y., USA) and placed onto a 200 mesh copper grid. Samples were further stained with Uranyl acetate for 8 min, followed by Reynold's lead for 5 min. Samples were observed using a FEI Tecnai 12 120 kV transmission electron microscope (TEM) equipped with an AMT XR80C 8 megapixel CCD camera (Hillsboro, Or, USA). All reagents were purchased from Electron Microscopy Sciences, Hatfield, Pa., USA except for the Osmium tetroxide and Epon that that were supplied from Mecalab, Montreal, Qc, Canada.

B. methylotrophicus B26 DNA copy numbers in bacterized plant tissues and seeds were assessed by PCR amplification and quantification. The presence of B. methylotrophicus strain B26 within vegetative and reproductive tissues of first and second generation Brachypodium plants was confirmed by PCR using strain-specific primers (Table 3). PCR reactions along with no template controls were run under previously described conditions (Gagne-Bourque et al. 2013) using T100™ Biorad thermal cycler (BioRad, Hercules, Calif., USA. PCR products were separated on 1% agarose gels and visualized using Gel Logic 200 Imaging system from (Kodak, Rochester, N.Y., USA) under UV light.

Quantification of B. methylotrophicus B26 DNA copy number as a measure of colonization of vegetative and reproductive organs of Brachypodium was monitored at different growth stages and also in second generation plants grown from bacterized seeds using qPCR. B. methylotrophicus amplicons were purified with a QlAquick™ PCR-purification kit and cloned into pDrive (Qiagen, Venlo, Netherlands). Plasmid DNA was purified and sent for sequencing at Genome Quebec. Sequencing results were compared to the Genbank accession Ref#JN689339. The copy number of plasmid was calculated based on the concentration of purified plasmid DNA and the molecular mass of the plasmid (vector plus amplicon). A standard curve for B. methylotrophicus B26 was constructed based on the following copy numbers: 10⁹, 10⁸, 10⁷, 10⁶, 10⁵, 10⁴, 10³ and 10² which are the range of B. methylotrophicus B26 copy numbers in the different tissues of the plant. The amplification mixture reaction contained: 400 ng of template DNA, 12.5 μL of 2×SYBRII™ master mix (Agilent Technologies, Morrisville, N.C., USA), 2.5 μmol L⁻¹ of each primer and 2 μmol L⁻¹ of ROX (Agilent Technologies, Morrisville, N.C., USA) in a total volume of 25 ul. To overcome the effects of inhibitors present in the root DNA, 2.5 mg of BSA (Sigma, Oakville, On, Canada) and 3% of DMSO (Fisher, Ottawa, On, Canada) were added to each reaction. Amplification was performed in a Stratagene™ Mx3000P real-time thermal cycler (Agilent Technologies, Morrisville, N.C., USA) under the following conditions: one cycle of initial denaturation at 95° C. for 10 min, followed by 40 cycles of denaturation at 95° C. for 30 s, annealing at 50° C. for 45 s and extension at 72° C. for 45 s.

TABLE 3
List of specific and universal primers used in quantitative PCR assays

| | | | | Amplicon | GenBank for target | |
|---|---|---|---|---|---|---|
| Function | Target gene | Forward and reverse primer sequences | Primer Tm | size (bp) | gene | Query/Reference |
| Drought responsive | DHN3-like | CTCCAGCTCGTCCGAGGAT (SEQ ID | 58.8 | 112 | XM_003574949.1 | ABO14458.1 |
| | | NO: 27) | | | | |
| | | AGCCATGTGCTGCTGGTTAT (SEQ ID | 57.2 | | | |
| | | NO: 28) | | | | |
| | LEA-14-A-like | TCGACTACGAGATGCGGGTC (SEQ ID | 58.7 | 115 | XM_003565767.1 | NP_171654 |
| | | NO: 29) | | | | |
| | | CAGAAGATGTCGGAGAGCGTG (SEQ | 57.6 | | | |
| | | ID NO: 30) | | | | |
| | DREB2B-like | AGCTGACGACCTCTTTGAGC (SEQ ID | 57.2 | 110 | XM_003568607.1 | BAA36706 |
| | | NO: 31) | | | | |
| | | CTACCGGGTCAGCTTCCATC (SEQ ID | 57.4 | | XM_003568608.1 | |
| | | NO: 32) | | | | |
| Methyltransferases | MET1B-like | AGACCTCCCACCTCTCTTGG (SEQ ID | 58.2 | 101 | XM_003561293.1 | NP_199727.1 |
| | | NO: 33) | | | | |
| | | GCTCAGTCTCCAATTGGCCT (SEQ ID | 57.5 | | | |
| | | NO: 34) | | | | |
| | CMT3-like | GATCGCGTGCAACAGATTCC (SEQ ID | 56.8 | 110 | XM_003571630.1 | NP_177135.1 |
| | | NO: 35) | | | | |
| | | ACTCGCTGAACTTCTGGGTC (SEQ ID | 56.9 | | | |
| | | NO: 36) | | | | |
| | DRM2-like | AAGAAGACAGCTCAACTGCGTGC | 60 | 77 | XM_003575408.1 | NP_196966.2 |
| | | (SEQ ID NO: 37) | | | | |
| | | TTGCAAGAGCACATTGGATCCGC (SEQ | 60.5 | | | |
| | | ID NO: 38) | | | | |
| Internal Standard | Bradi18S | GAAGTTTGAGGCAATAACAGGTCT | 55.3 | 131 | XM_003579769.1 | Colton-Gagnon et |
| | | (SEQ ID NO: 39) | | | | al., 2013 |
| | | ATCACGATGAATTTCCCAAGATTAC | 53.5 | | | |
| | | (SEQ ID NO: 40) | | | | |
| | SamDC | AGCGAGTCGACGATACCCTT (SEQ ID | 57.9 | 190 | DV482676 | Hong et al., 2008 |
| | | NO: 41) | | | | |
| | | TGCTAATCTGCTCCAATGGC (SEQ ID | 55.4 | | | |
| | | NO: 42) | | | | |
| Quantification of B. | 16s ITS rRNA | CAAGTGCCGTTCAAATAG (SEQ ID NO: | 48.7 | 565 | JN_689339 (SEQ ID | Gagne-Bourque et al., |
| methylotrophicus | | 43) | | | NO: 45) | 2013 |
| | | CTCTAGGATTGTCAGAGG (SEQ ID NO: | 48.3 | | | |
| | | 44) | | | | |

Standard curves and no template controls were run with each plate. All samples were performed in triplicate technical runs. Amplification results were expressed as the threshold cycle (Cₜ) value and converted to copy numbers by plotting the Cₜ values against the standard curve. The coefficient of variation was calculated for each sample to ensure repeatability of amplification. Samples with a coefficient of variation above 1 had their outliers removed.

RNA extraction and cDNA synthesis were performed on aerial parts of four bacterized and not bacterized plants which were pooled and reduced to fine powder in liquid nitrogen. Total RNA was extracted from 100 mg of powder using the Total RNA Mini Kit, plant (Geneaid, Shanghai, China) following the manufacturers protocol. All RNAs were treated with DNase I (Qiagen, Venlo, Netherlands) to remove genomic DNA (Qiagen, Venlo, Netherlands). cDNA was synthesized using the iScript™ cDNA Synthesis Kit (BioRad, Hercules, Calif., USA). The resulting cDNA samples were diluted to a final concentration of 2.5 ng/μL for QPCR, and stored at −20° C. Parallel reactions were run for each RNA sample in the absence of reverse transcriptase (no RT control) to assess any genomic DNA contamination.

### Example 2: The Inoculation of Bacillus methylotrophicus Strain B26 Improved Production of Biomass and Seeds

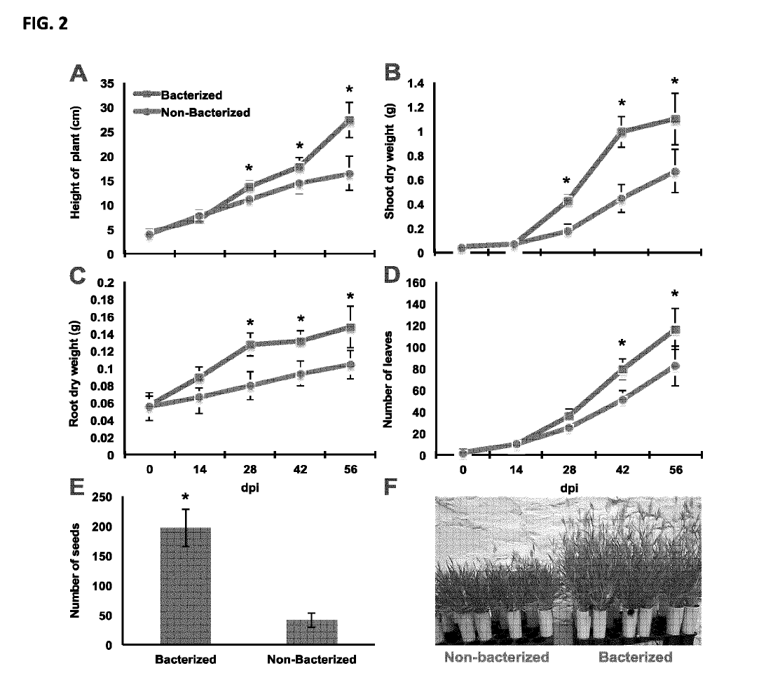

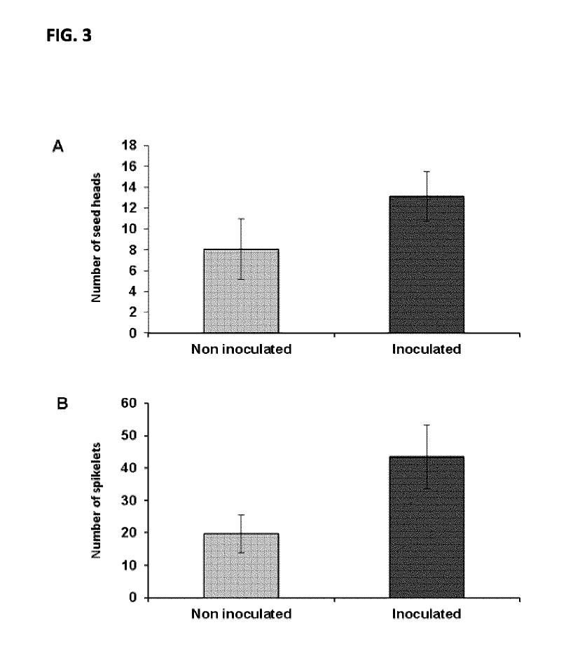

The model plant Brachypodium distachyon provides many advantages for genomics in grasses including its small genome and rapid life cycle, public databases for genome sequences and gene information. In the present application, the inventors sought to examine the ability of B. methylotrophicus B26 to promote growth of Brachypodium in growth chamber experiments. Bacterized Brachypodium plants developed faster relative to non-bacterized plants and showed a significant and steady increase in plant growth at 28 dpi (P<0.05). At the reproductive stage (56 dpi), significant growth promotion with a 65.8%, 63.8%, 42.3% and 41.5% increases in plant height (FIG. 2 A), shoot (FIG. 2 B) and root (FIG. 2 C) dry biomass and number of leaves (FIG. 2 D), respectively was observed, suggesting that B26 behaved as a plant growth promoting bacterium in Brachypodium (FIG. 2F). Bacterized plants produced 64% more seed heads than control plants (FIG. 3), indicating that more tillers became reproductive in bacterized plants. Notably, bacterized plants produced 121% more spikelets (FIG. 3) resulting in approximately 377% increase in seed yield (FIG. 2 E). Concentrations of N, P, K and Mg in above ground tissues of bacterized plants were significantly lower at 42 dpi (Table 4), indicating that the growth promoting ability was not related to increase in nutrients.

TABLE 4
Nutrient analysis of above ground of control (C) and bacterized
Brachypodium with B. methylotrophicus 26 (B+)

| | Above Ground Tissues* | | | | |
|---|---|---|---|---|---|
| | Nutrients (mg/g) | | | | |
| | Days post | Nitro- | | | |
| Treat- | inoculation | gen | Phosphorus | Potassium | Magnesium |
| ment | (dpi) | (N) | (P) | (K) | (Mg) |
| B+ | 28 | 32.83a | 7.98a | 29.47a | 1.59a |
| Control | 28 | 38.38a | 7.60a | 3.07a | 1.75a |
| B+ | 42 | 15.75b | 4.39b | 16.39b | 0.85b |
| Control | 42 | 21.52a | 6.55a | 21.92a | 1.11a |

Tissues were harvested 28 and 42 days post inoculation (dpi) with B. methylotrophicus. Analysis data were subjected to one-way ANOVA. The significance of the effect of the treatments was determined via Tukey HSD with a magnitude of the F-value (P=0.05). Treatments were tested in pairwise comparison for each time point dpi

### Example 3: B. methylotrophicus Strain B26 Successfully and Stably Colonize Vegetative and Reproductive Organs of Brachypodium distachyon

Colonization demonstrated by bacterial counts and CFU. The success of internal and systemic colonization of Brachypodium distachyon by B. methylotrophicus B26 was confirmed by culture-dependent and independent methods. Re-isolation and quantification of B. methylotrophicus strain B26 by the plating method in different surface-sterilized tissues of first and second generations of Brachypodium plants after soil drench treatment with B. methylotrophicus clearly demonstrate that B. methylotrophicus B26 can form sustaining endophytic populations in roots, shoots and seeds as well as in the soil around the roots of Brachypodium (Table 5 below). Following rhizosphere colonization of Brachypodium, bacterial counts within root tissue changed with the plants growth stage, while numbers of CFUs in shoots stabilized over the last two growth stages (BBCH 55 and BBCH97). However, population numbers in shoots were consistently higher than in roots indicating that there was successful translocation from roots to shoots. CFU numbers in rhizosphere soil remained stable over time. Moreover, vegetative tissues of the Brachypodium young plants (BBCH45) that originated from seeds of the first generation sustained similar population numbers to those from the first generation for the corresponding growth stage (Table 5 below). Population numbers in Brachypodium seeds were lower by a factor of 10 compared to other tissues. Rhizosphere soil and surface sterilized tissues of control plants did not yield cultivable bacterial colonies.

Surface-sterilized tissues of 1^{st} and 2^{nd} generations of Brachypodium clearly demonstrate that B. methylotrophicus B26 can form sustaining endophytic populations in all tissues as well as in the rhizosphere. Bacterial counts (CFU) in shoots were consistently higher than in roots. Brachypodium vegetative tissues originating from seeds of the 1^{st} generation sustained similar population numbers to those from the 1^{st} generation for the corresponding growth stage.

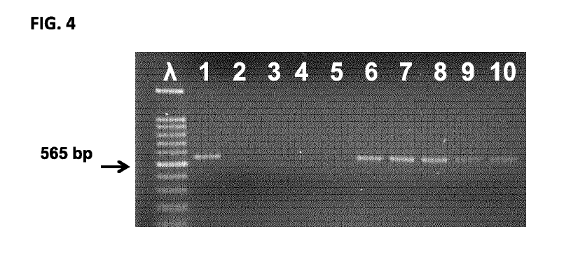

Colonization demonstrated by QPCR. Additionally, the presence of B. methylotrophicus B26 in different tissues of Brachypodium was confirmed by QPCR in bacterized plants (FIG. 4). An amplicon with the expected product size of 565 bp was successfully amplified using species-specific primers for B. methylotrophicus B26 from DNA extracted from each tissue type (FIG. 4). Non-bacterized tissue samples tested negative for the presence of B. methylotrophicus B26. (FIG. 4). Absolute quantification by QPCR of B. methylotrophicus B26 copy numbers sustained the same numbers in the root at all growth stages and a small decrease in shoot tissue, with 10 times more copy in Brachypodium shoots compared to roots (FIG. 4). Copy numbers in seeds of B. methylotrophicus B26 were the lowest of all tissues tested. Second generation plant tissue showed the highest concentration of endophyte in the root and a lower amount in the shoot than in the bacterized plant at corresponding growth stages.

Absolute quantification of B. methylotrophicus B26 copy numbers by qPCR sustained similar numbers in roots at all growth stages; a small decrease in shoots; and lower numbers in seeds. Second generation plant tissues had the highest concentration of endophyte in roots and a lower amount in shoots compared to 1^{st} generation.

TABLE 5
Dynamics of B. methylotrophicus B26 in the host plant. Colony Forming Units (CFU) and
DNA copy number of B. methylotrophicus B26 in roots, shoots, seeds and rhizospheric soil.
Uppercase letter represent difference in between time point of the same tissue/soil and
lowercase represent difference between different tissues at the same time point.

| | | Log CFU/g Fresh Weight | | | | Copy/100 mg Tissue | | |
|---|---|---|---|---|---|---|---|---|
| | | Tissue | | | | Tissue | | |
| Growth stage | | Root | Shoot | Seed | Soil | Root | Shoot | Seed |
| | Day post inoculation | | | | | | | |
| | (dpi) | | | | | | | |
| BBCH45 | 14 | 3.68 Ca | 3.62 Ba | | 3.68 A | 2.06 × 10⁵ Ac | 3.24 × 10⁶ Aa | |
| BBCH55 | 28 | 3.86 Ab | 3.91 Aa | | 3.67 A | 2.07 × 10⁵ Ab | 3.55 × 10⁶ Aa | |
| BBCH97 | 56 | 3.76 Bb | 3.92 Aa | | 3.63 A | 1.82 × 10⁵ Ab | 1.63 × 10⁶ Ba | |
| BBCH99 | 70 | | | 2.47 | | | | 1.45 × 10⁵ |
| | Second generation plant | | | | | | | |
| | Age in days (D) | | | | | | | |
| BBCH45 | 28 | 3.60 Aa | 2.76 Bb | | | 1.97 × 10⁶ Aab | 3.52 × 10⁵ Bbc | |

### Example 4: Effect of Systemic Colonization of Plants by B. methylotrophicus B26 on Immune Response

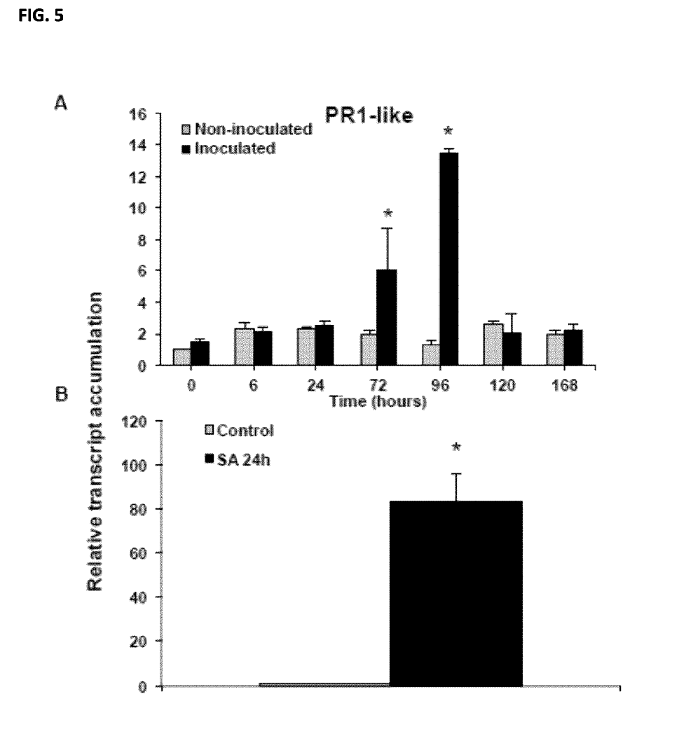

The ability of bacterial endophytes to colonize plants is a complex process requiring resistance to plant defence systems. To assess therefore whether the systemic colonization of Brachypodium distachyon by B. methylotrophicus B26 triggers an immune response, the inventors monitored the transcript accumulation levels of the pathogenesis-related PR1 gene in bacterized and non-bacterized plants using qRT-PCR. Since the PR1 gene is not fully characterized in the Brachypodium model, the inventors first sought to determine if an exogenous application of salicylic acid (SA) could trigger a transcripts accumulation of the selected Brachypodium PR1-like gene (FIG. 5 B). As expected, Brachypodium plants sprayed with 5 mM solution of SA had 84 times more PR1-like transcripts than control plants at 24 hours after treatment. The inventors then monitored the PR1-like transcript accumulation patterns during the early colonization stages of Brachypodium plants by B. methylotrophicus B26. Bacterized plant showed a 6-fold increase of PR1-Like transcript accumulation at dpi 3 and 4 followed by a decrease to basal levels at dpi 5 and 7 (FIG. 5A). Taken together this result suggests that Bacillus methylotrophicus B26 is mostly perceived as a non-pathogenic bacterium during the systemic colonization of Brachypodium distachyon.

### Example 5: Material and Methods—Water Deficit Stress

Growth conditions and drought stress were assessed as follows: To investigate whether B. methylotrophicus B26 confer drought tolerance to Bd21, two types of drought stress were applied: chronic and acute water deficit stresses. Studies on the effect of chronic water deficit stress were carried out on Brachypodium seedlings stratified and germinated as previously described but planted in 10×10 cm pots (ITML, Brantford, On, Canada) filled with sterilized Agro Mix® G6 (Fafard et frères, Qc, Canada) with three plants per pot. Plants were grown under the same growth chamber conditions and were inoculated or not with B. methylotrophicus B26 as previously described.

Chronic water deficit stress was conducted on test and control plants at dpi 28 by withholding water from the bacterized plants while control plants were watered with 50 ml of sterile water 3 times per week. Plants were harvested on day 0, 5 and 8 of withholding water and leaf tissue was immediately frozen in liquid nitrogen and prepared for transcript accumulation analysis for drought responsive genes and starch and sugar content analysis. A total of 3 replicates per treatment were sampled at each time point. A replicate consisted of 3 plants. The experiment was repeated twice.

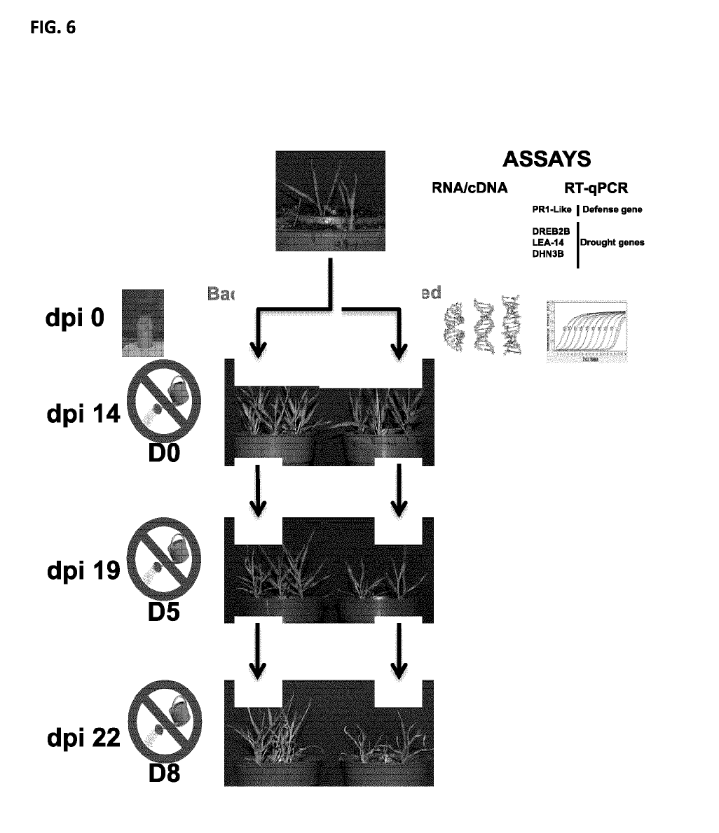

Acute water deficit stress was applied on young Bd21 seedlings grown in vitro cultures at 3 pdi, by uprooting the plants from the medium and left on an open bench for 1 hour before being flash frozen in liquid nitrogen. The entire plants were sampled, flash frozen in liquid nitrogen and subjected to transcript accumulation analysis. A total of 4 replicates per treatment were sampled and the experiments were repeated three times. FIG. 6 shows the methodology used herein to subject Brachypodium distachyon to chronic water stress.

Gene identification and primer design were performed as follows: Using Arabidopsis thaliana protein sequences as query, identified Brachypodium distachyon's orthologs of the following drought-responsive encoding genes; DREB2B, LEA-14, DHN3 and the DNA methyltransferase encoding genes MET1B, CMT3, and DRM2 were used. The drought responsive gene, DHN3-like was identified using a DHN3 protein sequence from Hordeum vulgare (Table 3). Primer sets were designed using Primer BLAST for specificity and synthesized by Integrated DNA Technologies, Inc. (Coralville, Iowa, USA). The primer pairs for 18S Ribosomal RNA and SamDC have been used previously (Colton-Gagnon et al. 2013; Hong et al. 2008).

RT-QPCR data analysis and relative quantification of stress-responsive genes and PR1 were performed as follows: Quantitative real-time PCR was performed using a CFX Connect Real Time system (BioRad, Hercules, Calif., USA), using Sso-advanced SYBR green Supermix (BioRad, Hercules, Calif., USA). Amplification was performed in an 11 μl reaction containing 1×SYBR Green master mix, 200 nM of each primer, 10 ng of cDNA template. The PCR thermal-cycling parameters were 95° C. for 30 seconds followed by 40 cycles of 95° C. for 5 seconds and 57.5° C. for 20 sec (Table 3). Three technical replicates were used and the experiment was repeated three times with different biological replicates. Controls without template were included for all primer pairs. For each primer pair, two reference genes (18S and SamDC) were used for normalisation. The RT-qPCR data was analysed following the Livak method (Livak and Schmittgen 2001).

Starch and water-soluble sugar analyses were performed as follows: one hundred (100) mg of freeze-dried ground leaf tissues of bacterized or not plants subjected to drought or not were pooled and reduced to fine powder in liquid nitrogen. Soluble sugars were extracted with methanol/chloroform/water solutions and analyzed as described in Piva et al 2013 using a Waters ACQUITY Ultra Performance Liquid Chromatography (UPLC) analytical system controlled by the Empower II software (Waters, Milford, Mass., USA). Peak identity and quantity of raffinose, sucrose, glucose and fructose were determined by comparison to standards. Total starch was extracted from the non-soluble residue left after the methanol/chloroform/water extraction and quantified as a glucose equivalent following enzymatic digestion with amyloglucosidase (Sigma A7255; Sigma-Aldrich Co., St. Louis, Mo.) and colorimetric detection with p-hydrobenzoic acid hydrazide method of (Blakeney, 1980 #460).

DNA methylation analyses were performed as follows: A global DNA methylation assay was performed using the Imprint® Methylated DNA Quantification Kit (Sigma-Aldrich Corp., St. Louis, Mo., USA) according to the manufacturer's recommendations with 200 ng/μL of DNA per well. Each sample was measured in technical quadruplicate using a 680 Microplate reader (BioRad, Hercules, Calif., USA). Genomic DNA was extracted following the methods mention previously.

Statistical analysis was performed as follows: All experimental data were subjected to statistical analyses by performing one-way ANOVA using the JMP 10.0 software (SAS Institute, Cary, N.C., USA). The significance of the effect of the treatments was determined via Tukey HSD with a magnitude of the F-value (P=0.05). In the case of repeated experiment trials results were tested using Levene's test for equality of variance (P=0.05) and pooled if permitted.

### Example 6: B. methylotrophicus Bacterized Plant Tolerance to Water-Deficit Stress

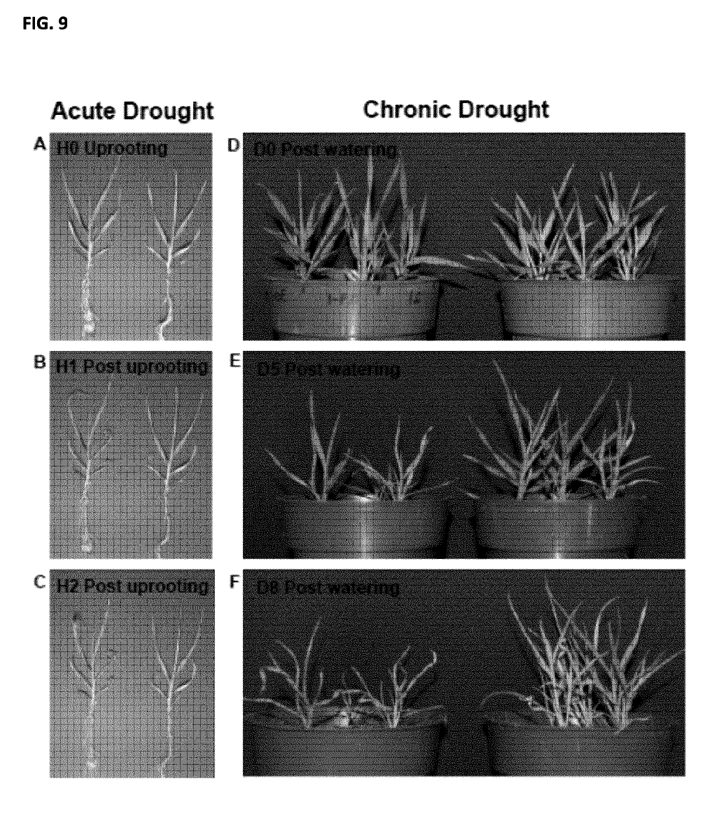

Bacterized Brachypodium plants were more tolerant to water-deficit stress as demonstrated as follows: An unexpected observation that bacterized Brachypodium plants uncared-for for several days were doing notably better than the non-bacterized ones prompted the inventors to evaluate the contribution of B. methylotrophicus B26 to the plant's capacity to tolerate drought. An initial assay, consisted of an acute water-deficit stress applied by uprooting young non-bacterized and bacterized Brachypodium seedlings grown in vitro from the medium and by leaving them on an open bench for 1 h. After this acute drought treatment, the leaf tips of non-bacterized plants showed clear signs of wilting while bacterized plants looked mostly unaffected (FIGS. 9 A to C). A chronic drought treatment was performed in a soilless potting media with non-bacterized and bacterized plants at 28 dpi by withholding water for 5 and 8 days. Again, bacterized plants showed less signs of wilting and ultimately died later than non-bacterized plants (FIGS. 9 D to F).

### Example 7: Gene Expression During Drought Conditions in the Presence of B. methylotrophicus B26

Plant genes may be modulated by the presence of B. methylotrophicus B26, and the genes so expressed provide clues as to the effects of endophytes in plants.

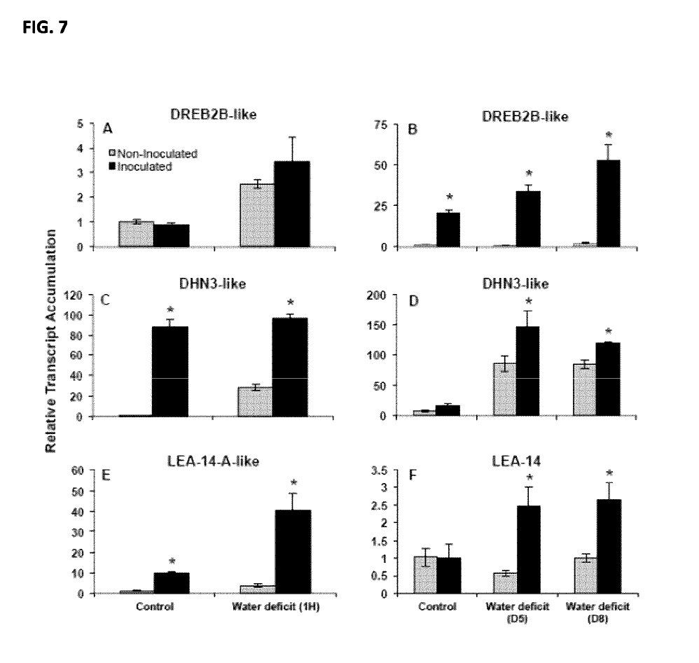

B. methylotrophicus strain B26 modulated the expression of the plant's drought responsive genes. To determine the role of B. methylotrophicus B26 in the plant's drought-response mechanism, Brachypodium genes with high sequence similarities to genes previously characterized to play active roles in the drought-stress response of plants (Table 3) were selected and quantitative real-time PCR assays were conducted to monitor their transcript accumulation profiles. Bacterized and non-bacterized Brachypodium plants grown in vitro under control conditions displayed similar accumulation profiles of the DREB2B-like transcript (FIG. 7A). However, a one-hour acute drought treatment triggered increases in DREB2B-like transcripts accumulation of respectively 2.5 fold and 3 fold in non-bacterized and bacterized Brachypodium plants (FIG. 7A). On the other hand, bacterized plants grown under normal conditions in soilless potting media had 14-times more DREB2B-like transcript levels than non-bacterized plants grown in similar conditions (FIG. 7B). In addition, chronic drought conditions, obtained by withholding water for 5 and 8 days, caused significant increases in the levels of DREB2B-like transcripts in bacterized plants but not in non-bacterized plants (FIG. 7B).

The transcription factor DREB2B has been shown to act upstream of structural proteins such as dehydrins in Arabidopsis and other plants. Changes in the expression profiles were monitored in response to acute and chronic drought stresses of two Brachypodium genes with high sequence similarities to the dehydrins DHN3 and LEA-14-A. Compared to non-bacterized Brachypodium plants, a 70-fold accumulation in DHN3-like transcripts was observed in bacterized control plants grown in vitro (FIG. 7C) while no significant difference was observed for plants grown in soilless potting mix growth media (FIG. 7D). The application of an acute drought treatment triggered a 20-fold accumulation of the DHN3-like transcript in non-bacterized (non-inoculated) plants as compared to that in its corresponding control plant but had no significant effect (as compared to that in its corresponding control plant) on the already high accumulation of this transcript in bacterized (inoculated) plants (FIG. 7C). Conversely, chronic drought treatments of either five or eight days triggered a 85-fold accumulation of the DHN3-like transcript in bacterized plants and a 9-fold accumulation of the same messenger in non-bacterized plants (FIG. 7D). A similar transcript accumulation pattern was also observed for the LEA-14-A-like gene (FIGS. 7E and F).

### Example 8: Structural Changes in Colonized Plant Tissues

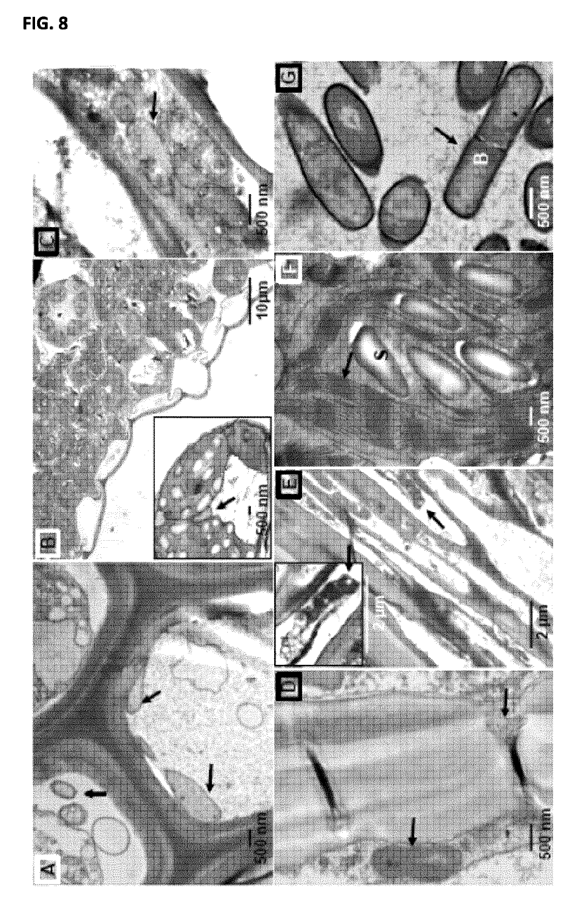

Structural changes in colonized plant tissues were assessed as follows: The interaction of B. methylotrophicus B26 with Brachypodium was followed using transmission electron microscopy (TEM). The inventors examined the internalization and distribution of B. methylotrophicus B26 within roots, leaves, stems and seeds of bacterized (14 and 28 dpi) Brachypodium plants grown under gnobiotic and greenhouse conditions, (FIG. 8). TEM analysis of tissue sections confirmed the presence of B. methylotrophicus B26 cells inside xylem tissue of roots (FIG. 8A), mesophyll cells and bundle sheath of leaves (FIGS. 8 B and C) stems (D), in seeds (FIG. 8E) and in choloroplast of a leaf bundle sheath cell (FIG. 8F). The morphology and size of B. methylotrophicus B26 cells inside plant tissues are identical to B. methylotrophicus B26 cells grown in pure culture (FIG. 8G). Mesophyll cells close to leaf veins of bacterized plants show substantial accumulation of unusually large starch granules in the chloroplast interspersed in the stroma and sometimes separating the thylakoids (FIG. 8F). However, the outer membranes of the plastids were still intact (FIG. 8F, arrow). Mesophyll cells of non-bacterized leaf blades had little or no starch granules (data not shown). Sections of control samples were devoid of bacterial cells (data not shown), suggesting no indigenous colonization.

### Example 9: Carbohydrate and Starch Accumulation in B. methylotrophicus Bacterized Plant in Drought Stress Conditions

Osmoregulation in plants via accumulation of soluble sugars like glucose, sucrose and fructose is a known mechanism for maintaining homeostasis in plants under drought stress conditions (Wang et al. 2010) and their metabolism play a significant role in drought and cold stress tolerance (Valliyodan et al 2006). Similarly, increased biosynthesis rates of soluble sugars in corn inoculated with a plant growth promoting pseudomonas exposed to drought stress was also reported (Figueiredo et al 2008).

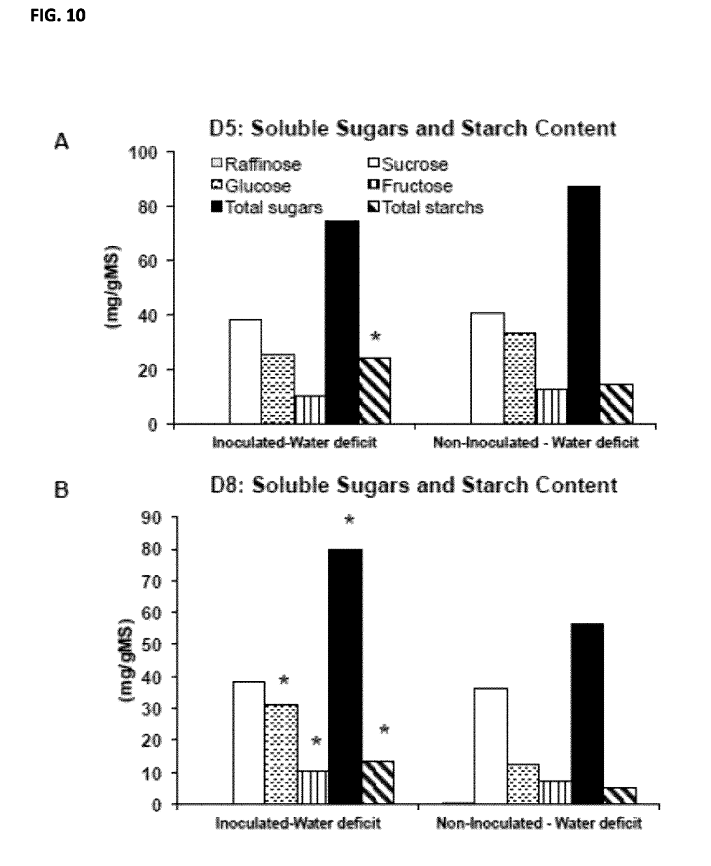

Bacillus methylotrophicus B26 stimulated carbohydrate and starch accumulation under drought stress conditions. Leaf tissues of B. methylotrophicus inoculated and non-inoculated Brachypodium were analyzed for carbohydrate and starch at the end of 5 and 8 days of chronic drought stress. Stressed inoculated plants had almost 2-fold and 3-fold increase of total starch at the end of 5 and 8 days of drought stress respectively, compared to stressed but not-inoculated plants (FIG. 10). Drought stress did not have any influence on the amount of individual and total sugars of inoculated and non-inoculated plants after 5 days of stress. Inoculated plants exposed to stress for 8 days however had 1.4-fold more of total soluble sugars, and also 2.9-fold and 1.4 fold increases in glucose and fructose, respectively.

This increase in total soluble sugar and starch in above ground tissues of Brachypodium-inoculated plants could compensate the drought effects and improve plant developments through among others, the enhanced production of soluble sugars resulting in a better absorption of water and nutrients form the soil.

The latter observation ties well with copious accumulation of large starch granules in the stroma of chloroplasts of leaf bundle sheath cells of bacterized plants relative to control plants. The starch packing had no visible effects on the grana. To the best of the inventors' knowledge, this extensive loading of leaf chloroplasts with starch in response to bacterial endophytic colonization has not been reported. In addition to increased availability of starch as reserve to plants under stress, this modification could result in the enhancement of nutrient flow to bacterial cells.

### Example 10: DNA Methylation in B. methylotrophicus Bacterized Plant in Drought Stress Conditions

Drought conditions have been shown to naturally induce DNA methylation changes in plants that in turn increase the plant resistance toward the stress by allowing the expression of protective genes involved in the drought response.

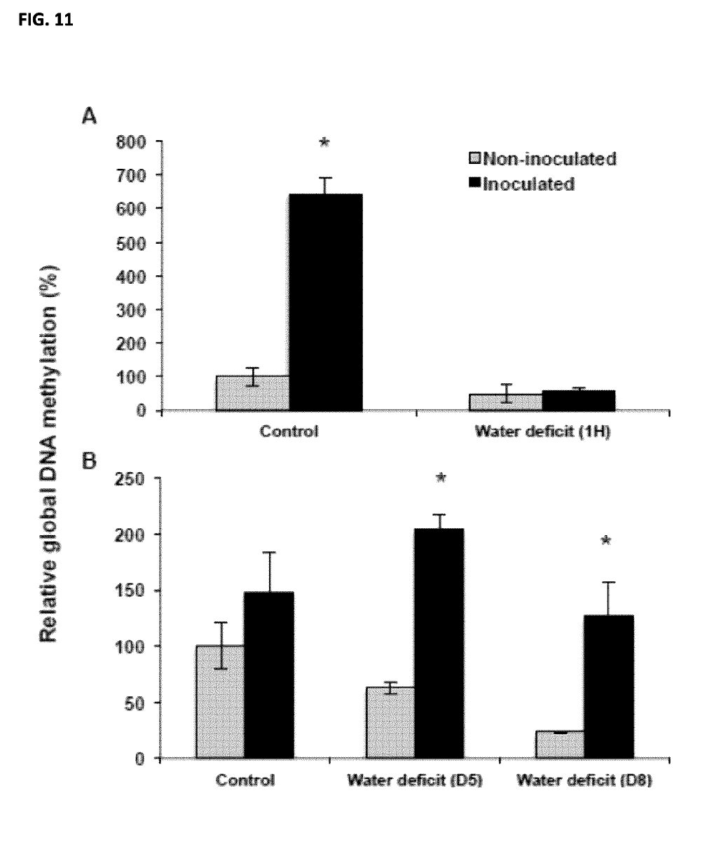

Bacillus methylotrophicus B26 triggered changes in DNA methylation in Brachypodium. The changes in transcript accumulation observed in FIG. 11 suggest that B. methylotrophicus B26 triggered important chromatin changes in the host plant. Whole plant DNA methylation was measured in bacterized and non-bacterized Brachypodium plants under normal and drought conditions (FIG. 11). B. methylotrophicus B26 triggered 6-fold and 1.5-fold increases in global DNA methylation in plants grown under normal conditions either in vitro (FIG. 11A) or in soilless potting mix (FIG. 11B). On one hand, after one hour of acute drought treatment, the global DNA methylation levels observed in in vitro bacterized plants returned to those of non-bacterized plants while this treatment had no effect on the global DNA methylation levels of non-bacterized plants (FIG. 11A). On the other hand, clear reductions in global DNA methylation were observed in non-bacterized plants after five and eight days of chronic drought treatment (FIG. 11B). These reductions were not observed in bacterized plants exposed to similar drought stress conditions since an overall increase in whole plant DNA methylation pattern was observed after five days of chronic drought. These results suggest that B. methylotrophicus can affect the epigenetic regulation of Brachypodium distachyon before and during drought stress.

### Example 11: DNA Methyltransferases Expression in B. methylotrophicus Bacterized Plant in Drought Stress Conditions

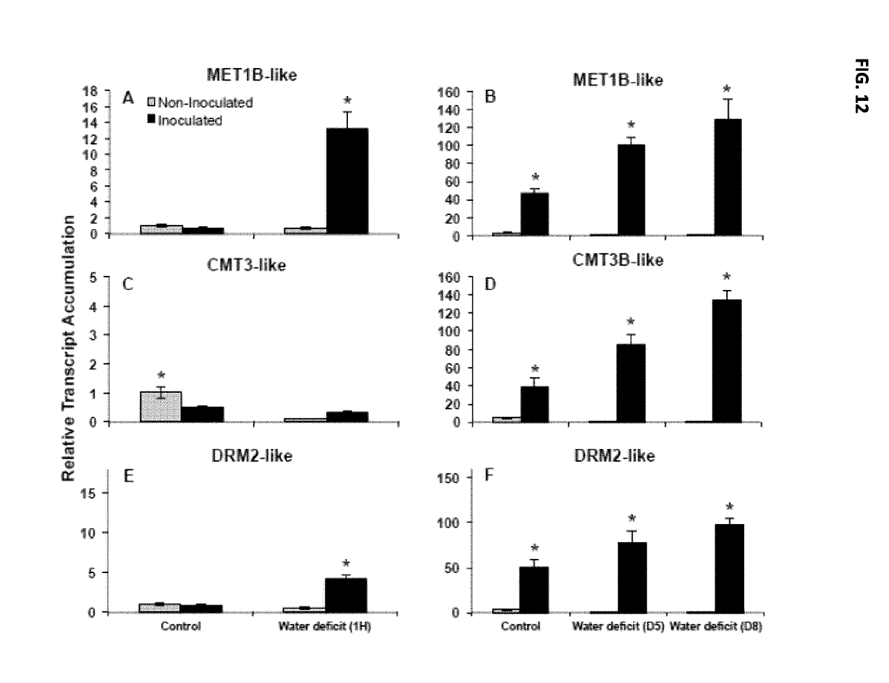

The drastic changes in global DNA methylation observed upon bacterization of Brachypodium suggest the involvement of several DNA methyltransferases in regulating that process. Changes of transcript accumulation were monitored in bacterized and non-bacterized plants in response to drought for three DNA methyltransferases: MET1B-like, CMT3-like and DRM2-like. As shown in FIG. 12, drought treatments had very little impact on the transcript accumulation of the three DNA methyltransferases tested in non-bacterized plants either grown in vitro (FIGS. 12A, C, E) or in soilless potting mix (FIGS. 12 B, D, F). Similarly, bacterized Brachypodium plants grown in vitro under control conditions did not show significant differences in accumulation of DNA methyltranferase transcripts (FIGS. 12A, C and E). On the opposite, bacterized Brachypodium plants subjected to one hour of acute drought stress showed increased MET1B-like and DRM2-like transcript accumulations (FIGS. 12A and E). In addition, bacterized plants grown in soilless potting mix under control conditions accumulated more of the three DNA methyltransferase transcripts than non-bacterized plants (FIGS. 12B, D and F). Moreover, chronic drought conditions for five and eight days further increased the accumulation of these transcripts in bacterized plants but not in non-bacterized plants (FIGS. 12B, D and F).

### Example 12: Material and Methods—Bacillus methylotrophicus B26 for Promoting Growth in Crop Plants

Poaceae plant growth conditions: Seeds from corn, wheat, barley, oat, timothy, smooth bromegrass and reed canarygrass were grown in a growth chamber at 22° C. under a 12 h/12 h of light/dark cycle, water with 300 ml of water 3 times per week and fertilize every 14 days with 300 ml per pots of a solution of 2 g/liter of all-purpose fertilizer 20-20-20 (Plantprod, Laval, Québec). Plants were grown in 15*20 cm pots filled with Agromix® (Plantprod, Laval, Quebec). 5 plants were grown per pot, except for corn were 2 plants was used. 10 plants for each species per treatment was use.

Maintenance and preparation of Bacillus methylotrophicus 826 inoculum: Bacterial endophytes Bacillus methylotrophicus B26 were grown in LB broth for 18 h to the mid-log phase, pelleted by centrifugation, washed and suspended in sterile distilled water. 14 days after planting, each plant received 5 ml of water containing 10⁵ CFU ml⁻¹ of bacteria. Seedlings receiving autoclaved distilled water served as controls.

After 91 days of growth all the plants were harvested, dried for 4 days at 55° C. and dry weight of all plants was recorded and statistically compared to control treatment.

Statistical analysis was performed as follows: Data were analyzed by one-way ANOVA using the JMP 10.0 software (SAS Institute, Cary, N.C., USA). The significance of the effect of the treatments was determined by the magnitude of the F-value (P=0.05) and difference in treatment was determined using the Tukey HSD test (P=0.05).

### Example 13: Effect of Bacillus methylotrophicus B26 on Growth in Crop Plants

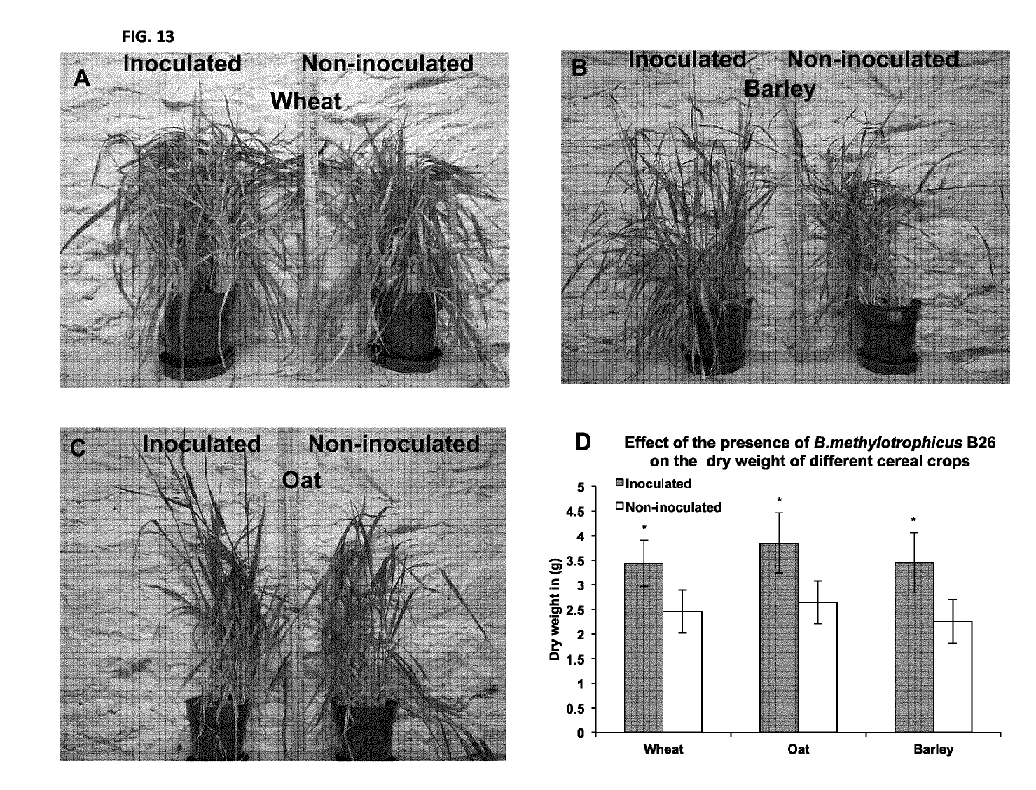

The experiment was designed to test the ability of bacterial endophytes, Bacillus methylotrophicus B26, to colonize and affect growth in different crop types of the Poaceae family. The difference in growth between inoculated and non-inoculated plants of wheat (FIG. 13A), barley (FIG. 13B), and oats (FIG. 13C) was assessed visually at harvest, and by the respective dry mass of said plants (FIG. 13D). The differences in all three species between inoculated and non-inoculated plants were statistically significant.

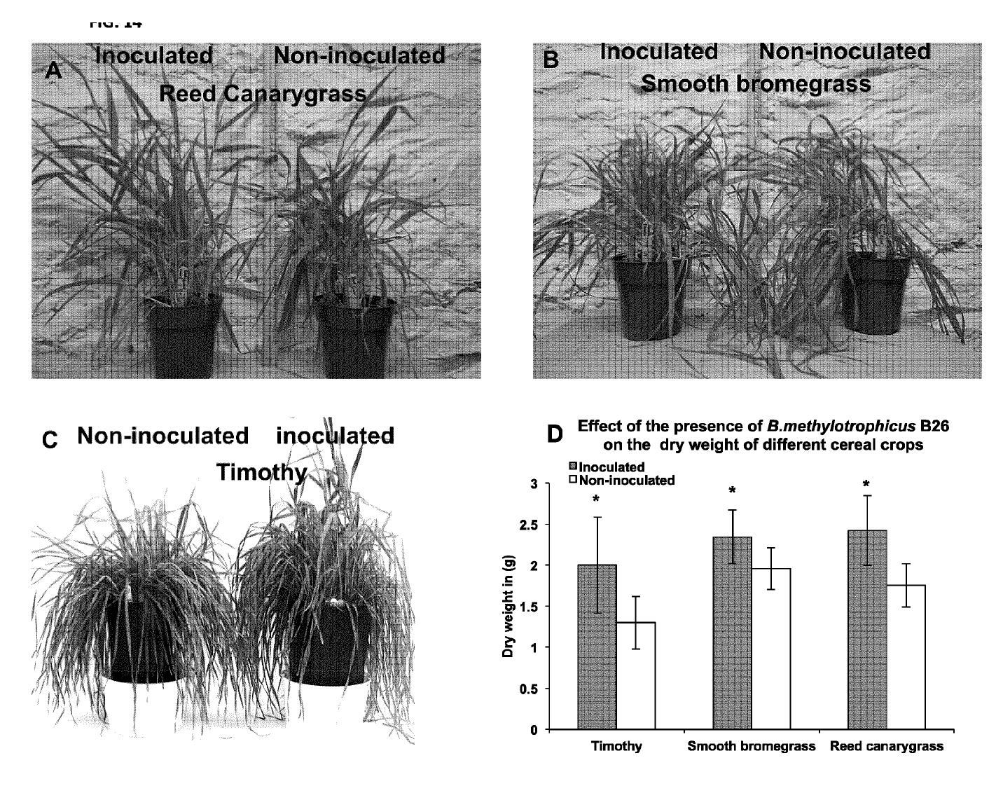

Similar differences were determined in the comparison of inoculated and non-inoculated grasses, such as reed canarygrass (FIG. 14A), smooth bromegrass (FIG. 14B), and timothy grass (FIG. 14C). Again the differences were assessed visually at harvest and via determination of their respective dry mass (FIG. 14D).

### Example 14: Formulation of Bacillus methylotrophicus B26 in Microspheres for Promoting Growth in Crop Plants

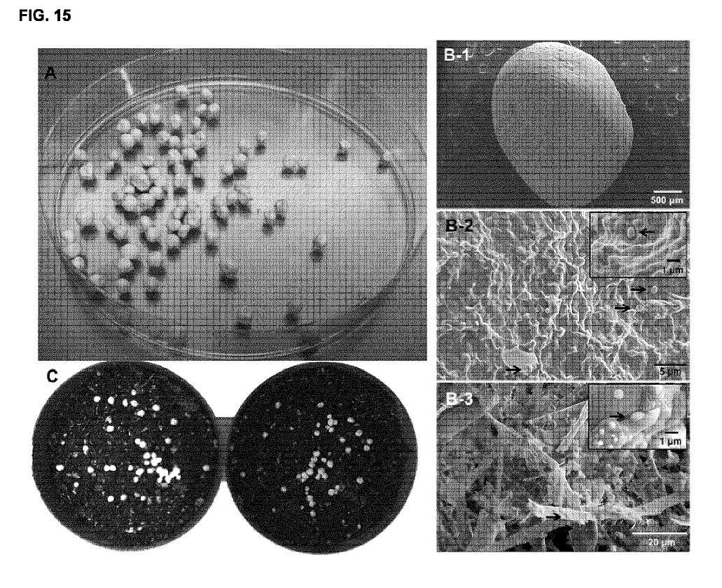

Production of microencapsulated Bacillus methylotrophicus B26. Pea protein isolate-alginate microspheres were prepared via extrusion technology according to (Khan, Korber et al. (2013)). The bacterial suspension was added to the polymer at a bacteria-to-polymer ratio of 1:10 (v/v). The bacteria loaded microspheres were formed via extrusion of the bacteria-polymer solution through a 26 G needle into a 0.05M CaCl₂ solution. The resulting microspheres were allowed to harden before they were collected and rinsed with sterilized water. Finally the microspheres were flash-frozen with liquid nitrogen and stored. See FIG. 15.

Survival of B. methylotrophicus 826 after freeze drying. In order to evaluate the survival of B. methylotrophicus after freeze drying, freeze-dried microspheres (0.1 g) were suspended and incubated in 9.9 mL of sterile modified phosphate buffer (Yasbin, Wilson et al. 1975) (Ammonium sulphate 0.2%, Potassium phosphate dibasic trihydrate 1.83%, Monopotassium phosphate 0.6%, Trisodium citrate 0.1% and Magnesium sulfate heptahydrate 0.02%) for 1 hour shaking at 250 rpm at room temperature to completely dissolve the microspheres. The viable cells were counted by spreading dilutions of the dissolved microspheres solution. Three technical replicate (three plates) were used to estimate the amount of CFU for each of the four replicate performed in the experiment.

Storage of microsphere at different temperatures. This experiment was designed in order to investigate the shelf life of encapsulated bacterial cells under various storage conditions. The freeze-dried microspheres placed into 50 mL falcon tubes and covered with aluminum foil to prevent light. The tubes were stored under three conditions: first at room temperature at 22° C., second in a fridge at 4° C. and third in a freezer at −15° C. Samples of microspheres (0.1 g) were withdrawn every 7 days for the first 56 days and then after 112 days of storage. Four biological replicates were used for each temperature condition. The samples were dissolved, diluted and spread plated on LBA agar plates to count viable cells. Freeze-dried bacteria, non-microencapsulated B26 were used as control. The cell suspension (0.1 mL) was transferred into a 1.5 mL centrifuge tube. The tubes were centrifuged using a microcentrifuge at 8000 rpm for 10 min and the liquid phase was removed. The tubes were freeze-dried for 48 h and stored in the same three conditions as the microspheres. To test for the viable cells, modified phosphate buffer was added to re-hydrate the cell pellets and incubated while shaking for 1 h following the same conditions as the microspheres. The viability of freeze-dried bacterial cells was tested every two weeks for the first 56 days. Three biological replicate were performed.

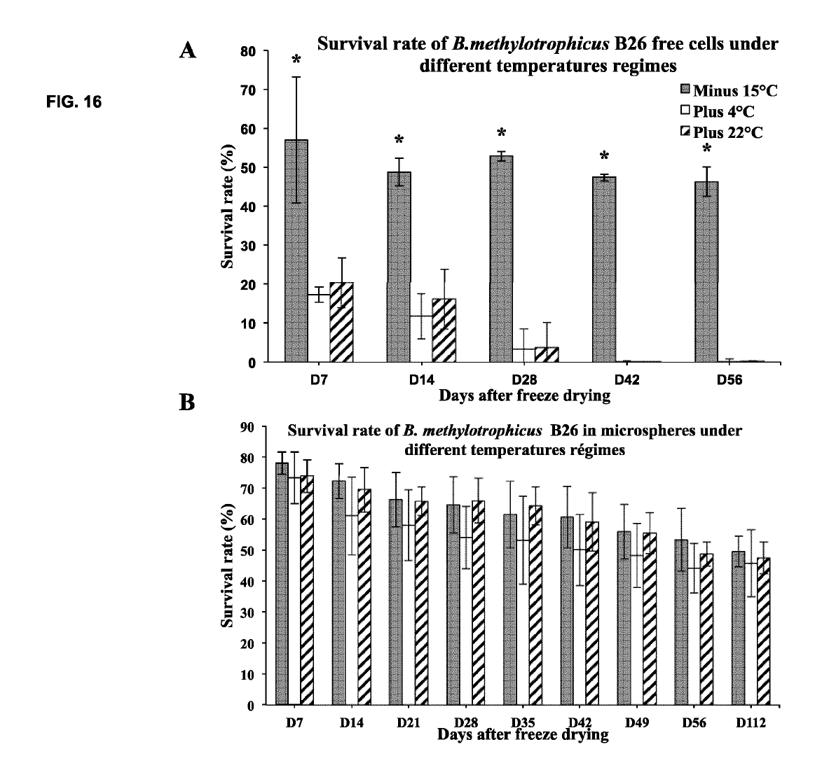

As shown in FIG. 16A the survival rate of free B. methylotrophicus B26 was stable at 15 C over 56 days, while cooler (4 C) and warmer conditions (22 C) led to the death of most bacteria after 28 days. In comparison, the survival rate of microsphere encapsulated B. methylotrophicus B26 bacteria dropped from 78% on day 7 after freeze dry treatment to 50% on day 112 after freeze dry treatment. While a storage temperature of 4 C seems to be less favorable, it does not seem to make a difference whether the microspheres are stored at 15 C or at 22 C (FIG. 16B).

### Example 15: Mode of Administration of Bacillus methylotrophicus B26 Microspheres

Re-inoculation and growth condition optimization. Brachypodium distachyon plants from the inbred line Bd21 (Brkljacic, Grotewold et al. 2011) and timothy (Phleum pretense) cultivar Novio seeds were surface sterilized according to Vain et al. (2008). Ten seeds were planted in each Pot (10×10 cm) containing sterilized Agro Mix® G6. Plants were stratified at 4° C. for 7 days after which they were placed in a climatically controlled chamber under a 16-h photoperiod with a light intensity of 150 μmoles/m²/s and a day/night temperature regime of 25/23° C. Plants were watered three times/week with sterile distilled water and fertilized every 2 weeks with N—P—K fertilizer 20-20-20/pot. Plants were thinned to five per pots after 21 days of growth and the experiment was kept for another 35 days. The experiment was repeated twice in different growth chamber.

Inoculation of plants with microspheres. Two different inoculation methods were evaluated for the use of B. methylotrophicus microspheres. In the first method called pre-planting or pre-inoculation treatment the microspheres were incorporated in the top 3 cm of the soil just before planting timothy and Brachypodium. In the second method called post-planting or post-inoculation treatment, microspheres were spread on the surface of the soil of already 21-day old non-inoculated timothy and Brachypodium plants. The amount of microspheres in both methods was adjusted to provide 5 million CFU per pot. Sterile microspheres devoid of bacteria were used as control.

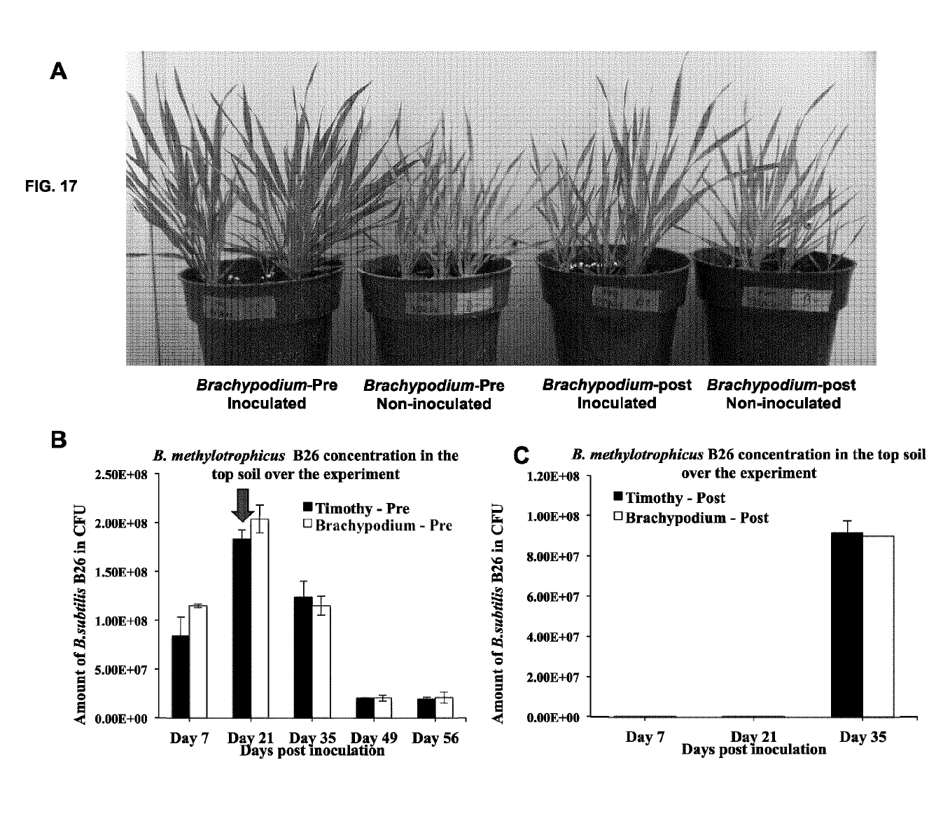

Microbiological and molecular monitoring of B. methylotrophicus 826. Soil from the top 3 cm were sampled from both experiments on days 7, 21, 35, 49 and 56 post planting for the pre-planting experiment and days 7, 21, 35 post inoculation for the post-planting experiment in order to evaluate the population abundance of B26 in the soil (FIG. 17). Post-planting treatment means the treatment where a seed first grows to a plant and is then inoculated contrary to the pre-planting treatment where the seed is inoculated at the time of sowing Special attention was made to separate the beads from the soil samples in order to obtain the actual abundance of Bacillus estimated as colony forming units (CFU)/gram of soil fresh weight via serial dilution method and plating on LBA. Four biological replications/plant species/inoculation methods were performed each time.

FIG. 17A shows the bacterized (inoculated) and non-bacterized (non-inoculated) Brachypodium plants obtained with the pre-inoculation or pre-planting treatment and with a post-inoculation or post-planting treatment. FIG. 17B shows the concentration of Bacillus methylotrophicus B26 in top soil over the period of 56 days when Bacillus methylotrophicus B26 loaded microspheres are applied to topsoil at the time of seeding Brachypodium or timothy, i.e. according to the pre-inoculation or pre-planting treatment mode. FIG. 17 C shows the concentration of Bacillus methylotrophicus B26 in top soil over the period of 35 days when Bacillus methylotrophicus B26 loaded microspheres are applied to topsoil when Brachypodium or timothy plants have reached an age of 21 days according to the post-inoculation or post-planting treatment mode. Thus the pre-inoculation method was the preferred method.

### Example 16: Material and Methods—Phenotypic and Metabolic Responses of Timothy Grass Bacterized with Bacillus methylotrophicus B26 to Drought Stress

Maintenance and preparation of Bacillus methylotrophicus 826 inoculum. The Bacillus methylotrophicus strain B26, previously isolated from switchgrass and fully characterized (Gagne-Bourque, Aliferis et al. 2013) was maintained as described supra.

Plant material and growth conditions. A pot experiment was conducted in growth chambers between Jul. 23 and Oct. 29, 2014 at the Agriculture and Agri-Food Canada Research Centre in Québec, QC, Canada in order to compare the effectiveness of B. methylotrophicus B26 for promoting growth and yield of timothy grass (Phleum pratense) under drought stress conditions. Seeds (cv Novio) were planted individually in microcell tray (1.5×1.5×3 cm) (The Blackmore Company, MI, USA) containing a soil mixture (10:1:1) of commercial topsoil: Perlite (Holiday perlite; V. I. L Vermiculite Inc., Lachine, QC, Canada): peat moss (Pro-mix BX; Premier Peat Moss, Rivière-du-Loup, QC, Canada). The soil mixture was autoclaved for 3 h at 121° C. for three constitutive days prior to planting. The experiment was conducted in growth chambers (Conviron, Model PGR15, Controlled Environments Limited, Winnipeg, Canada) for 6 weeks under a 16 h photoperiod with a day/night temperature regime of 20/10° C. Seedlings were watered as needed.

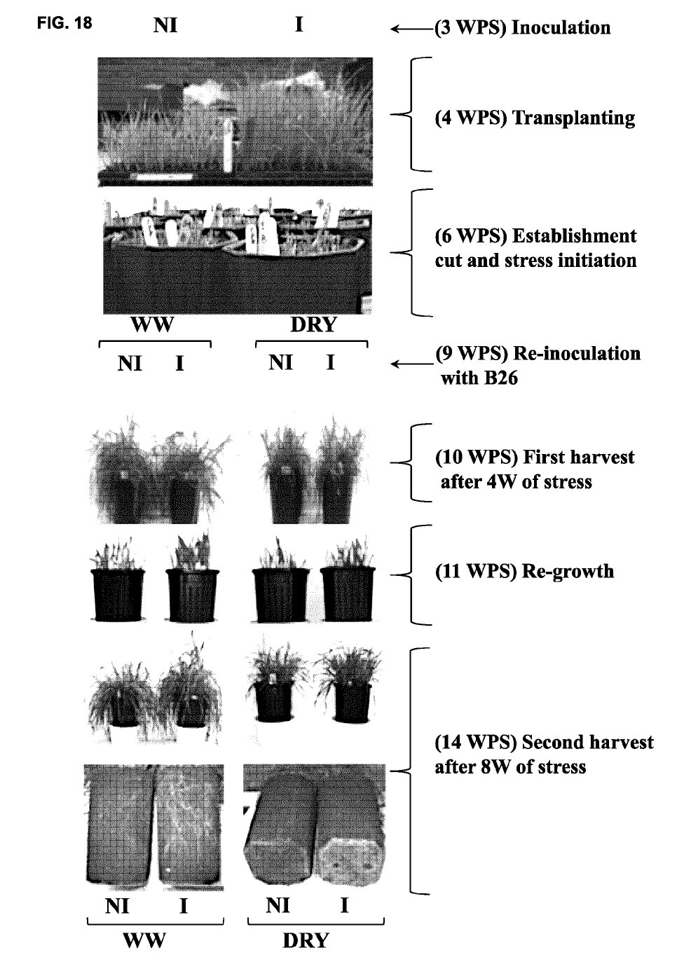

At three weeks post-seeding, each seedling was inoculated by pipetting 1 ml of phosphate buffer containing 106 CFU of B. methylotrophicus in the soil surrounding each plant in the tray (FIG. 18). Non-inoculated seedlings (Control) received 1 ml of sterile phosphate buffer. Re-inoculation of plants with strain B26 was performed at 9 weeks post-seeding following the same procedure as previously described.

At four weeks post-seeding Plants (10 per pot) were transplanted in pots of 30 cm wide by 32 cm deep (TPOT3, Stuewe and Sons, OR, USA) containing 4 kg of the same soil mixture as previously described. Inoculated and non-inoculated plants were incubated in 4 separate growth chambers. Pots were rotated and randomized between the four chambers allocated for each treatment every week until the end of the experiment in order to avoid confounding treatment effects with a chamber effect. Following a 2-week establishment period (i.e. 6 weeks post-seeding), plants were cut at a 3-cm height (establishment cut). Pots were returned to growth chambers, incubated at day/night temperatures of 25/15° C. and stress treatment was initiated. Well-watered (WW) and water stressed (DRY) plants were created as follows: (i) inoculated and well-watered (ii) non-inoculated plants and well-watered; (iii) inoculated and water stressed and (iv) non-inoculated and water stressed. Well-watered plants received water to field capacity 3 times per weeks based on pot weight. Water stressed treatments were enforced by reducing the water to ¼ of the amount that well-watered plants received. All pots received 100 ml of a solution of 1 g/liter of N—P—K fertilizer 20-20-20 (Plantprod, Laval, Qc, Canada) once a week.

A first harvest (H1) was performed on half of the plants of all treatments after 4 weeks of withholding water (i.e., 10 weeks post-seeding) when approximately 80% of the plants reached early anthesis stage (Simon and Park 1983). The remaining half was cut at 3 cm-height and left to regrow for an additional 4 weeks (i.e., 14 weeks post seeding) under the same conditions at which time a second harvest (H2; 8 weeks of withholding water) was performed in order to simulate the sequential harvests that are standard management practices for timothy in the field (FIG. 18). During each harvest, destructive measurements were taken from 8 pots (80 plants) for each growth and watering stress levels combination. Biomass of root and shoot, stage of development, photosynthesis and stomatal conductance, carbohydrates and amino acids analyses were conducted on the same 4 pots. While soil moisture, water content of plants and microbiological and molecular tests were performed on the remaining 4 pots. Therefore data were collected from a total of 64 pots.

Forage biomass and development stage During each harvest, the above ground biomass of plants in each pot was cut and the remaining roots and stubble were thoroughly washed to remove all traces of soil. Forage and root biomass were dried at 55° C. for 72 h, weighed and ground to pass a 1-mm screen with a Wiley mill (model 3379-k35, Variable Speed Digital ED-5 Wiley Mill, Thomas Scientific, Swedesboro, N.J.). Powdered samples were stored in 90 ml screw cap containers (Thermo Fisher Scientific, Ottawa, On, Canada) at room temperature for carbohydrates and amino acids analyses. Four biological replicates, each composed of 10-pooled plants were used.

Photosynthesis and conductivity measurement, and Leaf water potential and soil moisture. The photosynthetic rate and stomatal conductance were measured on the youngest fully developed leaf of a representative tiller from each pot using the LI-6400XT portable photosynthesis system (LI-COR, Lincoln, Nebr., USA). A function was generated to calculate boundary layer conductance for this chamber depending on leaf area and flow rate. Photosynthesis (μmol CO₂ m2-1s-1) and stomatal conductance (mol H₂O m2-1s-1) were determined according to the instrument's own formulae.

Leaf water potential and soil moisture Two representative non-flowering tillers per pot were selected and cut below the fourth youngest mature leaf. The leaf water potential was estimated using the portable pressure chambers 3005F01 Plant Water Status Console (Soil Moisture Equipment Corp., Santa Barbara, Ca, USA). Soil moisture percentage of each harvested pot was measured using reflectometry sensor technology (FieldScout TDR 100 equipped with the 20 cm rods, Spectrum Technologies Inc., Plainfield, Ill., USA). A degree of co-regulation exists between stomatal movements which is linked to Leaf conductance (Jarvis 1976) and photosynthetic rates (Reddy, Chaitanya et al. 2004).

Detection, enumeration and quantification of B. subtilis 826. To ensure that B26 successfully and systemically colonized different plant tissues of timothy and that intracellular spread of B26 was sustained in the respective tissues, bacteria cell numbers and DNA copy number were determined in root and shoot tissues and in rhizosphere soil of inoculated and non-inoculated plants subjected or not to water stress. At each harvest, four plants/pot of each replicate of all treatments were randomly selected and shoots and roots were separated and pooled. Roots were gently shaken to collect rhizosphere soil. Collected tissues and soil samples were rapidly processed for B. subtilis abundance numbers using culture-dependent (CFU counts) and culture-independent methods (DNA copies). Irrespective of the method applied, all collected tissue samples were surface sterilized following a stepwise protocol of ethanol, sodium hypochlorite and water as previously described (Gagne-Bourque, Aliferis et al. 2013).

Homogenized tissue samples (200 mg) and rhizospheric soil (1 g) from WW and DRY treatments inoculated or not were serially diluted in phosphate buffer and plated on LBA (Skinner, Jones et al. 1952). Prior to dilution, rhizospheric soil was suspended in 9 mL of phosphate buffer, shaken for 30 min and incubated at 95° C. for 5 mins. Plates were incubated at 37° C. for 24 h. Colony forming units (CFUs) were determined and calculated to Log CFU per gram of fresh weight of tissue or soil. There were four biological replicates each consisted of four plants for each treatment. Root tissues of Harvest 2 were lignified and impossible to properly homogenize, and thus were not subjected to bacterial enumeration. The presence of B. subtilis B26 cells inside inoculated plants subjected or not to water stress was also confirmed by quantitative real-time PCR (QPCR) assays. Surface sterilized and freeze-dried plant tissues were reduced to powder in liquid nitrogen, and genomic DNA was extracted from 200 mg of powdered tissue using the CTAB method (Porebski, Bailey et al. 1997). Genomic DNA from B. subtilis B26 colonies was extracted by direct colony PCR (Woodman 2008). Briefly, single colonies were mixed with sterile distilled water, incubated at 95° C. followed by centrifugation and the supernatant was used as template DNA in conventional PCR assays. B. subtilis B26 amplicons from strain specific primers (Gagne-Bourque, Aliferis et al. 2013) were purified, cloned and used to build a standard curve for QPCR assays following (Gagné-Bourque, Mayer et al. 2015).

Carbohydrate and Amino Acid extraction Accumulation of solutes such as carbohydrates, amino acids as drought protection indicators is well known in grasses under drought stress (Spollen and Nelson 1994; Hanson and Smeekens 2009; Krasensky and Jonak 2012). At each harvest, 200 mg of dried ground material was incubated in 7 mL of deionised H₂O at 80° C. for 20 min. Tubes were then incubated overnight at 4° C. and were subsequently centrifuged 10 min at 1500×g. A 1-mL sub-sample of the supernatant was collected for quantification of soluble carbohydrates. All extracts were stored at −80° C. until analysis could be completed.

Soluble sugars and low degree of polymerization fructans. The soluble sugars sucrose, glucose, fructose, raffinose and low degree of polymerization (LDP) fructans (degree of polymerization [DP] 3 to DP9) were analyzed using a Waters ACQUITY Ultra Performance Liquid Chromatography (UPLC) analytical system controlled by the Empower II software (Waters, Milford, Mass., USA), and following the procedure of Piva et al. (2013) for conditions of elution and eluent collections. Peak identity and quantity of sucrose, glucose and fructose were determined by comparison to standards. The degree of polymerization of LDP fructans was established by comparison with elution time of purified standards from Jerusalem artichoke (Helianthus tuberosus L.) and the quantity was determined by reference to a fructose standard.

High degree of polymerization fructans High degree of polymerisation fructans (HDP), from DP 10 to DP 200 were analyzed using a Waters HPLC analytical system controlled by the Empower™ II software. Samples were centrifuged for 3 minutes at 16,000 g and kept at 4° C. throughout the analysis within the Waters 717 plus autosampler. HDP fructans were separated on a Shodex™ KS-804 column preceded by a Shodex™ KS-G precolumn (Shodex, Tokyo, Japan) eluted isocratically at 50° C. with deionized water at a flow rate of 1.0 mL min-1 and were detected on a Waters™ 2410 refractive index detector. The degree of polymerization of HDP fructans was estimated by reference to a standard curve established with seven polymaltotriose pullulan standards (Shodex Standard P-82) ranging from 0.58×10⁴ to 85.3×10⁴ of molecular weight. The concentration of both LDP and HDP fructans is expressed on an equivalent fructose basis.

Total Starch. Total starch was extracted with methanol from the non-soluble residues left after water extraction and quantified following a gelatinization and enzymatic digestion with amyloglucosidase steps (Blakeney and Mutton 1980). Starch was quantified as glucose equivalents following enzymatic digestion with amyloglucosidase (Sigma™ A7255; Sigma-Aldrich Co., St. Louis, Mo.) and colorimetric detection with hydrobenzoic acid hydrazide method of (Blakeney and Mutton 1980).

Amino acid Analysis Twenty-one amino acids were separated and quantified using Waters ACQUITY™ UPLC analytical system controlled by the Empower™ II software (WATERS, Milford, Mass., USA). The amino acids were derivatized using AccQ Tag Ultra Reagent™ (6-aminoquinolyl-N-hydroxysuccinimidyl carbamate). The derivatives were separated on an AccQ Tag Ultra column (2.1×100 mm) and detected with Waters ACQUITY™ Tunable UV detector at 260 nm under the chromatographic conditions described in Cohen (2000). Peak identity and amino acid quantity were determined by comparison to a standard mix containing the 21 amino acids. Results from amino acid determination were expressed as concentrations on dry weight basis (μmol g-1 DW). Cohen, S. A. 2000. Amino acid analysis using precolumn derivatization with 6-aminoquinolyl-N-hydroxysuccinimidyl carbamate.

Statistical analysis Timothy plants (10 plants per pot) were subjected to two watering levels. For each level, plants were inoculated or not with B. subtilis B26 and were harvested at two time points. At each harvesting date, 4 pots were processed for phenotypic and biochemical measurements and 4 other pots were processed for plant water content, microbiological and molecular measurements. Pots were put in a complete randomized design.

One-way ANOVA was performed using the JMP 10.0 software (SAS Institute, Cary, N.C., USA on phenotypic measurements (i.e., biomass, photosynthesis rate, stomatal conductance, water potential, and soil moisture), and on microbial abundance (CFU numbers and DNA copies). All experimental data were tested for statistical significance using Tukey HSD with a magnitude of the F-value (P=0.05). Each harvest was analysed separately.

Multivariate analysis was performed on the carbohydrate and amino acids contents. Data were combined into a data matrix that was subjected to multivariate analyses using the SIMCA-P+v.12.0 software (Umetrics, MKS Instruments Inc.) as previously described (Aliferis, Faubert et al. 2014). For the preliminary evaluation of data, principal component analysis (PCA) was performed. The detection of biomarkers was based on orthogonal partial least squares-discriminant analysis (OPLS-DA) regression coefficients (P<0.05) and standard errors were calculated using Jack-knifing with 95% confidence interval. The performance of the models was assessed by the cumulative fraction of the total variation of the X's that could be predicted by the extracted components [Q2 (cum)] and the fraction of the sum of squares of all X's (R2 X) and Y's (R2 Y) explained by the current component.

### Example 17: Phenotypic and Metabolic Responses of Timothy Grass Bacterized with Bacillus methylotrophicus B26 to Drought Stress

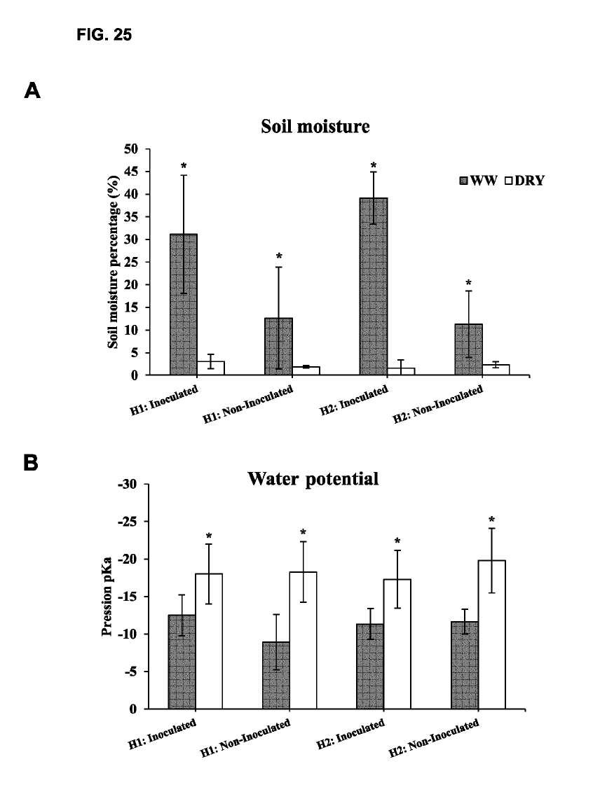

The successful establishment of the water stress was paramount to the success of this experiment. Soil moisture content (FIG. 25 A) and water potential (FIG. 25. B) were measured at both harvest time points in order to ensure that a significant difference was established in-between the treatments. At both harvests a significant and constant difference in water concentration between the two watering levels was observed.

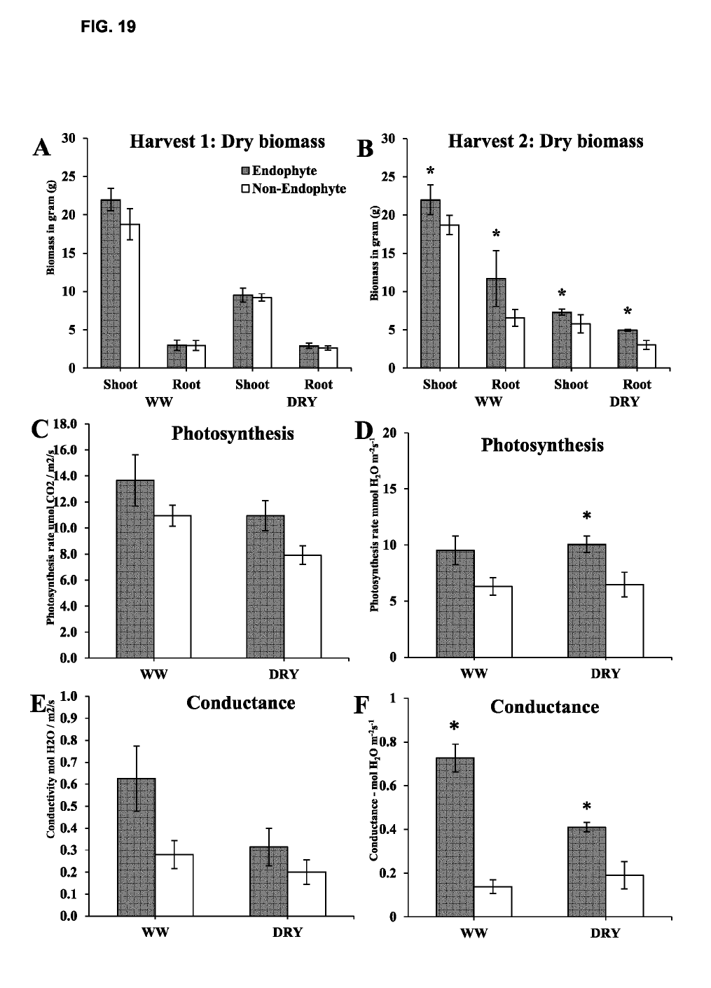

Inoculation with endophytic B. methylotrophicus strain B26 significantly promoted both root and shoot growth under both well-watered (WW) and water stressed (DRY) conditions only at H2 time point (FIGS. 19A, 19B). Maximum response, up to 26.6% and 63.8% in shoot and root dry mass, respectively compared to the control was recorded under water stress conditions (FIG. 19B). Growth stimulation of timothy is most likely related to P solubilization and the production of indole-3-acetic acid (IAA) and the cytokinin zeatin riboside by strain B26 as we previously reported (Gagne-Bourque, Aliferis et al. 2013).

Plants inoculated with Bacillus methylotrophicus B26 resulted in higher photosynthetic rate by 55.2% and also in stomatal conductance by 214.9% under water stress conditions compared to the controls at H2 only (FIGS. 19C, D, E and F) leading to better survival, and greater root and shoot biomass compared to the non-inoculated plants grown under the same condition (FIGS. 19 A and B).

### Example 18: Successful and Stable Colonization of Timothy by B. methylotrophicus Strain B26

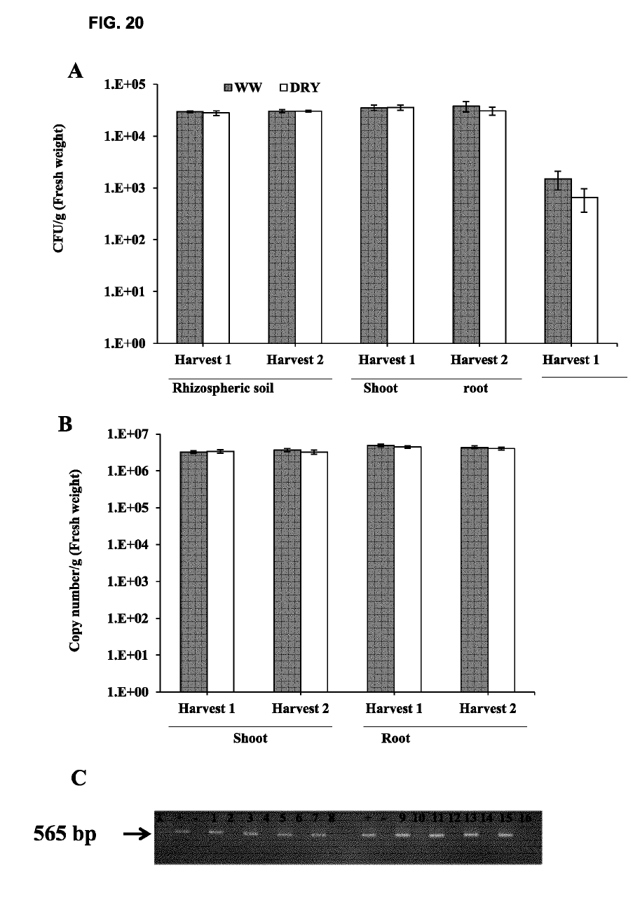

B. methylotrophicus B26 successfully colonized the forage grass timothy and influenced its growth under normal and water stress. Strain B26 efficiently colonized the rhizosphere and timothy roots and was also intimately associated with the plant since it could be isolated from the interior of root and shoot tissues of surface sterilized inoculated plants at both harvest points (FIG. 20). The success of internal and systemic colonization of timothy by B26 was confirmed by culture-dependent (FIG. 20A) and independent methods (FIG. 20B). Re-isolation and quantification of strain B26 by the plating method in different surface-sterilized tissues of well-watered (WW) and drought stressed (DRY) plants clearly demonstrate that B. methylotrophicus B26 can form sustaining and endophytic populations in roots, shoots as well as in the soil around the roots of timothy (FIG. 20). The presence of B. methylotrophicus B26 in different tissues of timothy was confirmed by QPCR in inoculated plants (FIG. 20B). An amplicon with the expected product size of 565 bp was successfully amplified using species-specific primers for B. methylotrophicus B26 from DNA extracted from each tissue type (FIG. 20C).

Population numbers of B26 in soil and timothy shoot and root tissues were similar ranging from log₁₀ 4.44 to 4.57 log₁₀ CFU at both harvests and so are the absolute DNA copy numbers which were sustained in the roots and shoots. These densities are comparable to what had been reported for Bacillus species including B. subtilis (van Elsas, Dijkstra et al. 1986; Rai, Dash et al. 2007; Ji, Lu et al. 2008; Liu, Qiao et al. 2009).

### Example 19: Robustness of the Model

To address the question regarding the comparison of individual amino acids and sugars of bacterized plants expressed to stress or not required the application of Principal component analysis (PCA).

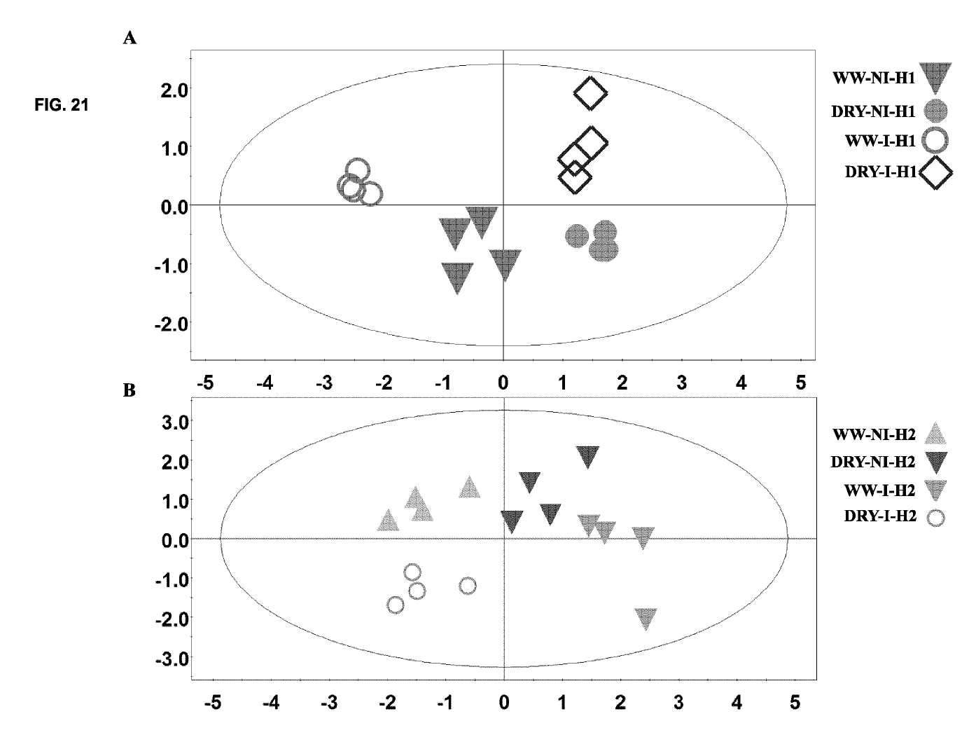

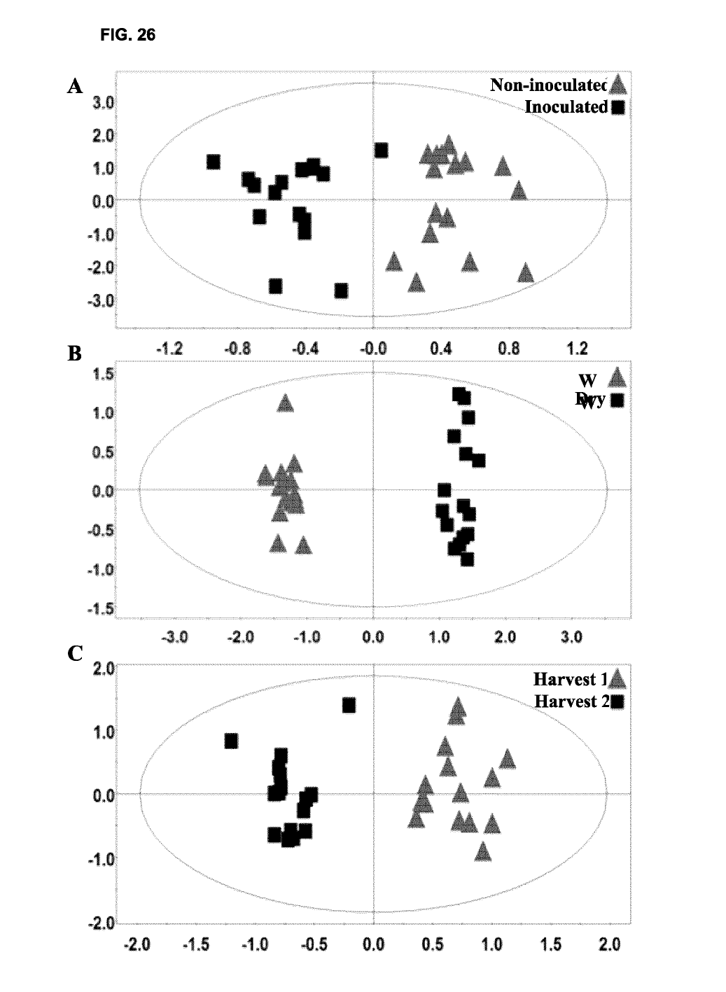

Principal component analysis (PCA) was performed initially for the whole dataset revealing no outliers (data not shown). In a second step, orthogonal projections to latent structures-discriminant analysis (OPLS-DA) with a regression coefficients (P<0.05) was used. OPLS-DA revealed a strong discrimination between inoculated and non-inoculated plant (FIG. 26A) between the watering level (FIG. 26B) and between the two harvests (FIG. 26C). Furthermore, the tight clustering among biological replications confirms the robustness and reproducibility of the experimental protocol (FIGS. 21A and B).

### Example 20: Determination of Carbohydrate Metabolism in Bacterized Timothy

The inventors sought to determine whether the increased drought tolerance of timothy bacterized with B. methylotrophicus B26 is manifested in accumulation of key water-stress induced metabolites (Chen and Jiang 2010; Krasensky and Jonak 2012). The inventors assessed the differences in metabolite accumulation in shoots and roots in inoculated or non-inoculated timothy plants over an extended 8-week period of water deficit stress. Most experiments of this nature, to the best of the inventors' knowledge, are performed on young plants with treatments of withholding water not exceeding beyond 1 week (Timmusk and Wagner 1999; Sandhya, Ali et al. 2010; Arzanesh, Alikhani et al. 2011; Vardharajula, Zulfikar Ali et al. 2011).

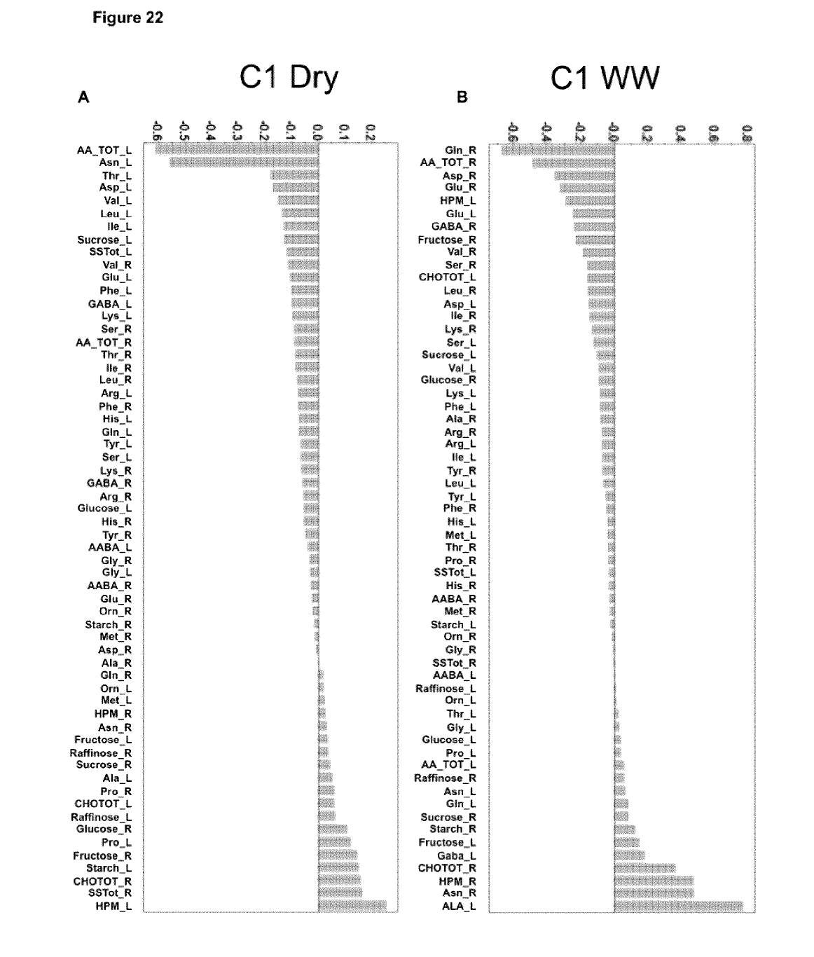

In the present experiment, bacterized plants accumulated more total carbohydrates and total soluble sugars in shoots compared to roots of non-stressed and stressed plants (FIGS. 22, 23 and 24A). Inoculation of timothy with strain B26 improved most notably sucrose and fructan (labeled as HPM_L or HPM_R) contents of leaves under non-stressed and drought stressed conditions over a period of 8 weeks of withholding water, while glucose increased in plants leaves after 4 weeks and in root after 8 weeks of withholding water (FIGS. 22, 23 and 24). Such increases are directly linked to the presence of strain B26 and strongly indicate that B. methylotrophicus helps increasing biosynthesis of sugars that allow for better osmotic adjustment thus alleviate stress effect.

Drought stress frequently enhances allocation of dry matter and preferential accumulation of starch and dry matter in roots of some plants (De Souza and Da Silva 1987; Leport, Turner et al. 1999) as adaptation to drought, which can enhance water uptake (Farooq, Wahid et al. 2009). The prolific and extensive root system and dry mass (FIGS. 18 and 19) of inoculated plants ensured a sufficient water supply under drought conditions, however the presence of the endophyte did not generally improve total carbohydrates and contents of some soluble sugars but the osmotically active molecule, sucrose, was increased by 1.33 fold in inoculated roots after 8 weeks of withholding water.

### Example 21: Determination of Amino Acids Metabolism in Bacterized Timothy

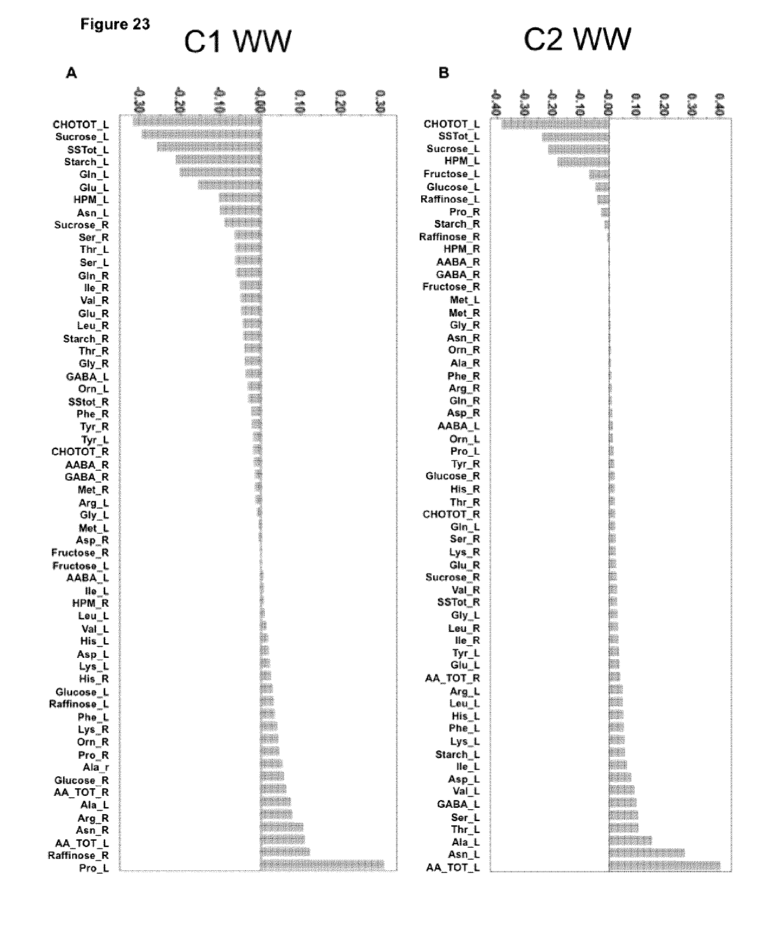

A total of 21 amino acids were measured in shoots and roots of bacterized watered and stressed timothy plants. Many amino acids that are members of the aromatic, pyruvate, glutamate and aspartate families were produced in greater quantities in plants inoculated with B. methylotrophicus B26 under water-stress conditions (FIGS. 22-24). The majority of amino acids increased in shoots and roots of bacterized plants exposed or not to 4 week-period of water deficit (FIGS. 22 A and B), however the effect of inoculation on amino acid content was more pronounced in leaves under drought stress (FIGS. 22-23).

### Example 22: Determination of Aromatic Amino Acids Metabolism in Bacterized Timothy

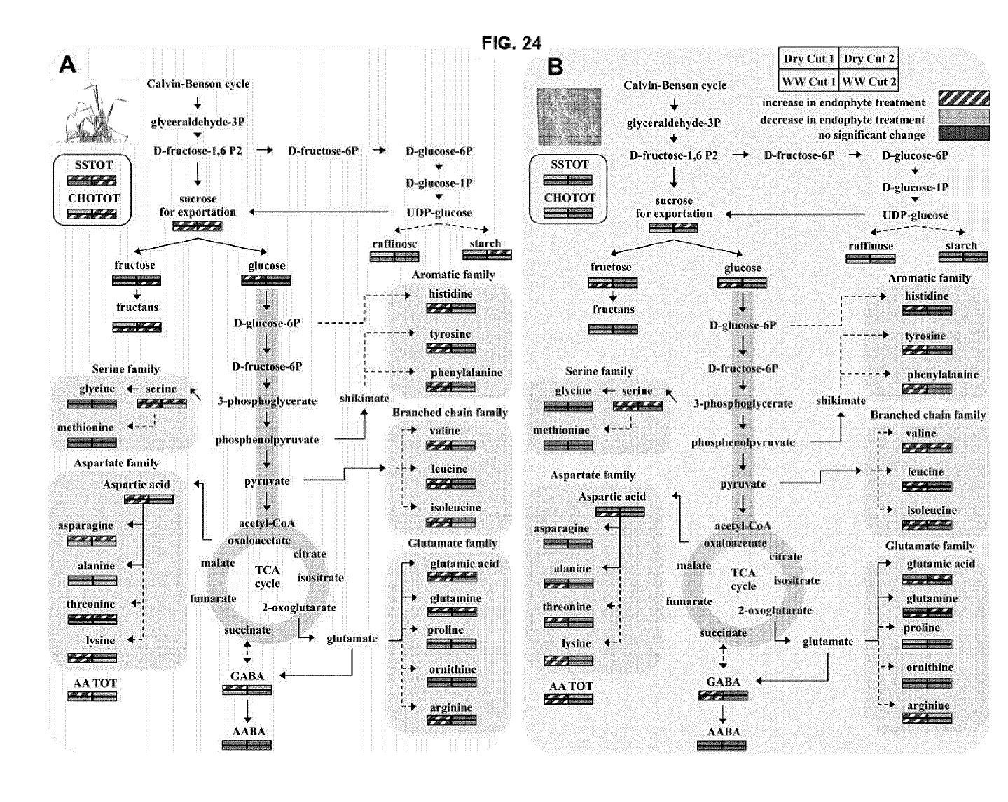

The increased levels of histidine, tyrosine and phenylalanine were highly consistent in bacterized timothy plants that were exposed or not to 4 week-period of water deficit. (FIGS. 22 and 24).

Levels of these aromatic amino acids have been implicated in drought stress in maize and wheat (Harrigan, Stork et al. 2007; Witt, Galicia et al. 2011; Bowne, Erwin et al. 2012). Histidine, an essential amino acid required for plant growth and development, functions as a metal-binding ligand and as a major part of metal hyperaccumulator molecule leading to alleviation of heavy metal stress (Sharma and Dietz 2006), but also is reported to be play a role in abiotic stress (Harrigan, Stork et al. 2007). Tyrosine and phenylalanine are synthesized through the shikimate pathway and serve as precursors for a wide range of secondary metabolites, some of which are ROS scavengers (Less and Galili 2008; Gill and Tuteja 2010). Water deficit enhances the production of reactive oxygen molecules and the maintenance or increase in the activity of enzymes involved in removing toxic ROS to avoid cellular damage is regarded as an important factor in tolerance to dehydration (Chaves, Maroco et al. 2003). Both amino acids may serve as buffer antioxidants and as ROS scavengers (Gill and Tuteja 2010).

### Example 23: Determination of Branched Chain Family Amino Acids Metabolism in Bacterized Timothy

Valine, leucine and isoleucine, the branched amino acids increased in leaves and roots of bacterized timothy plants (FIGS. 22-24), however, their accumulation was most prominent in leaves of bacterized plants exposed to a 4-week period of stress (FIGS. 22 and 24) and in roots of bacterized plants exposed to an 8-week period of stress (FIG. 23).

These results support what has been previously reported in wheat and pea that branched amino acids play an active role in plant tolerance or avoidance mechanism to drought (Charlton, Donarski et al. 2008; Bowne, Erwin et al. 2012). Taylor and co-workers (2004. #866) stated that branched amino acids may provide a source of energy in sugar starved Arabidopsis, while Joshi and Jander 2009 #687) working also on Arabidopsis proposed that they can act as osmolytes thus increasing plant drought tolerance.

### Example 24: Determination of Aspartate Family Amino Acids Metabolism in Bacterized Timothy

Most notably was the considerable accumulation of asparagine in leaves of bacterized plants exposed to an extended 8 week-period of stress (FIGS. 23 and 24). Concomitant with asparagine, threonine accumulation in the same tissue was also observed. On the contrary, B. methylotrophicus improved threonine levels in roots of plants exposed to 4 weeks of stress only. Both tissues of bacterized plants that were exposed to hydric stress for 4 weeks and those that were well watered accumulated lysine. The levels of alanine, classified in aspartate family by Aliferis et al. (2014), decreased due to endophyte or water deficit stress in leaves (FIGS. 22-24). Taken together, there is no consistent endophyte effect on these amino acids levels between shoots and roots.

A similar trend was reported for water stressed tall fescue infected with the fungal endophyte Neotyphodium coenophialum (Nagabhyru, Dinkins et al. 2013). Aspartic acid, asparagine, threonine and lysine have been reported to accumulate in a range of plant tissues under stress (Barnett and Naylor 1966; Venekamp 1989; Kusaka, Ohta et al. 2005; Lea, Sodek et al. 2007).

### Example 25: Determination of Glutamate Family Amino Acids Metabolism in Bacterized Timothy

B. methylotrophicus B26 improved the content of glutamic acid and glutamine but not proline in plants that are water-stressed or not for an extended period of stress, while arginine increased in roots and shoots of inoculated plants exposed or not to 4 weeks of stress (FIGS. 22-24). As expected and in agreement with the literature, proline level in leaves and roots of non-inoculated plants substantially increased owing to water stress (Verslues and Sharma 2010), however inoculation with B. methylotrophicus did not improve proline concentration in the leaves and roots of non-stressed plants (FIGS. 22-24). This indicates that proline biosynthesis is not a mechanism used by B. methylotrophicus B26 to confer a greater drought resistance to timothy but the biosynthesis of proline precursors is.

Proline is one of the known markers of water and salt stress in plants. It is a natural osmoproctectant and is a major stress-signalling molecule (Chaves, Maroco et al. 2003; Krasensky and Jonak 2012). Proline accumulation in plants is usually coupled with increases in its precursor glutamic acid, ornithine and arginine (Ashraf and Foolad 2007).

### Example 26: Determination of Serine Amino Acid Metabolism in Bacterized Timothy

Inoculation of plants with B26 improved serine content under stressed and well-watered conditions, however, well-water inoculated plants accumulated more serine in both leaves and roots by 1.35 and 1.29 fold, respectively. Despite the increase of serine, one would expect that glycine content would have changed. Interestingly, levels of glycine in leaves and roots of inoculated non-stressed and stressed plants remained the same (FIGS. 22-24) indicating that the bacterium had no bearing on serine levels.

Serine is a precursor of the organic osmolyte glycine betaine, which accumulates in a variety of plant species in response to environmental stresses such as drought, salinity, extreme temperatures, UV radiation and heavy metals. (Ashraf and Foolad 2007). Studies on drought-stressed Bermuda grass and pearl millet also showed that glycine content in different plant tissues was not affected by drought (Barnett and Naylor 1966; Kusaka, Ohta et al. 2005).

### Example 27: Determination of γ-Aminobutyric Acid (GABA) Metabolism in Bacterized Timothy

The accumulation of GABA in shoots exposed to stress and roots of stressed and not stressed plants were improved by the presence of the endophyte (FIGS. 22-24). Levels of α-Aminobutyric acid (AABA) an isomer form of the bioactive β-aminobutyric acid (BABA) also involved in drought protection were unchanged. Similarly, pre-treatment of Arabidopsis with AABA failed to induce drought tolerance (Jakab, Ton et al. 2005).

The non-protein γ-aminobutyric acid GABA functions as an osmolyte and mitigates water stress (Kinnersley and Turano 2000), thus its levels would be expected to be greatest in tissues exposed to stress.

### Example 28: Determination of Contribution to Osmolytes Pool from the Internal Production of B. methylotrophicus B26

Plant associated bacteria may also exude osmolytes in response to stress, which may act synergistically with plant-produced osmolytes and stimulate growth under stressed conditions (Madkour, Smith et al. 1990; Paul and Nair 2008).

The osmolytes of B. methylotrophicus bacterized plants in response to stress are determined. The increase in certain osmolytes in inoculated stressed timothy plants can be, in part, created by B. methylotrophicus B26.

### Example 28: ACC Deaminase Production

The ability of plant growth promoting bacteria to produce 1-aminocyclopropane-1-carboxylate (ACC) deaminase (Azevedo et al 2000) to lower plant ethylene levels is a well-known mode of action that helps the plant to increase its drought resistance (Glick 2012). The bacteria consume ACC, a precursor of ethylene, via ACC deaminase thus lowering plant ethylene production. Ethylene is produced by the plant following different types of biotic and abiotic stresses (Glick 2014). Following stress perception, it is believed that plants produce ethylene in two successive “events”. The first “event” triggers the initiation of transcription of genes that encode plant defensive and protective proteins (Glick 2014). The second ethylene production “event” is generally detrimental to plant growth and is often involved in initiating processes such as senescence, chlorosis and leaf abscission. Thus the high level of plant ethylene can increase the effects of the stress. It this therefore believed that lowering the amount of ethylene production in the second “event” should decrease the amount of damage to the plant that occurs as a consequence of the stress.

The presence of ACC deaminase in rhizobial bacteria has been so closely linked to the potential to confer drought resistance to plants that a person skilled in the art of identifying drought resistance conferring bacteria would enrich for said bacteria by subjecting a soil sample to an ACC deaminase selection process. To the inventors' knowledge, there has only been one report in cucumbers where a consortium of three strains (Bacillus cereus AR156, Bacillus subtilis SM21, and Serratia spec XY21) has led to the induction of drought tolerance without the presence of ACC deaminase in any of the three strains (Wang et al 2012). However, it is unclear whether one strain on its own could provide these characteristics.

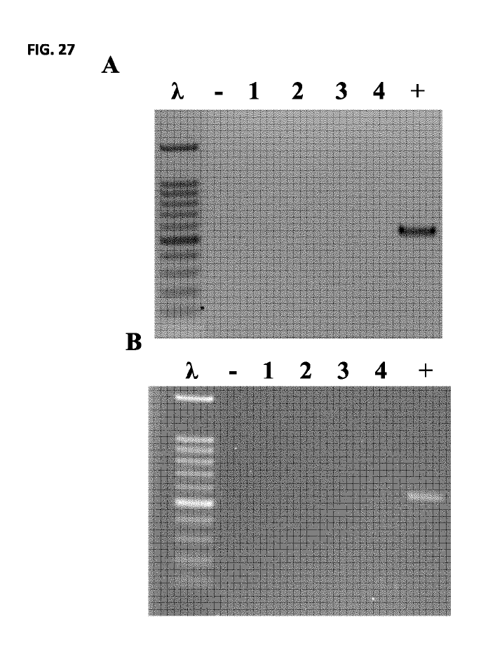

The inventor's tested the ability of B. methylotrophicus B26 to produce ACC deaminase both biochemically and genetically. A number of primers were designed from known ACC deaminase genes of Bacillus spp (specific primers) and from sequences of conserved regions designed from a mixture of bacteria (general primers). All the sequences used for the design of the primers are published on NCBI and summarized in Table 5. None of the primer pair sets led to the amplification of an ACC deaminase transcript in B26 suggesting that B26 does not express the ACC deaminase gene (FIG. 27).

TABLE 5
Primer sets used to amplify the ACC deaminase gene(s)

| Type | Primers | 5′ To 3′ |
|---|---|---|
| Specific | ACC1_Forward | CTGTTCCGAGTATCCCTATG (SEQ ID NO: 46) |
| | ACC1_Reverse | CGAGCAGATCACGATGTA (SEQ ID NO: 47) |
| Specific | ACC2_Forward | ACTACTCCGACACTGTATATG (SEQ ID NO: 48) |
| | ACC2_Reverse | CCAATGTCGAAACCTTCAG (SEQ ID NO: 49) |
| Specific | ACC3_Forward | CAGCAGGAAAAGGATTTGGG (SEQ ID NO: 50) |
| | ACC3_Reverse | ACTCCACTGAATTGAACCCG (SEQ ID NO: 51) |
| GENERAL | ACC_Gen_Forward | GCACAAGCACACACTTCATA (SEQ ID NO: 52) |
| | ACC_Gen_Reverse | AAGCGTGAAGACTGCAATAG (SEQ ID NO: 53) |

Three biochemical assays were used to assess the ability of B26 to use ACC as source of nitrogen. Bacterial growth on ACC as source of nitrogen indicates the ability of a bacterium to produce functional ACC deaminase. The three methods were described in detail in Penrose and Glick (2003).

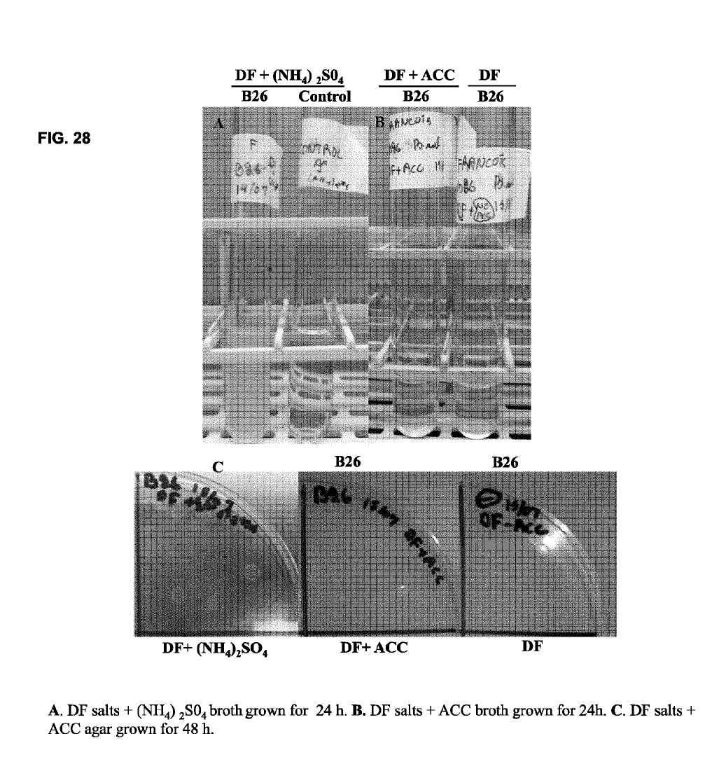

The first method consisted in growing the bacteria in liquid culture in a rich media (LB) for 24 hours at 37° C. at 200 RPM and transferring 0.1 ml of the culture in 5 ml of DF salt media (DF) (Dworking and Foster, 1958) containing 2.0 g (NH₄)₂SO₄ as nitrogen source (FIG. 28A). The bacteria were left to grow for 24 h under the same conditions as described above. The bacteria were pelleted and washed in DF salt without nitrogen. Finally the bacterial pellet was re-suspended in 5 ml of DF salt media containing 3 mM ACC as source of nitrogen. The bacteria were left to grow again for 24 hours under the same conditions. Bacillus methylotrophicus B26 was able to grow in the DF salt media containing (NH₄)₂SO₄ but unable to grow in the DF media with ACC as source of nitrogen, which showed its inability to produce ACC deaminase (FIG. 28B).

In the second method bacteria were transferred and grown on DF-agar supplemented with 30 mMol ACC per plate after enrichment in ((NH₄)₂SO₄ containing DF salt medium. The plates were incubated at 37° C. for 48 hours. No growth was detected confirming that Bacillus methylotrophicus B26 does not produce any ACC deaminase (FIG. 28C).

The third method consisted in quantifying of ACC deaminase activity by measuring the amount of a-ketobutyrate, the reaction product of ACC cleaved by ACC deaminase. The a-ketobutyrate concentration was measured as absorbance at 540 nm of a sample compared to a standard curve of the product ranging from 0.1 to 1 μM. This method again confirm Bacillus methylotrophicus B26's ACC deaminase deficiency.

### REFERENCES

Agbessi S, Beauséjour J, Déry C, Beaulieu C. (2003). “Antagonistic properties of two recombinant strains of Streptomyces melanosporofaciens obtained by intraspecific protoplast fusion”. Appl Microbiol Biotechnol. August; 62(2-3):233-8.
Aliferis, K. A., D. Faubert, et al. (2014). “A metabolic profiling strategy for the dissection of plant defense against fungal pathogens.” PLoS ONE 9(11): e111930.
Araus, J. L., G. A. Slaffer, et al. (2002). “Plant breeding and drought in C3 cereals: What should we breed for?” Annals of Botany 89(7): 925-940.
Arkhipova T N, Veselov S U, Melentiev A I, Martynenko E V, Kudoyarova G R (2005) Ability of bacterium Bacillus subtilis to produce cytokinins and to influence the growth and endogenous hormone content of lettuce plants. Plant and Soil 272: 201-209.
Arkhipova, T., E. Prinsen, et al. (2007). “Cytokinin producing bacteria enhance plant growth in drying soil.” Plant and Soil 292(1-2): 305-315.
Arzanesh, M., H. Alikhani, et al. (2011). “Wheat (Triticum aestivum L.) growth enhancement by Azospirillum sp. under drought stress.” World Journal of Microbiology and Biotechnology 27(2): 197-205.
Ashraf, M. and M. R. Foolad (2007). “Roles of glycine betaine and proline in improving plant abiotic stress resistance.” Environmental and Experimental Botany 59(2): 206-216.
Azevedo, J. L., W. Maccheroni Jr., et al. (2000). “Endophytic microorganisms: a review on insect control and recent advances on tropical plants.” Electronic Journal of Biotechnology 3: 15-16.
Barnett, N. M. and A. Naylor (1966). “Amino acid and protein metabolism in Bermuda grass during water stress.” Plant Physiology 41(7): 1222-1230.
Bélanger, G., Y. Castonguay, et al. (2006). “Winter damage to perennial forage crops in eastern Canada: Causes, mitigation, and prediction.” Canadian Journal of Plant Science 86(1): 33-47.
Blakeney, A. B. and L. L. Mutton (1980). “A simple colorimetric method for the determination of sugars in fruit and vegetables.” Journal of the Science of Food and Agriculture 31(9): 889-897.
Boiero, L., D. Perrig, et al. (2007). “Phytohormone production by three strains of Bradyrhizobium japonicum and possible physiological and technological implications.” Applied Microbiology and Biotechnology 74(4): 874-880.
Bowne, J. B., T. A. Erwin, et al. (2012). “Drought responses of leaf tissues from wheat cultivars of differing drought tolerance at the metabolite level.” Molecular Plant 5(2): 418-429.
Brkljacic J, Grotewold E, Scholl R, Mockler T, Garvin D F, et al. (2011) Brachypodium as a model for the grasses: today and the future. Plant Physiology 157: 3-13.
Calvo, P., L. Nelson, et al. (2014). “Agricultural uses of plant biostimulants.” Plant and Soil 383(1-2): 3-41.
Canada, S. (2003). Livestock feed requirements study Canada and provinces 1999, 2000 and 2001. S. Canada. Ottawa, ON, Canada.
Cankar K, Kraigher H, Ravnikar M, Rupnik M (2005) Bacterial endophytes from seeds of Norway spruce (Picea abies L. Karst). FEMS microbiology letters 244: 341-345.
Carrow R (1996) Drought avoidance characteristics of diverse tall fescue cultivars. Crop Sci 36: 371-377.
Charlton, A., J. Donarski, et al. (2008). “Responses of the pea (Pisum sativum L.) leaf metabolome to drought stress assessed by nuclear magnetic resonance spectroscopy.” Metabolomics 4(4): 312-327.
Chaves M M, Oliveira M M (2004) Mechanisms underlying plant resilience to water deficits: prospects for water-saving agriculture. Journal of Experimental Botany 55: 2365-2384.
Chaves, M. M. (1991). “Effects of water deficits on carbon assimilation.” Journal of Experimental Botany 42(1): 1-16.
Chaves, M. M., J. P. Maroco, et al. (2003). “Understanding plant responses to drought—from genes to the whole plant.” Functional plant biology 30(3): 239-264.
Chen, H. and J.-G. Jiang (2010). “Osmotic adjustment and plant adaptation to environmental changes related to drought and salinity.” Environmental Reviews 18(NA): 309-319.
Choudhary D K, Johri B N (2009) Interactions of Bacillus spp. and plants—with special reference to induced systemic resistance (ISR). Microbiological Research 164: 493-513.
Colton-Gagnon K, Ali-Benali M A, Mayer B F, Dionne R, Bertrand A, et al. (2014) Comparative analysis of the cold acclimation and freezing tolerance capacities of seven diploid Brachypodium distachyon accessions. Annals of botany 113: 681-693.
Compant S, Clément C, Sessitsch A (2010) Plant growth-promoting bacteria in the rhizo- and endosphere of plants: their role, colonization, mechanisms involved and prospects for utilization. Soil Biology and Biochemistry 42: 669-678.
Compant S, Duffy B, Nowak J, Clement C, Barka E A (2005) Use of plant growth-promoting bacteria for biocontrol of plant diseases: principles, mechanisms of action, and future prospects. Applied and Environmental Microbiology 71: 4951-4959.
Compant, S., B. Duffy, et al. (2005). “Use of plant growth-promoting bacteria for biocontrol of plant diseases: Principles, mechanisms of action, and future prospects.” Applied and Environmental Microbiology 71(9): 4951-4959.
Couée, I., C. Sulmon, et al. (2006). “Involvement of soluble sugars in reactive oxygen species balance and responses to oxidative stress in plants.” Journal of experimental botany 57(3): 449-459.
Creus, C. M., R. J. Sueldo, et al. (2004). “Water relations and yield in Azospirillum-inoculated wheat exposed to drought in the field.” Canadian Journal of Botany 82(2): 273-281.
Da K, Nowak J, Flinn B (2012) Potato cytosine methylation and gene expression changes induced by a beneficial bacterial endophyte, Burkholderia phytofirmans strain PsJN. Plant Physiology and Biochemistry 50: 24-34.
De Souza, J. and J. V. Da Silva (1987). “Partitioning of carbohydrates in annual and perennial cotton (Gossypium hirsutum L.).” Journal of experimental botany 38(7): 1211-1218.
Dimkpa, C., T. Weinand, et al. (2009). “Plant-rhizobacteria interactions alleviate abiotic stress conditions.” Plant, Cell & Environment 32(12): 1682-1694.
Dordas C (2009) Dry matter, nitrogen and phosphorus accumulation, partitioning and remobilization as affected by N and P fertilization and source-sink relations. European Journal of agronomy 30: 129-139.
Draper J, Mur L A, Jenkins G, Ghosh-Biswas G C, Bablak P, et al. (2001) Brachypodium distachyon. A new model system for functional genomics in grasses. Plant Physiol 127: 1539-1555.
El-Afry, M. M., M. F. El-Nady, et al. (2012). “Anatomical studies on drought-stressed wheat plants (Triticum aestivum L.) treated with some bacterial strains.” Acta Biol. Szeg 56: 165-174.
Elbeltagy A, Nishioka K, Sato T, Suzuki H, Ye B, et al. (2001) Endophytic colonization and in planta nitrogen fixation by a Herbaspirillum sp isolated from wild rice species. Applied and Environmental Microbiology 67: 5285-5293.
Farag, M. A., C.-M. Ryu, et al. (2006). “GC-MS SPME profiling of rhizobacterial volatiles reveals prospective inducers of growth promotion and induced systemic resistance in plants.” Phytochemistry 67(20): 2262-2268.
Farooq, M., A. Wahid, et al. (2009). Plant drought stress: effects, mechanisms and management. Sustainable Agriculture, Springer: 153-188.
Fernandez, O., A. Theocharis, et al. (2012). “Burkholderia phytofirmans PsJN acclimates grapevine to cold by modulating carbohydrate metabolism.” Molecular Plant-Microbe Interactions 25(4): 496-504.
Ferreira A, Quecine M C, Lacava P T, Oda S, Azevedo J L, et al. (2008) Diversity of endophytic bacteria from Eucalyptus species seeds and colonization of seedlings by Pantoea agglomerans. FEMS microbiology letters 287: 8-14.
Figueiredo, M. V., H. A. Burity, et al. (2008). “Alleviation of drought stress in the common bean (Phaseolus vulgaris L.) by co-inoculation with Paenibacillus polymyxa and Rhizobium tropici.” Applied soil ecology 40(1): 182-188.
Gagne-Bourgue F, Aliferis K A, Seguin P, Rani M, Samson R, et al. (2013) Isolation and characterization of indigenous endophytic bacteria associated with leaves of switchgrass (Panicum virgatum L.) cultivars. Journal of Applied Microbiology 114: 836-853.
Gagné-Bourque, F., B. F. Mayer, et al. (2015). “Accelerated growth rate and increased drought stress resilience of the model grass Brachypodium distachyon colonized by Bacillus subtilis B26.” PLoS ONE 10(6): e0130456.
Gill, S. S. and N. Tuteja (2010). “Reactive oxygen species and antioxidant machinery in abiotic stress tolerance in crop plants.” Plant Physiology and Biochemistry 48(12): 909-930.
Glick, B. R. (2012). Plant Growth-Promoting Bacteria: Mechanisms and Applications.
Glick, B. R. (2014). “Bacteria with ACC deaminase can promote plant growth and help to feed the world.” Microbiological Research 169(1): 30-39.
Gontia-Mishra I, Sasidharan S, Tiwari S (2014) Recent developments in use of 1-aminocyclopropane-1-carboxylate (ACC) deaminase for conferring tolerance to biotic and abiotic stress. Biotechnology Letters 36: 889-898.
Gregory P (2006) Roots, rhizosphere and soil: the route to a better understanding of soil science? European Journal of Soil Science 57: 2-12.
H., H. M. and C. J. H. (2008). Agronoy facts 24 Timothy, Penn State College of Agricultural Sciences research and extension programs: 4.
Hallmann J, Berg G (2006) Spectrum and population dynamics of bacterial root endophytes. In: Schulz B E, Boyle C C, Sieber T, editors. Microbial Root Endophytes: Springer Berlin Heidelberg. pp. 15-31.
Hanson, J. and S. Smeekens (2009). “Sugar perception and signaling—an update.” Current Opinion in Plant Biology 12(5): 562-567.
Hardoim, P. R., L. S. van Overbeek, et al. (2008). “Properties of bacterial endophytes and their proposed role in plant growth.” Trends in Microbiology 16(10): 463-471.
Hare, P., W. Cress, et al. (1998). “Dissecting the roles of osmolyte accumulation during stress.” Plant, Cell & Environment 21(6): 535-553.
Harrigan, G. G., L. G. Stork, et al. (2007). “Metabolite analyses of grain from maize hybrids grown in the United States under drought and watered conditions during the 2002 field season.” Journal of agricultural and food chemistry 55(15): 6169-6176.
Hennig A., Kleinschmit J. R. G., Schoneberg S., Löffler S., Janβen A., Polle A. (2015). “Water consumption and biomass production of protoplast fusion lines of poplar hybrids under drought stress” Frontiers in Plant Science 6(330): 1-14.
Hong S Y, Park J H, Cho S H, Yang M S, Park C M (2011) Phenological growth stages of Brachypodium distachyon: codification and description. Weed Research 51: 612-620.
Hong S-Y, Seo P J, Yang M-S, Xiang F, Park C-M (2008) Exploring valid reference genes for gene expression studies in Brachypodium distachyon by real-time PCR. BMC plant biology 8: 112.
Hu, H. Q., X. S. Li, et al. (2010). “Characterization of an antimicrobial material from a newly isolated Bacillus amyloliquefaciens from mangrove for biocontrol of Capsicum bacterial wilt.” Biological Control 54(3): 359-365.
Huang B, Lv C, Zhuang P, Zhang H, Fan L (2011) Endophytic colonisation of Bacillus subtilis in the roots of Robinia pseudoacacia L. Plant Biology 13: 925-931.
Initiative T I B (2010) Genome sequencing and analysis of the model grass Brachypodium distachyon. Nature 463: 763-768.
Jakab, G., J. Ton, et al. (2005). “Enhancing Arabidopsis salt and drought stress tolerance by chemical priming for its abscisic acid responses.” Plant Physiology 139(1): 267-274.
James E K, Gyaneshwar P, Mathan N, Barraquio W L, Reddy P M, et al. (2002) Infection and colonization of rice seedlings by the plant growth-promoting bacterium Herbaspirillum seropedicae Z67. Molecular Plant-Microbe Interactions 15: 894-906.
Jarvis, P. G. (1976). “The interpretation of the variations in leaf water potential and stomatal conductance found in canopies in the field.” Philosophical Transactions of the Royal Society of London B: Biological Sciences 273(927): 593-610.
Ji, X., G. Lu, et al. (2008). “Biological control against bacterial wilt and colonization of mulberry by an endophytic Bacillus subtilis strain.” Fems Microbiology Ecology 65(3): 565-573.
Kasim, W. A., M. E. Osman, et al. (2013). “Control of drought stress in wheat using plant-growth-promoting bacteria.” Journal of plant growth regulation 32(1): 122-130.
Kempf, B. and E. Bremer (1998). “Uptake and synthesis of compatible solutes as microbial stress responses to high-osmolality environments.” Archives of Microbiology 170(5): 319-330.
Khan, N. H., D. R. Korber, N. H. Low and M. T. Nickerson (2013). “Development of extrusion-based legume protein isolate-alginate capsules for the protection and delivery of the acid sensitive probiotic, Bifidobacterium adolescentis.” Food Research International 54(1): 730-737.
Kinnersley, A. M. and F. J. Turano (2000). “Gamma aminobutyric acid (GABA) and plant responses to stress.” Critical Reviews in Plant Sciences 19(6): 479-509.
Krasensky, J. and C. Jonak (2012). “Drought, salt, and temperature stress-induced metabolic rearrangements and regulatory networks.” Journal of Experimental Botany.
Kusaka, M., M. Ohta, et al. (2005). “Contribution of inorganic components to osmotic adjustment and leaf folding for drought tolerance in pearl millet.” Physiologia Plantarum 125(4): 474-489.
Lea, P. J., L. Sodek, et al. (2007). “Asparagine in plants.” Annals of Applied Biology 150(1): 1-26.
Leme\check{z}ienė, N., J. Kanapeckas, et al. (2004). “Analysis of dry matter yield structure of forage grasses.” Plant Soil Environment 50(6): 277-282.
Leport, L., N. Turner, et al. (1999). “Physiological responses of chickpea genotypes to terminal drought in a Mediterranean-type environment.” European Journal of Agronomy 11(3): 279-291.
Less, H. and G. Galili (2008). “Principal transcriptional programs regulating plant amino acid metabolism in response to abiotic stresses.” Plant Physiology 147(1): 316-330.
Liu Q, Kasuga M, Sakuma Y, Abe H, Miura S, et al. (1998) Two transcription factors, DREB1 and DREB2, with an EREBP/AP2 DNA binding domain separate two cellular signal transduction pathways in drought- and low-temperature-responsive gene expression, respectively, in arabidopsis. The Plant Cell Online 10: 1391-1406.
Liu, B., H. Qiao, et al. (2009). “Biological control of take-all in wheat by endophytic Bacillus subtilis El R-j and potential mode of action.” Biological Control 49(3): 277-285.
Livak K J, Schmittgen T D (2001) Analysis of relative gene expression data using real-time quantitative PCR and the 2-ΔΔCT method. methods 25: 402-408.
Livingston III D P, Hincha D K, Heyer A G (2009) Fructan and its relationship to abiotic stress tolerance in plants. Cellular and Molecular Life Sciences 66: 2007-2023.
Lucy, M., E. Reed, et al. (2004). “Applications of free living plant growth-promoting rhizobacteria.” Antonie van Leeuwenhoek 86(1): 1-25.
Lugtenberg, B. and F. Kamilova (2009). “Plant-growth-promoting rhizobacteria.” Annual review of microbiology 63: 541-556.
Madkour, M. A., L. T. Smith, et al. (1990). “Preferential osmolyte accumulation: a mechanism of osmotic stress adaptation in diazotrophic bacteria.” Applied and environmental microbiology 56(9): 2876-2881.
Marulanda, A., J.-M. Barea, et al. (2009). “Stimulation of plant growth and drought tolerance by native microorganisms (A M fungi and bacteria) from dry environments: mechanisms related to bacterial effectiveness.” Journal of Plant Growth Regulation 28(2): 115-124.
Marulanda, A., R. Azcón, et al. (2010). “Regulation of plasma membrane aquaporins by inoculation with a strain in maize (L.) plants under unstressed and salt-stressed conditions.” Planta 2(232): 533-543.
Mei, C. and B. S. Flinn (2010). “The use of beneficial microbial endophytes for plant biomass and stress tolerance improvement.” Recent Patents on Biotechnology 4(1): 81-95.
Meister, R., M. S. Rajani, et al. (2014). “Challenges of modifying root traits in crops for agriculture.” Trends in Plant Science 19(12): 779-788.
Mena-Violante H G, Olalde-Portugal V (2007) Alteration of tomato fruit quality by root inoculation with plant growth-promoting rhizobacteria (PGPR): Bacillus subtilis BEB-13bs. Scientia Horticulturae 113: 103-106.
Miazek, A., J. Bogdan, et al. (2001). “Effects of water deficit during germination of wheat seeds.” Biologia Plantarum 44(3): 397-403.
Muurinen S, Kleemola J, Peltonen-Sainio P (2007) Accumulation and translocation of nitrogen in spring cereal cultivars differing in nitrogen use efficiency. Agronomy Journal 99: 441-449.
Nadeem S M, Ahmad M, Zahir Z A, Javaid A, Ashraf M (2014) The role of mycorrhizae and plant growth promoting rhizobacteria (PGPR) in improving crop productivity under stressful environments. Biotechnology Advances 32: 429-448.
Nagabhyru, P., R. D. Dinkins, et al. (2013). “Tall fescue endophyte effects on tolerance to water-deficit stress.” BMC plant biology 13(1): 127.
Nautiyal, C. S., S. Srivastava, et al. (2013). “Plant growth-promoting bacteria Bacillus amyloliquefaciens NBRISN13 modulates gene expression profile of leaf and rhizosphere community in rice during salt stress.” Plant Physiology and Biochemistry 66(0): 1-9.
Naveed, M., B. Mitter, et al. (2014). “Increased drought stress resilience of maize through endophytic colonization by Burkholderia phytofirmans PsJN and Enterobacter sp. FD17.” Environmental and Experimental Botany 97: 30-39.
Niu, D.-D., H.-X. Liu, et al. (2011). “The plant growth-promoting rhizobacterium Bacillus cereus AR156 induces systemic resistance in Arabidopsis thaliana by simultaneously activating salicylate- and jasmonate/ethylene-dependent signaling pathways.” Molecular Plant-Microbe Interactions 24(5): 533-542.
Ongena M, Jacques P (2008) Bacillus lipopeptides: versatile weapons for plant disease biocontrol. Trends in Microbiology 16: 115-125.
Orhan E, Esitken A, Ercisli S, Turan M, Sahin F (2006) Effects of plant growth promoting rhizobacteria (PGPR) on yield, growth and nutrient contents in organically growing raspberry. Scientia Horticulturae 111: 38-43.
Ortiz-Castro, R., E. Valencia-Cantero, et al. (2008). “Plant growth promotion by Bacillus megaterium involves cytokinin signaling.” Plant signaling & behavior 3(4): 263-265.
Parker D, Beckmann M, Enot D P, Overy D P, Rios Z C, et al. (2008) Rice blast infection of Brachypodium distachyon as a model system to study dynamic host/pathogen interactions. Nat Protocols 3: 435-445.
Parkinson J, Allen S (1975) A wet oxidation procedure suitable for the determination of nitrogen and mineral nutrients in biological material. Communications in Soil Science & Plant Analysis 6: 1-11.
Paul, D. and S. Nair (2008). “Stress adaptations in a plant growth promoting rhizobacterium (PGPR) with increasing salinity in the coastal agricultural soils.” Journal of basic microbiology 48(5): 378.
Penrose, D. M. and B. R. Glick (2003). “Methods for isolating and characterizing ACC deaminase-containing plant growth-promoting rhizobacteria.” Physiologia Plantarum 118(1): 10-15.
Pérez-Garcia A, Romero D, De Vicente A (2011) Plant protection and growth stimulation by microorganisms: biotechnological applications of Bacilli in agriculture. Current Opinion in Biotechnology 22: 187-193.
Porebski, S., L. G. Bailey, et al. (1997). “Modification of a CTAB DNA extraction protocol for plants containing high polysaccharide and polyphenol components.” Plant Molecular Biology Reporter 15(1): 8-15.
Poupin, M. J., T. Timmermann, et al. (2013). “Effects of the plant growth-promoting bacterium Burkholderia phytofirmans PsJN throughout the life cycle of Arabidopsis thaliana.” Plos One 8(7).
Québec, V. g. d. (2008). Rapport du Vérificateur général du Québec à l'Assemblée Nationale pour l'année 2007-2008. Québec, Institue du Vérificateur général du Québec. Tome 2: Ch. 1, p. 8.
Rai, R., P. Dash, et al. (2007). “Endophytic bacterial flora in the stem tissue of a tropical maize (Zea mays L.) genotype: isolation, identification and enumeration.” World Journal of Microbiology and Biotechnology 23(6): 853-858.
Ran, F. A., P. D. Hsu, et al. (2013). “Genome engineering using the CRISPR-Cas9 system.” Nat. Protocols 8(11): 2281-2308.
Reddy, A. R., K. V. Chaitanya, et al. (2004). “Drought-induced responses of photosynthesis and antioxidant metabolism in higher plants.” Journal of plant physiology 161(11): 1189-1202.
Reinhold-Hurek B, Hurek T (2011) Living inside plants: bacterial endophytes. Current Opinion in Plant Biology 14: 435-443.
Reva, O. N., C. Dixelius, et al. (2004). “Taxonomic characterization and plant colonizing abilities of some bacteria related to Bacillus amyloliquefaciens and Bacillus subtilis.” FEMS Microbiology Ecology 48(2): 249-259.
Richardson, M. D., G. W. Chapman, et al. (1992). “Sugar alcohols in endophyte-infected tall fescue under drought.” Crop Sci. 32(4): 1060-1061.
Rocha F R, Papini-Terzi F S, Nishiyama M Y, Vêncio R Z, Vicentini R, et al. (2007) Signal transduction-related responses to phytohormones and environmental challenges in sugarcane. BMC genomics 8: 71.
Rodríguez, H. and R. Fraga (1999). “Phosphate solubilizing bacteria and their role in plant growth promotion.” Biotechnology advances 17(4): 319-339.
Rosenblueth M, Martinez-Romero E (2006) Bacterial endophytes and their interactions with hosts. Molecular Plant-Microbe Interactions 19: 827-837.
Ruan, Y.-L., Y. Jin, et al. (2010). “Sugar input, metabolism, and signaling mediated by invertase: Roles in development, yield potential, and response to drought and heat.” Molecular Plant 3(6): 942-955.
Ryu, C.-M., M. A. Farag, et al. (2003). “Bacterial volatiles promote growth in Arabidopsis.” Proceedings of the National Academy of Sciences 100(8): 4927-4932.
Sakuma Y, Maruyama K, Osakabe Y, Qin F, Seki M, et al. (2006) Functional analysis of an Arabidopsis transcription factor, DREB2A, involved in drought-responsive gene expression. The Plant Cell Online 18: 1292-1309.
Saleem, M., M. Arshad, et al. (2007). “Perspective of plant growth promoting rhizobacteria (PGPR) containing ACC deaminase in stress agriculture.” Journal of Industrial Microbiology & Biotechnology 34(10): 635-648.
Sandhya, V., S. Z. Ali, et al. (2010). “Effect of plant growth promoting Pseudomonas spp. on compatible solutes, antioxidant status and plant growth of maize under drought stress.” Plant Growth Regulation 62(1): 21-30.
Sarma R K, Saikia R (2014) Alleviation of drought stress in mung bean by strain Pseudomonas aeruginosa GGRJ21. Plant and soil 377: 111-126.
Sharma, S. S. and K.-J. Dietz (2006). “The significance of amino acids and amino acid-derived molecules in plant responses and adaptation to heavy metal stress.” Journal of Experimental Botany 57(4): 711-726.
Simon, U. and B. Park (1983). “A descriptive scheme for stages of development in perennial forage grasses.”
Skinner, F., P. Jones, et al. (1952). “A comparison of a direct- and a plate-counting technique for the quantitative estimation of soil micro-organisms.” Journal of General Microbiology 6(3-4): 261-271.
Spollen, W. G. and C. J. Nelson (1994). “Response of fructan to water deficit in growing leaves of tall fescue.” Plant Physiology 106(1): 329-336.
Sun, L., Z. Lu, et al. (2006). “Isolation and characterization of a co-producer of fengycins and surfactins, endophytic Bacillus amyloliquefaciens ES-2, from Scutellaria baicalensis Georgi.” World Journal of Microbiology and Biotechnology 22(12): 1259-1266.
Sziderics, A., F. Rasche, et al. (2007). “Bacterial endophytes contribute to abiotic stress adaptation in pepper plants (Capsicum annuum L.).” Canadian journal of microbiology 53(11): 1195-1202.
Taji, T., C. Ohsumi, et al. (2002). “Important roles of drought- and cold-inducible genes for galactinol synthase in stress tolerance in Arabidopsis thaliana.” The Plant Journal 29(4): 417-426.
Timmusk, S. and E. G. H. Wagner (1999). “The plant-growth-promoting rhizobacterium Paenibacillus polymyxa induces changes in Arabidopsis thaliana gene expression: A possible connection between biotic and abiotic stress responses.” Molecular Plant-Microbe Interactions 12(11): 951-959.
Truyens S, Weyens N, Cuypers A, Vangronsveld J (2014) Bacterial seed endophytes: genera, vertical transmission and interaction with plants. Environmental Microbiology Reports.
Truyens, S., N. Weyens, et al. (2014). “Bacterial seed endophytes: genera, vertical transmission and interaction with plants.” Environmental Microbiology Reports 7(1): 10.
Vain, P., B. Worland, V. Thole, N. McKenzie, S. C. Alves, M. Opanowicz, L. J. Fish, M. W. Bevan and J. W. Snape (2008). “Agrobacterium-mediated transformation of the temperate grass Brachypodium distachyon (genotype Bd21) for T-DNA insertional mutagenesis.” Plant Biotechnology Journal 6(3): 236-245.
Valliyodan B, Nguyen H T (2006) Understanding regulatory networks and engineering for enhanced drought tolerance in plants. Current Opinion in Plant Biology 9: 189-195.
van Elsas, J. D., A. F. Dijkstra, et al. (1986). “Survival of Pseudomonas fluorescens and Bacillus subtilis introduced into two soils of different texture in field microplots.” FEMS Microbiology Letters 38(3): 151-160.
Van Loon, L. (2007). “Plant responses to plant growth-promoting rhizobacteria.” European Journal of Plant Pathology 119(3): 243-254.
Vardharajula, S., S. Zulfikar Ali, et al. (2011). “Drought-tolerant plant growth promoting Bacillus spp.: effect on growth, osmolytes, and antioxidant status of maize under drought stress.” Journal of Plant Interactions 6(1): 1-14.
Venekamp, J. (1989). “Regulation of cytosol acidity in plants under conditions of drought.” Physiologia Plantarum 76(1): 112-117.
Verslues, P. E. and S. Sharma (2010). “Proline metabolism and its implications for plant-environment interaction.” The Arabidopsis Book 8: e0140.
Vivas, A., A. Marulanda, et al. (2003). “Influence of a Bacillus sp. on physiological activities of two arbuscular mycorrhizal fungi and on plant responses to PEG-induced drought stress.” Mycorrhiza 13(5): 249-256.
Vogel J P, Garvin D F, Mockler T C, Schmutz J, Rokhsar D, et al. (2010) Genome sequencing and analysis of the model grass Brachypodium distachyon. Nature 463: 763-768.
Wahyudi, A. T., R. P. Astuti, et al. (2011). “Characterization of Bacillus spp. strains isolated from rhizosphere of soybean plants for their use as potential plant growth for promoting Rhizobacteria.” Journal of Microbiology and Antimicrobials 3(2): 6.
Wang W-S, Pan Y-J, Zhao X-Q, Dwivedi D, Zhu L-H, et al. (2010) Drought-induced site-specific DNA methylation and its association with drought tolerance in rice (Oryza sativa L.). Journal of Experimental Botany.
Wani, P. A. and M. S. Khan (2010). “Bacillus species enhance growth parameters of chickpea (Cicer arietinum L.) in chromium stressed soils.” Food and chemical toxicology 48(11): 3262-3267.
Whatmore, A. M., J. A. Chudek, et al. (1990). “The effects of osmotic upshock on the intracellular solute pools of Bacillus subtilis.” Journal of General Microbiology 136(12): 2527-2535.
Witt, S., L. Galicia, et al. (2011). “Metabolic and phenotypic responses of greenhouse-grown maize hybrids to experimentally controlled drought stress.” Molecular plant: ssr102.
Wolter A, Schroeder F-G. Effect of Drought Stress on the Productivity of Ivy Treated with Rhizobacterium Bacillus subtilis; 2012. pp. 107-113.
Wolter, A. and F.-G. Schroeder (2012). Effect of drought stress on the productivity of ivy treated with rhizobacterium Bacillus subtilis. International Symposium on Soilless Cultivation 1004.
Woodman, M. E. (2008). “Direct PCR of intact bacteria (colony PCR).” Current protocols in microbiology: A. 3D. 1-A. 3D. 6.
Wulff E G, van Vuurde J W L, Hockenhull J (2003) The ability of the biological control agent Bacillus subtilis, strain BB, to colonise vegetable brassicas endophytically following seed inoculation. Plant and Soil 255: 463-474.
Xu, M., J. Sheng, et al. (2014). “Bacterial community compositions of tomato (Lycopersicum esculentum Mill.) seeds and plant growth promoting activity of ACC deaminase producing Bacillus subtilis (HYT-12-1) on tomato seedlings.” World Journal of Microbiology and Biotechnology 30(3): 835-845.
Yang, J., J. W. Kloepper, et al. (2009). “Rhizosphere bacteria help plants tolerate abiotic stress.” Trends in Plant Science 14(1): 1-4.
Yasbin, R. E., G. A. Wilson and F. E. Young (1975). “Transformation and transfection in lysogenic strains of Bacillus subtilis: evidence for selective induction of prophage in competent cells.” Journal of Bacteriology 121(1): 296-304.
Zahir, Z., A. Munir, et al. (2008). “Effectiveness of rhizobacteria containing ACC deaminase for growth promotion of peas (Pisum sativum) under drought conditions.” J Microbiol Biotechnol 18(5): 958-963.
Zhang, S. and W. Outlaw (2001). “Abscisic acid introduced into the transpiration stream accumulates in the guard-cell apoplast and causes stomatal closure.” Plant, Cell & Environment 24(10): 1045-1054.

1. A method of increasing drought resistance of a plant, the method comprising applying a Bacillus methylotrophicus or a composition thereof (i) to the plant or to a part of the plant; and/or (ii) to an area around the plant or plant part, in an amount effective to produce an increased drought resistance in the plant as compared to the drought stress resistance of the plant in the absence of said application of Bacillus methylotrophicus or composition.

2. The method of claim 1, wherein the Bacillus methylotrophicus exhibits one or more of (1) an ability to form sustaining endophytic populations in all tissues of the plant as well as in the rhizosphere; (2) an ability to avoid triggering the plant immune system; (3) an ability to reduce signs of wilting in the plant or increase survival time of the plant in drought conditions; (4) an ability to increase expression of at least one drought-responsive genes in the plant; (5) an ability to increase starch in the plant; (6) an ability to increase total soluble sugars in the plant; (7) an ability to increase DNA methylation in bacterized plant; (8) an ability to increase expression of at least one DNA methyltransferase in the plant; (9) an ability to maintain or increase crop biomass of the plant; (10) an ability to maintain or increase photosynthesis of the plant; (11) an ability to maintain or increase water conductance of the plant; (12) an ability to increase total amino acids content in roots and/or in shoots of the plant; (13) an ability to increase amino asparagine, glutamic acid and/or glutamine content in roots and/or in shoots of the plant; and (14) an ability to increase non-protein amino acid GABA in shoots and/or roots of the plant.

3. The method of claim 1, wherein the Bacillus methylotrophicus exhibits one or more of (3) an ability to reduce signs of wilting in the plant or increase survival time of the plant in drought conditions; (4) an ability to increase expression of at least one drought-responsive genes in the plant; (5) an ability to increase starch in the plant; (6) an ability to increase total soluble sugars in the plant; (7) an ability to increase DNA methylation in bacterized plant; (8) an ability to increase expression of at least one DNA methyltransferase in the plant; (9) an ability to maintain or increase crop biomass of the plant; (10) an ability to maintain or increase photosynthesis of the plant; (11) an ability to maintain or increase water conductance of the plant; (12) an ability to increase total amino acids content in roots and/or in shoots of the plant; (13) an ability to increase amino asparagine, glutamic acid and/or glutamine content in roots and/or in shoots of the plant; and (14) an ability to increase non-protein amino acid GABA in shoots and/or roots of the plant, under drought conditions.

4. The method of claim 1, wherein the Bacillus methylotrophicus exhibits one or more of the characteristics (23) to (31) defined in Table 1.

5. The method of claim 1, wherein the Bacillus methylotrophicus is 1-aminocyclopropane-1-carboxylate (ACC) deaminase deficient.

6. The method of claim 1, wherein the plant is a poaceae plant, preferably a food crop plant.

7. (canceled)

8. The method of claim 1, wherein (a) the amount effective is about 1×10⁸ CFU or more/plant, plant part, or area around a plant or plant part; and/or (b) the Bacillus methylotrophicus is in a seed of a second generation plant infected with the Bacillus methylotrophicus.

9. (canceled)

10. The method of claim 1, wherein the composition of Bacillus methylotrophicus comprises a polymer wherein said polymer is mixed and extruded with said Bacillus methylotrophicus in a proportion of 10 to 1, and preferably the polymer is pea protein and/or alginate.

11. (canceled)

12. The method of claim 1, wherein the Bacillus methylotrophicus is of a strain comprising all of the biochemical characteristics of a Bacillus methylotrophicus deposited at the ATCC under accession no. PTA-122326 on Jul. 21, 2015, or a mutant thereof isolated from said strain and able to induce drought resistance to the plant.

13. A biologically pure culture of a 1-aminocyclopropane-1-carboxylate (ACC) deaminase deficient Bacillus methylotrophicus bacterium strain, or a mutant thereof able to induce drought resistance in a plant.

14. The Bacillus methylotrophicus bacterium strain, or mutant thereof of claim 13, wherein the strain or mutant thereof exhibits one or more of (1) an ability to form sustaining endophytic populations in all tissues of the plant as well as in the rhizosphere; (2) an ability to avoid triggering the plant immune system; (3) an ability to reduce signs of wilting in the plant or increase survival time of the plant in drought conditions; (4) an ability to increase expression of at least one drought-responsive genes in the plant; (5) an ability to increase starch in the plant; (6) an ability to increase total soluble sugars in the plant; (7) an ability to increase DNA methylation in bacterized plant; (8) an ability to increase expression of at least one DNA methyltransferase in the plant; (9) an ability to maintain or increase crop biomass of the plant; (10) an ability to maintain or increase photosynthesis of the plant; (11) an ability to maintain or increase water conductance of the plant; (12) an ability to increase total amino acids content in roots and/or in shoots of the plant; (13) an ability to increase amino asparagine, glutamic acid and/or glutamine content in roots and/or in shoots of the plant; and (14) an ability to increase non-protein amino acid GABA in shoots and/or roots of the plant.

15. The Bacillus methylotrophicus bacterium strain, or mutant thereof of claim 13, wherein the strain or mutant exhibits one or more of (3) an ability to reduce signs of wilting in the plant or increase survival time of the plant in drought conditions; (4) an ability to increase expression of at least one drought-responsive genes in the plant; (5) an ability to increase starch in the plant; (6) an ability to increase total soluble sugars in the plant; (7) an ability to increase DNA methylation in bacterized plant; (8) an ability to increase expression of at least one DNA methyltransferase in the plant; (9) an ability to maintain or increase crop biomass of the plant; (10) an ability to maintain or increase photosynthesis of the plant; (11) an ability to maintain or increase water conductance of the plant; (12) an ability to increase total amino acids content in roots and/or in shoots of the plant; (13) an ability to increase amino asparagine, glutamic acid and/or glutamine content in roots and/or in shoots of the plant; and (14) an ability to increase non-protein amino acid GABA in shoots and/or roots of the plant, under drought conditions.

16. The Bacillus methylotrophicus bacterium strain, or mutant thereof of claim 13, wherein the strain or mutant exhibits one or more of the characteristics (23) to (31) defined in Table 1.

17. A biologically pure culture of a bacterium strain comprising all of the biochemical characteristics of a Bacillus methylotrophicus deposited at the ATCC under accession no. PTA-122326 on Jul. 21, 2015, or a mutant thereof isolated from said strain and able to induce drought resistance to a plant.

18. A composition comprising the bacterium strain or mutant thereof defined in claim 13, and at least one carrier, preferably wherein the carrier comprises a polymer wherein said polymer is mixed and extruded with said bacterium strain or mutant thereof in a proportion of about 10 to about 1, and more preferably the polymer is pea protein and/or alginate.

19. (canceled)

20. (canceled)

21. A seed coated with the bacterium strain or mutant thereof defined in claim 13, or with a composition comprising the bacterium strain or mutant thereof, and at least one carrier.

22. A second or subsequent generation seed of a plant infected with a bacterium strain or with a mutant thereof, the bacterium strain or a mutant thereof being as defined claim 13.

23. A method of increasing a plant's growth, the method comprising applying the bacterium strain or mutant thereof defined in claim 13, or a composition comprising the bacterium strain or mutant thereof, and at least one carrier, (i) to the plant or to a part of the plant; and/or (ii) to an area around the plant or plant part in an amount effective to produce an increased plant growth as compared to the growth of the plant in the absence of said application of Bacillus methylotrophicus or composition.

24. The method of claim 23, wherein the plant is a poaceae plant, preferably a food crop plant.

25. (canceled)

26. The method of claim 23, wherein (a) the amount effective is about 1×10⁸ CFU or more/plant, plant part, or area around a plant or plant part; and/or (b) the bacterium strain or mutant thereof is in a seed of a second generation plant infected with the bacterium strain or mutant thereof.

27. (canceled)
